SEQUENCE LISTING (4 sequences)

SEQ ID NO: 1 - protein, 31 aa, synthetic construct
  REGION 1..31: note = Fc peptide fragment
TCPPCPAPELLGGPSVFLFPPKPKDTLMISR

SEQ ID NO: 2 - protein, 33 aa, synthetic construct
  REGION 1..33: note = IgG peptide fragment
THTCPPCPAPELLGGPSVFLFPPKPKDTLMISR

SEQ ID NO: 3 - protein, 25 aa, synthetic construct
  REGION 1..25: note = Fc peptide fragment
LLGGPSVFLFPPKPKDTLMISRTPE

SEQ ID NO: 4 - protein, 13 aa, Homo sapiens
DCAWHLGELVWCT

(19) United States
(12) Patent Application Publication
(10) Pub. No.: US 2024/0024504 A1
(43) Pub. Date: Jan. 25, 2024

(54) SITE-SELECTIVE LYSINE ACETYLATION OF HUMAN IMMUNOGLOBULIN G AND IgG-RELATED PRODUCTS FOR IMMUNOTHERAPY

(71) Applicant: The Chinese University of Hong Kong, Hong Kong (CN)

(72) Inventors: Jiang XIA, Hong Kong (CN); Yu ZHANG, Anhui (CN); Dingdong YUAN, Hangzhou (CN)

(21) Appl. No.: 18/048,547

(22) Filed: Oct. 21, 2022

Related U.S. Application Data
(60) Provisional application No. 63/368,573, filed on Jul. 15, 2022.

(51) Int. Cl. A61K 47/69; C07K 7/56; A61K 47/60; A61K 47/68

(52) U.S. Cl. CPC A61K 47/6913; C07K 7/56; A61K 47/60; A61K 47/6835; A61K 47/6811

(57) ABSTRACT

The subject invention pertains to methods of acetylation of the Fc region of immunoglobulins, particularly Lys248 of the heavy chain of human Immunoglobulin G (IgG) using a novel Fc-III derived peptide. The acetylation reaction with the phenolic ester with an azide or alkyne enables site-selective functionalization of Lys248 with a bioorthogonal reactive group for further derivatization. Further methods are provided to synthesize an antibody-lipid conjugate that allows for the construction of an immunoliposome that can target cells expressing oncogenes, including HER2+ cells, and a bispecific antibody complex (bsAbC) linking two distinct antibodies. The bsAbC can induce an effector-cell mediated cytotoxicity at nanomolar concentrations.

### CROSS-REFERENCE TO RELATED APPLICATION

This application claims the benefit of U.S. Provisional Application Ser. No. 63/368,573, filed Jul. 15, 2022, which is hereby incorporated by reference in its entirety including any tables, figures, or drawings.

### SEQUENCE LISTING

The Sequence Listing for this application is labeled “CUHK.191X-SeqList-as filed.xml” which was created on Oct. 20, 2022 and is 5,658 bytes. The entire contents of the sequence listing is incorporated herein by reference in its entirety.

### BACKGROUND OF THE INVENTION

Immunoglobin G (IgG) antibodies are a class of large Y-shaped proteins generated mainly by the plasma cells used by the immune system for targeting and neutralizing foreign substances, such as viruses and bacteria. Due to their excellent capability of binding with specific molecules (antigens) or parts of them (epitopes), antibodies are widely used in laboratories for chemical and biological uses including immunoprecipitation, protein isolation, Western blotting analysis, immunofluorescence, among many others. Many monoclonal antibodies (mAbs) are also used for immunotherapy; since the approval of muromonab-CD3 by the Food and Drug Administration (FDA) for the treatment of acute rejection in patients with organ transplants in 1986, many antibody drugs have entered clinical use or in clinical trials [1].

Derivatives of antibodies have also been developed to enhance the therapeutic index of therapeutic mAbs. For example, bispecific antibodies (bsAbs) can bind with two different types of antigens or two epitopes of the same antigen and have been successfully used in immunotherapies of cancer. Immunoliposomes, antibody functionalized liposomes, are advanced frontiers of targeted cancer therapies. Antibody-drug conjugates (ADCs) bring cytotoxic anticancer drugs to tumor cells while sparing healthy cells [2, 3]. Altogether, all these applications are built on new approaches to modify and/or engineer antibodies, in particular IgGs. Among these new approaches, the development of bioconjugation and biorthogonal chemistry of IgGs is of paramount importance [4-7].

Chemical functionalization of antibodies entered a booming phase since the approval of the first antibody-drug conjugates (ADCs) by the US FDA for targeted cancer therapies in 2000 [1]. Notwithstanding a bright potential, only a limited number of synthetic methods have been developed for antibody functionalization to date [1, 2]. One of the key challenges is the precise control of the reaction sites; failure to achieve this results in a heterogeneous population of the ADC molecules which complicates clinical use [3, 4]. Chemists have been seeking to activate existing chemical groups on the antibodies through various methods. Breaking the disulfide structure [8-10] or modifying the surface glycans [11] created reactive groups with activity above the basal level for conjugation reactions. Or, proximity-induced (affinity-guide or ligand-directed) reactions can realize site-selective reactions by local confinement; examples include a glycan-directed tosyl reaction [12], light-activated benzoyl-phenylalanine reaction driven by IgG-protein G interaction [13], IgG conjugation reaction through the 4-fluorophenyl carbamate lysine driven by IgG-FB protein interaction [14], IgG-peptide conjugation through a DSG crosslinker [15, 16], and site-specific modification of asparagine-79 by a hexarhodium metallopeptide catalyst driven by IgG-peptide interaction [17]. Notwithstanding these successes, the chemical reactions above still hardly match with the post-translational protein reactions catalyzed by dedicated enzymes in nature: often either a bulky stub was left on the protein, a protein partner with a sophisticated unnatural amino acid incorporated was required, or the reaction failed to achieve single-residue resolution. An acetyltransferase, for example, can install a two-carbon unit (acetylation) at a single lysine residue with unmistakable accuracy [18].

Imitating the mechanism of enzyme-catalyzed reactions, we have been using proximity-induced reactivity to achieve unparalleled substrate fidelity and site accuracy of protein conjugation or modification reactions. [19-25] Cysteine residues exposed on the surface of proteins are excellent targets for protein modifications, but the thioester as the result of the nucleophilic reaction is often not stable. [26] Most of the cysteine reactions also leave a bulky stub on the proteins of interest, which may jeopardize its function. Covalent reactions of lysine residues at the ε-amino group are favorable because of the abundance of lysine in most proteins and the stability of the resultant amide bonds. Lysine reactions are amenable to site-selective control through proximity-induced reactivity and even in ubiquitin-like proteins which are small and rich in lysine residues, proximity-induced lysine reactions can be confined to one or two lysine residues. [21] Lysine acetyltransferases catalyze the transfer of the acetyl group of acetyl-CoA to the ε-amino group of an internal lysine residue in the substrate, often histone proteins. [27, 28]

Therefore, there remains a need for novel means and methods for immunotherapy.

### BRIEF SUMMARY OF THE INVENTION

The subject invention pertains to methods of acetylation of the Fc region of immunoglobulins, particularly Lys248 of the heavy chain of human Immunoglobulin G (IgG) using a novel Fc-III derived peptide. In certain embodiments, the Fc-III derived peptide has a glutamine derivative that contains a phenyl azidoacetate motif at the side chain substituted for at least one amino acid residue of Fc-III.

In certain embodiments, the acetylation reaction with the phenolic ester with an azide or alkyne enables site-selective functionalization of Lys248 with a bioorthogonal reactive group for further derivatization. Further methods are provided to synthesize an antibody-lipid conjugate that allows for the construction of an immunoliposome that can target cells expressing oncogenes, including HER2+ cells, and a bispecific antibody complex (bsAbC) linking two distinct antibodies. The bsAbC can induce an effector-cell mediated cytotoxicity at nanomolar concentrations.

An IgG contains over 80 lysine residues, among which, 20 of them are found at highly solvent-accessible sites. [29] In certain embodiments, a phenolic ester can be used to imitate the activated acetyl group carrier (acetyl-CoA), the Fc domain of the IgG as the substrate, and an Fc-binding peptide to mimic the framework of lysine acetyltransferases, which recognizes and stably binds the substrate. The proximity due to the binding interaction realizes spontaneous acetylation of Lys248 of the Fc domain (without modifying the rest 80-90 lysine residues in IgGs).

Bispecific antibodies (bsAbs) recognize two antigens or two different epitopes on the same antigen. Three bsAbs blinatumomab, emicizumab, and amivantamab are in clinical use and many are promising candidates in clinical trials. The synthesis of bsAbs requires new antibody constructs instead of native IgGs. In certain embodiments, the subject methods pertain to novel antibody functionalization methods that allows for the construction of bispecific antibody complexes (bsAbs) with efficacy in immunotherapy.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-1E Lysine acetylation of IgG Fc. (FIG. 1A) Crystal structure of Fc-III in complex with an Fc region (PDB ID: 1DN2). (FIG. 1B) Design of azidoacetyl peptides F1 to F3. (FIG. 1C) Scheme to show the transfer of azidoacetyl group from peptide to Lys 248 of IgG Fc by proximal reaction and fluorescence labeling based on the strain-promoted azide-alkyne cycloaddition, or SPAAC. (FIG. 1D) Coomassie-stained gel and fluorescent image to show successful acetylation by peptide F1. Lane M, molecular weight marker; lane 1, IgG Fc (5 μg); lane 2, IgG Fe (5 μg) and peptide F1; lane 3, IgG Fe (5 μg) and peptide F2; lane 4, IgG Fc (5 μg) and peptide F3. Reaction condition: 10 μM IgG Fc, 60 μM peptide, PBS buffer (pH 7.4), at 37° C. for 1 h. The reactions were quenched with 1% SDS, and DBCO-PEG4-TAMRA was added at room temperature for 1.5 h. Then the solutions were then resolved by denaturing SDS-PAGE and imaged by in-gel fluorescence scanning and Coomassie blue staining. (FIG. 1E) MALDI-TOF MS analysis of the modified Fc region at a reduced form. M-Fc: modified Fc region.

FIG. 2A-2C Kinetic details of the acetylation reaction and hydrolysis of F1. (FIG. 2A) Reactions at different peptide-to-IgG Fc ratios. Reactions were performed in PBS buffer (pH 7.4) at 37° C. for 1 h. Protein loading in each lane is 2 μg. (FIG. 2B) Reaction kinetics. Reaction condition: 4 μM IgG Fc, 24 μM F1, PBS buffer (pH 7.4), at 37° C. The reactions were quenched with 1% SDS at different time points. After conducting an SPAAC reaction with DBCO-PEG4-TAMRA, the reactions were denatured and resolved by SDS-PAGE. Product conversion was quantified based on band-shift in the SDS-PAGE image. (FIG. 2C) Hydrolysis of peptide ester (24 μM) monitored by HPLC in pH 7.4 PBS buffer at 37° C.

FIG. 3A-3C The effect of the peptide structure on acetylation reaction. (FIG. 3A) The structures of peptides used in this work. FITC moiety was added at the N-termini of the peptides to MST analysis. (FIG. 3B) Competition between reactive peptide (F1) and non-reactive peptide (f-F4 or f-F0). The binding affinity (Kd) of peptide f-F0 (11.7 nM), f-F4 (1.5 μM) and f-F5 (F1 analogue) (2.0 μM) was detected by MST. (FIG. 3C) Crystal structure of Fc-III in complex with Fc region (PDB ID: 1DN2) (left) and the reactivity comparison between F1 and F6 with the linker of 4-aminophenol and 3-aminophenol, respectively (right).

FIG. 4A-4C Acetylation of atezolizumab. (FIG. 4A) Acetylation reaction monitored by SDS-PAGE and fluorescent scanning. Reaction condition: 4 μM Atezo, 24 μM F1, PBS buffer (pH 7.4), at 37° C. for 1 h. The reactions were quenched with 1% SDS and DBCO-TAMRA was added at room temperature for 1.5 h for SPAAC reaction before SDS-PAGE and in-gel fluorescent imaging. Atezolizumab loading in each lane is 6 μg. (FIG. 4B) MALDI-TOF MS analysis of atezolizumab acetylation at different time points. (FIG. 4C) In-gel digestion for MALDI-TOF MS analysis to identify the acetylation site. Modified Lys 248 was marked in red. L chain: light chain, H chain: heavy chain, M-H chain: modified heavy chain, Atezo: atezolizumab.

FIG. 5A-5B Acetylation of therapeutic antibody and immunofluorescence. (FIG. 5A) Acetylation reaction monitored by SDS-PAGE and fluorescent scanning. Reaction condition: 4 μM antibodies, 24 μM F1, PBS buffer (pH 7.4), at 37° C. for 1 h. The reactions were quenched with 1% SDS and DBCO-TAMRA was added at room temperature for 1.5 h for SPAAC reaction before SDS-PAGE and in-gel fluorescent imaging. Lane M, molecular weight marker; lane 1, M-trastuzumab; lane 2, M-cetuximab; lane 3, M-daratumumab; lane 4-5, reduced results of lane 1-3. (FIG. 5B) Immunofluorescence results observed by a confocal microscope of modified atezolizumab and trastuzumab with PD-L1 positive cells and HER2 positive cells respectively. Incubation conditions: Cells were incubated with modified antibodies (150 nM) in PBS buffer (pH 7.4) at 37° C. for 15 min before the fixation of cells.

FIG. 6A-6C Formation and cell fusion of immunoliposome. (FIG. 6A) Synthesis of the antibody-lipid conjugate. Reaction condition: 10 μM M-Tras, 100 μM DSPE-PEG2000-DBCO, PBS buffer (pH 7.4), at 37° C. for 4 h. The reaction was monitored by SDS-PAGE. (FIG. 6B) Scheme to show the generation of immunoliposome and target cell fusion experiments. (FIG. 6C) Fusion of liposomes with SK-OV-3 cells. Reaction condition: cells were incubated with different liposomes (3.3 μM) in a DMEM medium at 37° C. for 2, 10, 25 min. Cells were fixed after washing and then imaged.

FIG. 7A-7D Tras-atezo bsAbC. (FIG. 7A) Scheme to show the generation of bispecific antibody complexes and the structure of two bifunctional linkers. (FIG. 7B) Two azidoacetyl modified antibodies were conjugated with distinct bifunctional linkers (methyl tetrazine or norboroene) were coincubated in PBS (3 mg/mL) at 37° C. Aliquots were taken at different time points and analyzed by SDS-PAGE. Lane M, molecular weight marker; lane 1, trastuzumab-methyl tetrazine; lane 2, atezolizumab-norbornene; lane 3, anti-PD-L1/anti-HER2 bispecific antibody mixture after 48 h incubation; lane 4-5, reduced results of lane 1-2; lane 6-8, reduced results of bispecific antibody mixture after 24, 48, 72 h incubation. MTz: methyl tetrazine, Nb: norbornene. (FIG. 7C) Representative negative stain EM micrograph. Yellow and cyan arrows indicate tras/atezo and bsAbC, respectively. Scale bar=50 nm. (FIG. 7D) Six representative reference-free class averages and interpretative diagrams of tras/atezo and bsAbC antibodies. Scale bar=10 nm.

FIG. 8A-8C Tras-OKT3 bsABC and T cell activation. (FIG. 8A) Tras-OKT3 bsABC was generated by the same reaction condition as the anti-PD-L1/anti-HER2 bispecific antibody. The result was analyzed by SDS-PAGE. Lane M, molecular weight marker; lane 1, trastuzumab-methyl tetrazine; lane 2, OKT3-norbornene; lane 3, anti-HER2/anti-CD3 bispecific antibody mixture after 48 h incubation; lane 4-6, reduced results of lane 1-3. (FIG. 8B) Fluorescence confocal microscope images of the interaction between SK-OV-3 (green) cells and Jurkat cells (red) in the presence of the anti-HER2/anti-CD3 bispecific antibody mixture. A mixture of unconjugated anti-HER2 antibody and anti-CD3 antibody at a 1:1 ratio was used as a negative control. (FIG. 8C) Cytotoxicity with SK-OV-3/HER2+ cells in the presence of T cells isolated from human PBMCs and bispecific antibody mixture. Anti-HER2 antibody or anti-CD3 antibody were used as negative control. After 48 h of incubation at 37° C. and 5% CO₂, cytotoxicity was measured by amount of LDH (lactate dehydrogenase) release from lysed cells using the CyQUANT™ LDH Cytotoxicity Assay (ThermoFisher). In separate wells, SK-OV-3/HER2+ or MDA-MB-231/HER2− cells with no PBMCs were incubated and lysed using the lysis buffer (provided in the assay kit) as maximum cytotoxicity controls. The absorbance at 490 nm was recorded using a SpectraMax 250 plate reader (Molecular Devices Corp.). Percent cytotoxicity was calculated by % Cytotoxicity=(Absorbanceₑₓₚₜ−Absorbance_{spontaneous average})/(Absorbance_{max average}−Absorbance_{spontaneous average}).

FIG. 9 The co-crystal structure of the Fc-III peptide and IgG Fc. The distances between Fc-III E8 and IgG1 Fc K246, K248 were about 15.1 Å and 5.5 Å, respectively (PDB ID: 1DN2).

FIG. 10 Target selectivity of peptide-driven acetylation towards IgG from different species. Reaction condition: 4 μM protein, 24 μM F1, PBS buffer (pH 7.4), at 37° C. for 1 h. The reactions were quenched with 1% SDS and DBCO-TAMRA was added at room temperature for 1.5 h for copper-free click chemistry. Then the reactions were analyzed by denaturing SDS-PAGE and imaged using in-gel fluorescence scanning and Coomassie blue staining. Each lane contains 6 g of antibodies.

FIG. 11 Specific acetylation of Fc protein in a complex protein mixture. Fc protein was spiked to a mixture of proteins from HeLa cell lysate, and the F1 peptide was added to the lysate to initiate acetylation reaction. Modified Fc region was seen in the fluorescent image (lane 3). lane M, molecular weight marker; lane 1, HeLa cell lysate (20 μg) and DBCO-TAMRA; lane 2, HeLa cell lysate (20 μg), DBCO-TAMRA and F1; lane 3, Fc-spiked (2 μg) HeLa cell lysate (20 μg), DBCO-TAMRA and F1; lane 4, Fc (2 μg), DBCO-TAMRA and F1; lane 5, Fc (2 μg). Reactions were performed in pH 7.4 RIPA lysis buffer at 37° C. for 1 h with a 6-fold excess of peptides, followed by copper-free click chemistry with DBCO-TAMRA. Then the reactions were analyzed by denaturing SDS-PAGE and imaged using in-gel fluorescence scanning and Coomassie blue staining.

FIG. 12A-12B Acetylation of Fc at different reaction conditions. (FIG. 12A) The effect of buffer condition. Reaction condition: 4 μM IgG Fc, 24 μM F1, at 37° C. for 1 h. Lane M, molecular weight marker; lane 1, PBS Buffer (pH 7.4); lane 2, PBS Buffer (pH 7.4); lane 3, RIPA Lysis Buffer (pH 7.4); lane 4, Tris-HCl Buffer (pH 7.4); lane 5, Homogenization Buffer (pH 7.4); lane 6, Citrate Phosphate Buffer (pH 7.4); lane 7, Citrate Phosphate Buffer (pH 6.0); lane 8, Citrate Phosphate Buffer (pH 8.0); lane 9, Borate Buffer (pH 8.0). (FIG. 12B) The effect of temperature. Reaction condition: 4 μM IgG Fc, 24 μM F1, PBS buffer (pH 7.4), for 1 h. The reactions were quenched with 1% SDS and DBCO-TAMRA was added at room temperature for 1.5 h for copper-free click chemistry. Then the reactions were analyzed by denaturing SDS-PAGE, and imaged using in-gel fluorescence scanning and Coomassie blue staining. Protein loading in each lane was 2 μg.

FIG. 13A-13C Binding affinity of peptide f-F0 to atezolizumab measured by microscale thermophoresis (MST). Peptide f-F0 was set as the target at 10 nM, and atezolizumab as ligand to titrate up to 2 μM. Ligand-induced fluorescence change was used to determine the K_{d} value. (FIG. 13A) Dose response curve. (FIG. 13B) Capillary scans. (FIG. 13C) MST traces. The obtained K_{d} value was consistent with previous reports.^{1,2}

FIG. 14A-14C Binding affinity of peptide f-F4 to atezolizumab measured by MST. Peptide f-F4 was set as the target at 50 nM, and atezolizumab as ligand to titrate up to 16 μM. Ligand-induced fluorescence change was used to determine the K_{d} value. (FIG. 14A) Dose response curve. (FIG. 14B) Capillary scans. (FIG. 14C) MST traces.

FIG. 15A-15C Binding affinity of peptide f-F5 to atezolizumab measured by MST. Peptide f-F5 was set as the target at 50 nM, and atezolizumab as ligand to titrate up to 16 μM. Ligand-induced fluorescence change was used to determine the K_{d} value. (FIG. 15A) Dose response curve. (FIG. 15B) Capillary scans. (FIG. 15C) MST traces.

FIG. 16A-16B Studies of the binding of M-Atezo to PD-L1. (FIG. 16A) A pull-down experiment in vitro. (left) M-Atezo reserved the binding ability to PD-L1 indicated by the eluted protein band. An irrelevant IgG did not bind with PD-L1 immobilized resins. (right) Competitive binding between M-Atezo and Atezo for PD-L1. No significant decrease of the amount of eluted M-Atezo was observed in the in-gel fluorescence image when incubating M-Atezo and Atezo at a ratio of 1:1 with PD-L1 loaded resins, which suggested that M-Atezo bind with PD-L1 with efficacy comparable to Atezo. (FIG. 16B) FITC labeled PD-L1 was immobilized on Ni-NTA agarose resin through polyhistidine tag. DBCO-TAMRA tagged atezolizumab, but not a control IgG, bound with PD-L1 resin. Atezo: Atezolizumab, M-Atezo: modified atezolizumab.

FIG. 17 MALDI-TOF MS analysis of acylated Fc protein. Peptide fragments of Fc and acylated Fc (M-Fc) were produced by tryptic digestion. In the high-m/z region, a new peak corresponding to the modified peptide fragment matching the sequence of TCPPCPAPELLGGPSVFLFPPKPKDTLMISR (SEQ ID NO: 1) appeared. Lys 248 was marked in red.

FIG. 18 MALDI-TOF MS analysis of acylated native human IgG. Peptide fragments of IgG heavy chain (H) and acylated IgG heavy chain (M-H) were generated by tryptic digestion. In the high-m/z region, a new peak corresponding to the acylated peptide fragment matching the sequence of THTCPPCPAPELLGGPSVFLFPPKPKDTLMISR (SEQ ID NO: 2) appeared.

FIG. 19 MALDI-TOF MS analysis of acylated atezolizumab and the assignment of peptide fragments. Observed peptide fragments were shown in blue.

FIG. 20 MALDI-TOF MS analysis to prove the acetylation site. Two peaks ended with Lys 248 disappeared (in yellow and green areas) with a concomitant appearance of new peaks (in blue area) following acetylation reaction.

FIG. 21 LC-MS/MS analysis of peptide fragments from heavy chain of atezolizumab after tryptic digestion.

FIG. 22 LC-MS/MS analysis of peptide fragments from acylated heavy chain of atezolizumab after tryptic digestion. New peaks with m/z of 936.66729 and 939.85957 were observed. These two peaks were consistent with the calculated molecular weight of modified peptide fragment with a sequence of THTCPPCPAPELLGGPSVFLFPPKPKDTLMISR (SEQ ID NO: 2) with or without methionine oxidation.

FIG. 23 Identifying acetylation site on atezolizumab by LC-MS/MS analysis on the peak with m/z of 936.66729.

FIG. 24 Identifying acetylation site on atezolizumab by LC-MS/MS analysis on the peak with m/z of 939.85957.

FIG. 25A-25B HPLC purification of the acylated peptide from tryptic digestion product of acylated atezolizumab. The peptide fragments were analyzed by reverse phase HPLC using C18 column (Hypersil GOLD column, Thermo Scientific). The peak with an absorption in 555 nm was collected.

FIG. 26 MS/MS analysis of the peptide collected from FIG. 26. Two peaks with m/z of 936.66817 and 936.66817 were identified, matching the theoretical molecular weight of the peptide with or without methionine oxidation. MS/MS pattern of the peak with m/z of 936.66817 was identical as that in FIG. 24.

FIG. 27A-27B The study of IgG Fc acetylation reaction with different payloads. (FIG. 27A) A set of reactive peptides with different acyl groups tested for labeling efficiency. (FIG. 27B) The yields of IgG Fc labeled by peptides at 37° C. Reaction condition: 4 μM IgG Fc, 24 μM peptide, in PBS buffer (pH 7.4) for 2 h or in borate buffer (pH 8.5) for 15 h, respectively. The labeling efficiency of F1 is not shown in pH 8.5 because peptide F1 is extremely unstable at basic condition. The yields were quantified by MS analysis with in-solution Glu-C digestion of modified Fc or by in-gel fluorescent image (FIGS. 27A-27B, 28-16).

FIG. 28 MALDI-TOF MS analysis of Fc acetylation by F7. (left) Complete diagram. (right) Partial enlarged detail. The 42-Da mass shift due to acetylation is shown. Reaction condition: 4 μM IgG Fe, 24 μM peptide, 37° C., in PBS buffer (pH 7.4) for 2 h or in borate buffer (pH 8.5) for 15 h. The reactions (5 μg Fc) were purified by HPLC with C4 column and analyzed by MALDI-TOF MS.

FIG. 29 MALDI-TOF MS analysis of Fc acetylation by F8. (left) Complete diagram. (right) Partial enlarged detail. The 80-Da mass shift due to acetylation is shown. Reaction condition: 4 μM IgG Fe, 24 μM peptide, 37° C., in PBS buffer (pH 7.4) for 2 h or in borate buffer (pH 8.5) for 15 h. The reactions (5 μg Fc) were purified by HPLC with C4 column and analyzed by MALDI-TOF MS.

FIG. 30 MALDI-TOF MS analysis of Fc biotinylation by F9. (left) Complete diagram. (right) Partial enlarged detail. The 226-Da mass shift due to biotinylation is shown. Reaction condition: 4 μM IgG Fe, 24 μM peptide, 37° C., in PBS buffer (pH 7.4) for 2 h or in borate buffer (pH 8.5) for 15 h. The reactions (5 μg Fc) were purified by HPLC with C4 column and analyzed by MALDI-TOF MS.

FIG. 31 MALDI-TOF MS analysis of Fc acetylation by F10. (left) Complete diagram. (right) Partial enlarged detail. No obvious mass shift was observed. Reaction condition: 4 μM IgG Fe, 24 μM peptide, 37° C., in PBS buffer (pH 7.4) for 2 h or in borate buffer (pH 8.5) for 15 h. The reactions (5 μg Fc) were purified by HPLC with C4 column and analyzed by MALDI-TOF MS.

FIG. 32 MALDI-TOF MS analysis of Fc acetylation by F11. (left) Complete diagram. (right) Partial enlarged detail. No obvious mass shift was observed. Reaction condition: 4 μM IgG Fe, 24 μM peptide, 37° C., in PBS buffer (pH 7.4) for 2 h or in borate buffer (pH 8.5) for 15 h. The reactions (5 μg Fc) were purified by HPLC with C4 column and analyzed by MALDI-TOF MS.

FIG. 33 MS analysis of peptide fragments from Fc and acetylated Fc with in-solution Glu-C digestion. A new peak was observed with m/z 2784.1, which was consistent with the calculated molecular weight of modified peptide fragment with a sequence of LLGGPSVFLFPPKPKDTLMISRTPE (SEQ ID NO: 3). The yields of the labeled Fc were about 41% in PBS buffer (pH 7.4) for 2 h and 90% in borate buffer (pH 8.5) for 15 h, which was quantified by peak area.

FIG. 34 MS analysis of peptide fragments from Fc and acylated Fc with in-solution Glu-C digestion. A new peak was observed with m/z 2821.8, which was consistent with the calculated molecular weight of modified peptide fragment with a sequence of LLGGPSVFLFPPKPKDTLMISRTPE (SEQ ID NO: 3). The yields of the labeled Fc were about 17% in PBS buffer (pH 7.4) for 2 h and 84% in borate buffer (pH 8.5) for 15 h, which was quantified by peak area.

FIG. 35 MS analysis of peptide fragments from Fc and biotinylated Fc with in-solution Glu-C digestion. A new peak was observed with m/z 2968.0, which was consistent with the calculated molecular weight of modified peptide fragment with a sequence of LLGGPSVFLFPPKPKDTLMISRTPE (SEQ ID NO: 3). The yields of the labeled Fc were about 18% in PBS buffer (pH 7.4) for 2 h and 73% in borate buffer (pH 8.5) for 15 h, which was quantified by peak area.

FIG. 36 MS analysis of peptide fragments from Fc and acylated Fc with in-solution Glu-C digestion. A new peak was observed with m/z 2950.2, which was consistent with the calculated molecular weight of modified peptide fragment with a sequence of LLGGPSVFLFPPKPKDTLMISRTPE (SEQ ID NO: 3). The yields of the labeled Fc were about 5% in PBS buffer (pH 7.4) for 2 h and 26% in borate buffer (pH 8.5) for 15 h, which was quantified by peak area.

FIG. 37 MS and SDS-PAGE analysis of Fc acetylation by peptide F11. (left) MALDI-TOF MS analysis of peptide fragments from Fc and modified Fc with in-solution Glu-C digestion. No obvious new peak was observed. (right) The comparison between Fc modification by F1 and Fc modification by F11, which was characterized by the Coomassie-stained gel and fluorescent image. The yields of the labeled Fc were about 5% in PBS buffer (pH 7.4) for 2 h and 13% in borate buffer (pH 8.5) for 15 h, which was quantified by ImageJ based on the intensity of bands in fluorescent image.

FIG. 38A-38B Characterization of liposome by dynamic light scattering (DLS) Liposomes were composed of 1-palmitoyl-2-oleoyl-glycero-3-phosphocholine (POPC), cholesterol and L-α-Phosphatidylethanolamine-N-(7-nitro-2-1,3-benzoxadiazol-4-yl) (NBD PE) in a molar ratio of 67:30:3. All lipids were dissolved in chloroform and evaporated under reduced pressure to form lipid films on the flask wall. The lipid films were then re-suspended in PBS buffer. After extrusion of liposomes with ten cycles through a pore size (100 nm) polycarbonate filter, the liposome was incubated with lipid-Tras at 60° C. at a ratio of 50 μg antibody/mol lipid. The mixture was incubated for 30 min with slow agitation. DLS were tested for characterization of liposome (FIG. 38A) and immunoliposome (FIG. 38B). There is no significant size change after liposome modification with the antibody. Tras: trastuzumab FIGS. 39A-39B The synthesis of bifunctional linker M-Tz-PEG4-DBCO used for preparing dimerized IgG. (FIG. 39A) Methyltetrazine-amine (1 mM) was incubated with the DBCO-PEG4-NHS ester (1.2 mM) in phosphate buffer (pH 8.2) at RT for 3 h. The reaction mixture was purified by HPLC and lyophilized. (FIG. 39B) QEF MS Analysis of M-Tz-PEG4-DBCO calcd [M+Na]⁺ 758.32815, observed 758.32727.

FIGS. 40A-40B The synthesis of bifunctional linker Nb-PEG4-DBCO used for preparing dimerized IgG. (FIG. 40A) 5-Norbonene-2-methanamine (1 mM) was incubated with the DBCO-PEG4-NHS ester (1.2 mM) in phosphate buffer (pH 8.2) at RT for 3 h. The reaction mixture was purified by HPLC and lyophilized. (FIG. 40B) QEF MS Analysis of Nb-PEG4-DBCO calcd [M+Na]⁺ 680.33075, observed 680.33062.

FIG. 41 Synthetic route of F1.

FIGS. 42A-42C Characterization of peptide F1. (FIG. 42A) Chemical structure of F1. (FIG. 42B) MALDI-TOF MS analysis of F1: calc. 1770.8, obs. 1770.8. (FIG. 42C) HPLC analysis of F1.

FIGS. 43A-43C Characterization of peptide F2. (FIG. 43A) Chemical structure of F2. (FIG. 43B) MALDI-TOF MS analysis of F2: calc. 1786.7, obs. 1786.8. (FIG. 43C) HPLC analysis of F2.

FIGS. 44A-44C Characterization of peptide F3. (FIG. 44A) Chemical structure of F3. (FIG. 44B) MALDI-TOF MS analysis of F3: calc. 1762.8, obs. 1762.8. (FIG. 44C) HPLC analysis of F3.

FIGS. 45A-45C Characterization of peptide F6. (FIG. 45A) Chemical structure of F6. (FIG. 45B) MALDI-TOF MS analysis of F6: calc. 1770.8, obs. 1771.0. (FIG. 45C) HPLC analysis of F6.

FIGS. 46A-46C Characterization of peptide f-F0. (FIG. 46A) Chemical structure of f-F0. (FIG. 46B) MALDI-TOF MS analysis of f-F0: calc. 2046.8, obs. 2046.6. (FIG. 46C) HPLC analysis of f-F0.

FIGS. 47A-47B Characterization of peptide f-F4. (FIG. 47A) Chemical structure of f-F4. (FIG. 47B) MALDI-TOF MS analysis of f-F4: calc. 2162.8, obs. 2163.2. (FIG. 47C) HPLC analysis of f-F4.

FIGS. 48A-48C Characterization of peptide f-F5. (FIG. 48A) Chemical structure of f-F5. (FIG. 48B) MALDI-TOF MS analysis of f-F5: calc. 2244.9, obs. 2245.3. (FIG. 48C) HPLC analysis of f-F5.

FIGS. 49A-49C Characterization of peptide F7. (FIG. 49A) Chemical structure of F7. (FIG. 49B) MALDI-TOF MS analysis of F7: calc. 1729.8, obs. 1729.8. (FIG. 49C) HPLC analysis of F7.

FIGS. 50A-50C Characterization of peptide F8. (FIG. 50A) Chemical structure of F8. (FIG. 50B) MALDI-TOF MS analysis of F8: calc. 1767.8, obs. 1767.7. (FIG. 50C) HPLC analysis of F8.

FIGS. 51A-51C Characterization of peptide F9. (FIG. 51A) Chemical structure of F9. (FIG. 51B) MALDI-TOF MS analysis of F9: calc. 1913.8, obs. 1913.9. (FIG. 51C) HPLC analysis of F9.

FIGS. 52A-52C Characterization of peptide F10. (FIG. 52A) Chemical structure of F10. (FIG. 52B) MALDI-TOF MS analysis of F10: calc. 1895.8, obs. 1895.9. (FIG. 52C) HPLC analysis of F10.

FIGS. 53A-53C Characterization of peptide F11. (FIG. 53A) Chemical structure of F11. (FIG. 53B) MALDI-TOF MS analysis of F11: calc. 2099.9, obs. 2100.2. (FIG. 53A) HPLC analysis of F11 at 215 nm and 555 nm, respectively.

### BRIEF DESCRIPTION OF THE SEQUENCES

SEQ ID NO: 1: Fc peptide fragment
SEQ ID NO: 2: IgG peptide fragment
SEQ ID NO: 3: Fc peptide fragment
SEQ ID NO: 4: Fc-III peptide

### DETAILED DISCLOSURE OF THE INVENTION

### Selected Definitions

As used herein, the singular forms “a,” “an,” and “the” are intended to include the plural forms as well, unless the context clearly indicates otherwise. Further, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” “comprise,” include the phrases “consisting essentially of,” “consists essentially of,” “consisting,” and “consists.”

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which will depend in part on how the value is measured or determined, i.e., the limitations of the measurement system. Where particular values are described in the application and claims, unless otherwise stated the term “about” meaning within an acceptable error range for the particular value should be assumed.

In the present disclosure, ranges are stated in shorthand, to avoid having to set out at length and describe each and every value within the range. Any appropriate value within the range can be selected, where appropriate, as the upper value, lower value, or the terminus of the range. For example, a range of 1-10 represents the terminal values of 1 and 10, as well as the intermediate values of 2, 3, 4, 5, 6, 7, 8, 9, and all intermediate ranges encompassed within 1-10, such as 2-5, 2-8, and 7-10. Also, when ranges are used herein, combinations and sub-combinations of ranges (e.g., subranges within the disclosed range) and specific embodiments therein are intended to be explicitly included.

The term “antibody” as used herein refers to a polypeptide encoded by an immunoglobulin gene or immunoglobulin genes, or fragments thereof, which specifically bind and recognize an analyte (antigen). The recognized immunoglobulin light chains are classified as either kappa or lambda. Immunoglobulin heavy chains are classified as gamma, mu, alpha, delta, or epsilon, which in turn define the immunoglobulin classes, IgG, IgM, IgA, IgD and IgE, respectively. An example of a structural unit of immunoglobulin G (IgG antibody) is a tetramer. Each such tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains, respectively.

By “constant region” of an antibody as defined herein is meant the region of the antibody that is encoded by one of the light or heavy chain immunoglobulin constant region genes. By “constant light chain” or “light chain constant region” as used herein is meant the region of an antibody encoded by the kappa or lambda light chains. The constant light chain typically comprises a single domain, and as defined herein refers to positions 108-214 of kappa, or lambda, wherein numbering is according to the EU index (Kabat et al., 1991, Sequences of Proteins of Immunological Interest, 5th Ed., United States Public Health Service, National Institutes of Health, Bethesda). By “constant heavy chain” or “heavy chain constant region” as used herein is meant the region of an antibody encoded by the mu, delta, gamma, alpha, or epsilon genes to define the antibody's isotype as IgM, IgD, IgG, IgA, or IgE, respectively. For full length IgG antibodies, the constant heavy chain, as defined herein, refers to the N-terminus of the CH1 domain to the C-terminus of the CH3 domain, thus comprising positions 118-447, wherein numbering is according to the EU index.

By “Fab” or “Fab region” as used herein is meant the polypeptide that comprises the VH, CH1, VL, and CL immunoglobulin domains. Fab may refer to this region in isolation, or this region in the context of a full-length antibody, antibody fragment or Fab fusion protein, or any other antibody embodiments as outlined herein.

By “Fv” or “Fv fragment” or “Fv region” as used herein is meant a polypeptide that comprises the VL and VH domains of a single antibody.

By “Fc” or “Fc region”, as used herein is meant the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cγ2 and Cγ3 and the hinge between Cγ1 and Cγ2. Although the boundaries of the Fc region may vary, the human IgG heavy chain Fc region is usually defined to comprise residues C226, P230 or A231 to its carboxyl-terminus, wherein the numbering is according to the EU index. Fc may refer to this region in isolation, or this region in the context of an Fc polypeptide, as described below. By “Fc polypeptide” as used herein is meant a polypeptide that comprises all or part of an Fc region. Fc polypeptides include antibodies, Fc fusions, isolated Fcs, and Fc fragments.

By “full length antibody” as used herein is meant the structure that constitutes the natural biological form of an antibody, including variable and constant regions. For example, in most mammals, including humans and mice, the full-length antibody of the IgG isotype is a tetramer and consists of two identical pairs of two immunoglobulin chains, each pair having one light and one heavy chain, each light chain comprising immunoglobulin domains VL and CL, and each heavy chain comprising immunoglobulin domains VH, Cγ1, Cγ2, and Cγ3. In some mammals, for example in camels and llamas, IgG antibodies may consist of only two heavy chains, each heavy chain comprising a variable domain attached to the Fc region.

By “variable region” as used herein is meant the region of an antibody that comprises one or more Ig domains substantially encoded by any of the VL (including Vkappa and Vlambda) and/or VH genes that make up the light chain (including kappa and lambda) and heavy chain immunoglobulin genetic loci respectively. A light or heavy chain variable region (VL and VH) consists of a “framework” or “FR” region interrupted by three hypervariable regions referred to as “complementarity determining regions” or “CDRs”. The extent of the framework region and CDRs have been precisely defined, for example as in Kabat et al. (see “Sequences of Proteins of Immunological Interest,” E. Kabat et al., U.S. Department of Health and Human Services, (1983)), and as in Chothia. The framework regions of an antibody, that is the combined framework regions of the constituent light and heavy chains, serves to position and align the CDRs, which are primarily responsible for binding to an antigen.

By “amino acid modification” herein is meant an amino acid substitution, insertion, and/or deletion in a polypeptide sequence. The preferred amino acid modification herein is a substitution. By “amino acid modification” herein is meant an amino acid substitution, insertion, and/or deletion in a polypeptide sequence. By “amino acid substitution” or “substitution” herein is meant the replacement of an amino acid at a given position in a protein sequence with another amino acid. For example, the substitution Y50W refers to a variant of a parent polypeptide, in which the tyrosine at position 50 is replaced with tryptophan. A “variant” of a polypeptide refers to a polypeptide having an amino acid sequence that is substantially identical to a reference polypeptide, typically a native or “parent” polypeptide. The polypeptide variant may possess one or more amino acid substitutions, deletions, and/or insertions at certain positions within the native amino acid sequence.

“Conservative” amino acid substitutions are those in which an amino acid residue is replaced with an amino acid residue having a side chain with similar physicochemical properties. Families of amino acid residues having similar side chains are known in the art, and include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

Antibodies exist as intact immunoglobulins or as well-characterized fragments produced by digestion of intact immunoglobulins with various peptidases. Thus, for example, pepsin digests an antibody near the disulfide linkages in the hinge region to produce F(ab′)₂, a dimer of Fab which itself is a light chain joined to VH-CH1 by a disulfide bond. The F(ab′)₂ dimer can be reduced under mild conditions to break the disulfide linkage in the hinge region, thereby converting the F(ab′)₂ dimer into two Fab′ monomers. The Fab′ monomer is essentially an Fab with part of the hinge region (see, Paul (Ed.), Fundamental Immunology, Third Edition, Raven Press, N.Y. (1993)). While various antibody fragments are defined in terms of the digestion of an intact antibody, one of skill will appreciate that such fragments may be synthesized de novo either chemically or by utilizing recombinant DNA methodology. Thus, the term “antibody,” as used herein, also includes antibody fragments either produced by the modification of whole antibodies.

Antibodies are commonly referred to according their targets. While the nomenclature varies, one of skill in the art will be familiar and understand that several names can be applied to the same antibody. For example, an antibody specific for IgG can be called “anti-IgG,” “IgG antibody,” “anti-IgG antibody,” etc.

The terms “specific for,”, “specific to”, “specifically binds,” and grammatically equivalent terms refer to a molecule (e.g., antibody or antibody fragment) that binds to its target with at least 2-fold greater affinity than non-target compounds, e.g., at least any of 4-fold, 5-fold, 6-fold, 7-fold, 8-fold, 9-fold, 10-fold, 20-fold, 25-fold, 50-fold, or 100-fold greater affinity. For example, antibodies that specifically binds a given antibody target will typically bind the antibody target with at least a 2-fold greater affinity than a non-antibody target. Specificity can be determined using standard methods, e.g., solid-phase ELISA immunoassays (see, e.g., Harlow & Lane, Using Antibodies, A Laboratory Manual (1998) for a description of immunoassay formats and conditions that can be used to determine specific immunoreactivity).

The term “binds” with respect to an antibody target (e.g., antigen, analyte), typically indicates that an antibody binds a majority of the antibody targets in a pure population (assuming appropriate molar ratios). For example, an antibody that binds a given antibody target typically binds to at least ⅔ of the antibody targets in a solution (e.g., at least any of 75, 80, 85, 90, 91, 92, 93, 94, 95, 96, 97, 98, 99, or 100%). One of skill will recognize that some variability will arise depending on the method and/or threshold of determining binding.

In certain embodiments of the invention a subject is a mammal. Non-limiting examples of a mammal treatable according to the methods of the current invention include mouse, rat, dog, guinea pig, cow, horse, cat, rabbit, pig, monkey, ape, chimpanzee, and human. Additional examples of mammals treatable with the methods of the current invention are well known to a person of ordinary skill in the art and such embodiments are within the purview of the current invention.

For the purposes of this invention the terms “treatment, treating, treat” or equivalents of these terms refer to healing, alleviating, relieving, altering, remedying, ameliorating, improving, or affecting the condition or the symptoms of a subject suffering with a disease or condition, for example, a cancer or an infection. The subject to be treated can be suffering from or at risk of developing the disorder or condition, for example, cancer. When provided therapeutically, the compound can be provided before the onset of a symptom. The therapeutic administration of the substance serves to attenuate any actual symptom.

For the purposes of this invention, the terms “preventing, preventive, prophylactic” or equivalents of these terms are indicate that the compounds of the subject invention are provided in advance of any disease symptoms and are a separate aspect of the invention (i.e., an aspect of the invention that is distinct from aspects related to the terms “treatment, treating, treat” or equivalents of these terms which refer to healing, alleviating, relieving, altering, remedying, ameliorating, improving, or affecting the condition or the symptoms of a subject suffering from cancer). The prophylactic administration of the compounds of the subject invention serves to prevent, reduce the likelihood, or attenuate one or more subsequent symptoms or condition.

By “therapeutically effective dose,” “therapeutically effective amount”, or “effective amount” is intended to be an amount of a compounds of the subject invention disclosed herein that, when administered to a subject, decreases the number or severity of symptoms or inhibits or eliminates the progression or initiation of cancer or reduces any increase in symptoms, or improve the clinical course of the disease as compared to untreated subjects. “Positive therapeutic response” refers to, for example, improving the condition of at least one of the symptoms of cancer.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual. Generally, the dosage of the compounds of the subject invention will vary depending upon such factors as the patient's age, weight, height, sex, general medical condition and previous medical history.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, 50, 75, 100, 150, 200, 250, 300, 350, 400, 450, 500, 600, 700, 800, 900, 1000 or more therapeutically effective doses of a composition comprising the compounds of the subject invention as described herein. In some embodiments, doses are administered over the course of 1 day, 2 days, 3 days, 4 days, 5 days, 6 days, 7 days, 10 days, 14 days, 21 days, 30 days, 2 months, 3 months, 6 months, 1 year, 2 years, 3 years, 4 years, 5 years, 6 years, 7 years, 8 years, 9 years, 10 years, or more than 10 years. The frequency and duration of administration of multiple doses of the compositions is such as to inhibit or delay the initiation of cancer. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the results of diagnostic methods for detecting a tumor known in the art. In some embodiments of the invention, the method comprises administration of the compounds at a single time per day or several times per day, including but not limiting to 2 times per day, 3 times per day, and 4 times per day.

As used herein, the term “cancer” refers to the presence of cells possessing abnormal growth characteristics, such as uncontrolled proliferation, immortality, metastatic potential, rapid growth and proliferation rate, perturbed oncogenic signaling, and certain characteristic morphological features.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

### Method of Producing Peptides

In certain embodiments, a Fc binding peptide can be synthesized with 1, 2, 3, 4, 5, 6, 7, 8, 9, 10, or more modified amino acid residues. In preferred embodiments, the modified Fc binding peptide can be derived from Fc-III. In certain embodiments, the modified peptide has 1 modified residue, preferably 1 substituted residue. In preferred embodiments, the His5, Lys6, or Glu8 of the Fc-III peptide can be substituted with a glutamine derivative that contains a phenyl azidoacetate motif at the side chain, to yield synthesized peptides, specifically, F1 (according to formula (I)), F2 (according to formula (II)), F3 (according to formula (III)), F6 (according to formula (IV)), f-F0 (according to formula (V)), f-f4 (according to formula (VI)), f-F5 (according to formula (VII)), F7 (according to formula (VIII)), F8 (according to formula (IX)), F9 (according to formula (X)), F10 (according to formula (XI)), or F11 (according to formula (XII)) peptides:

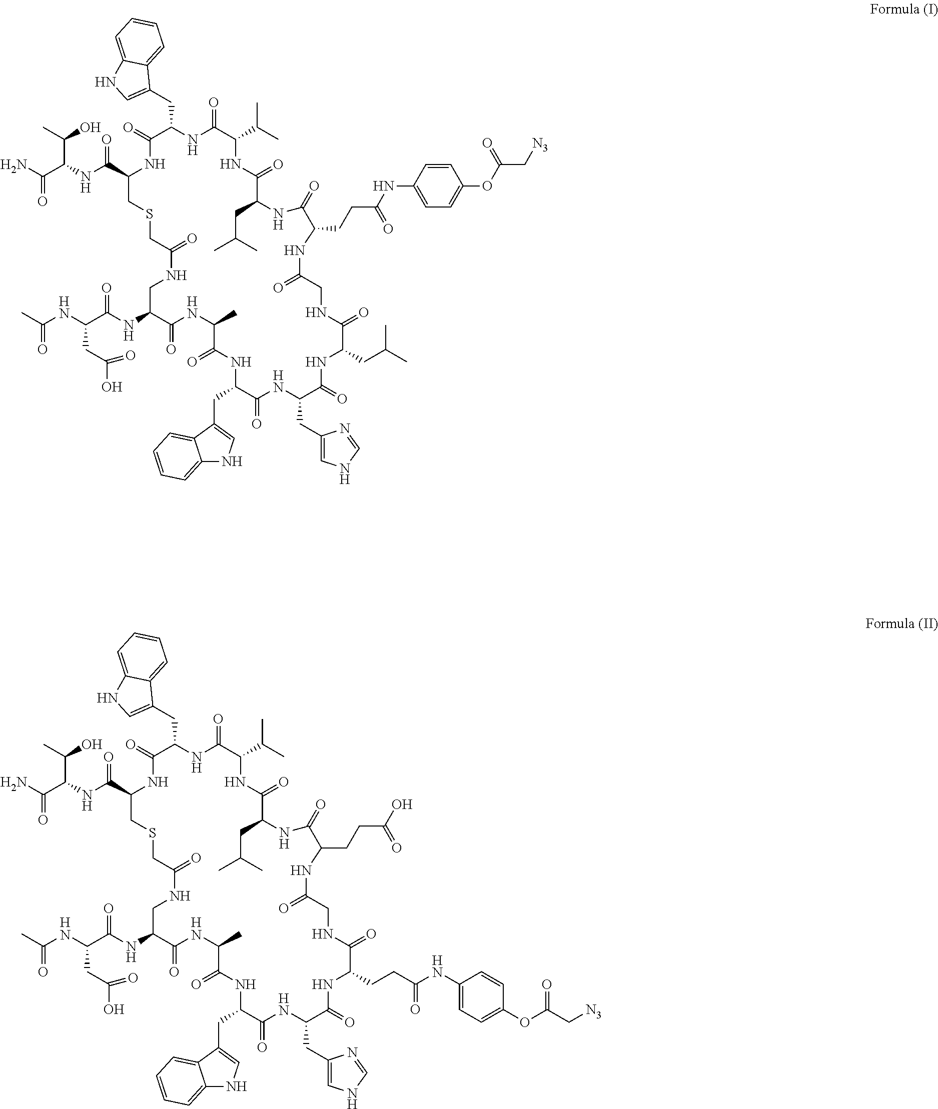

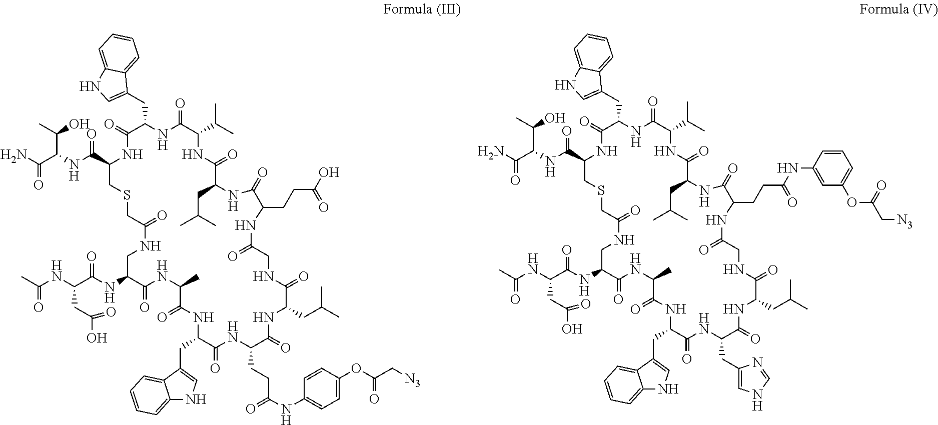

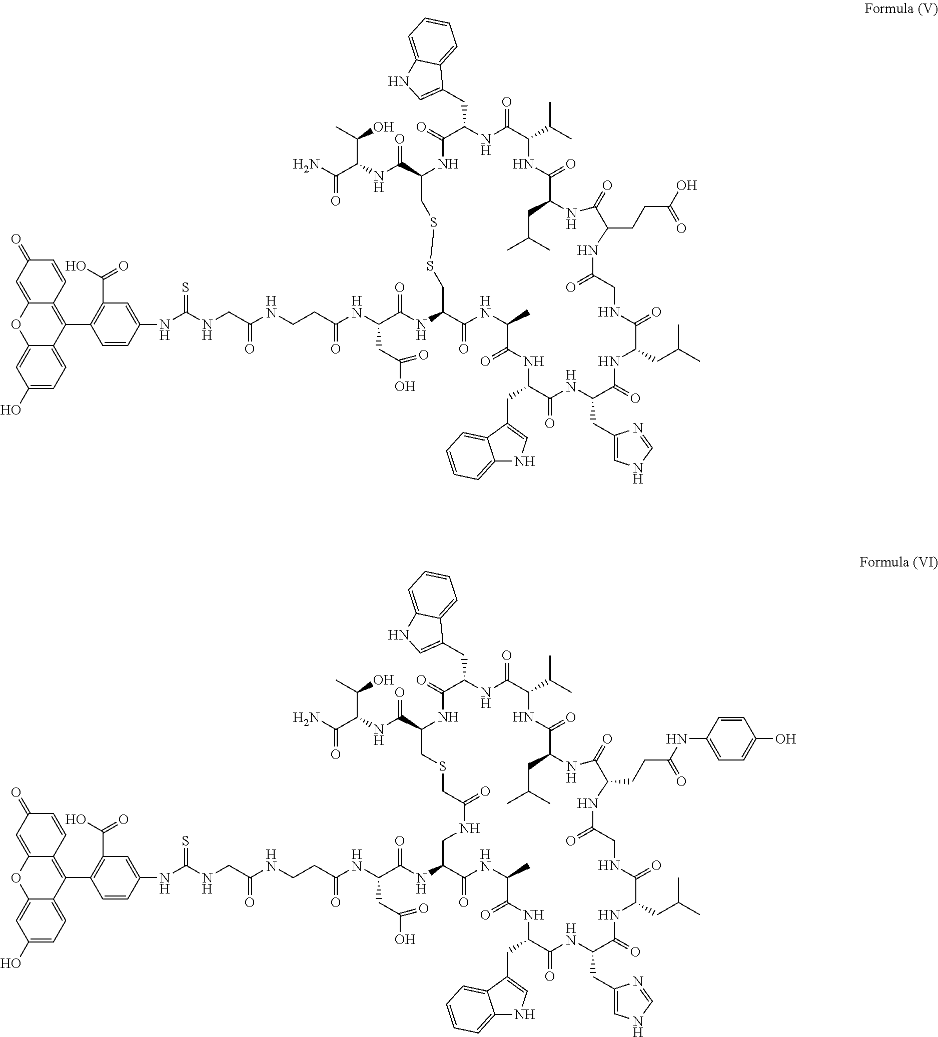

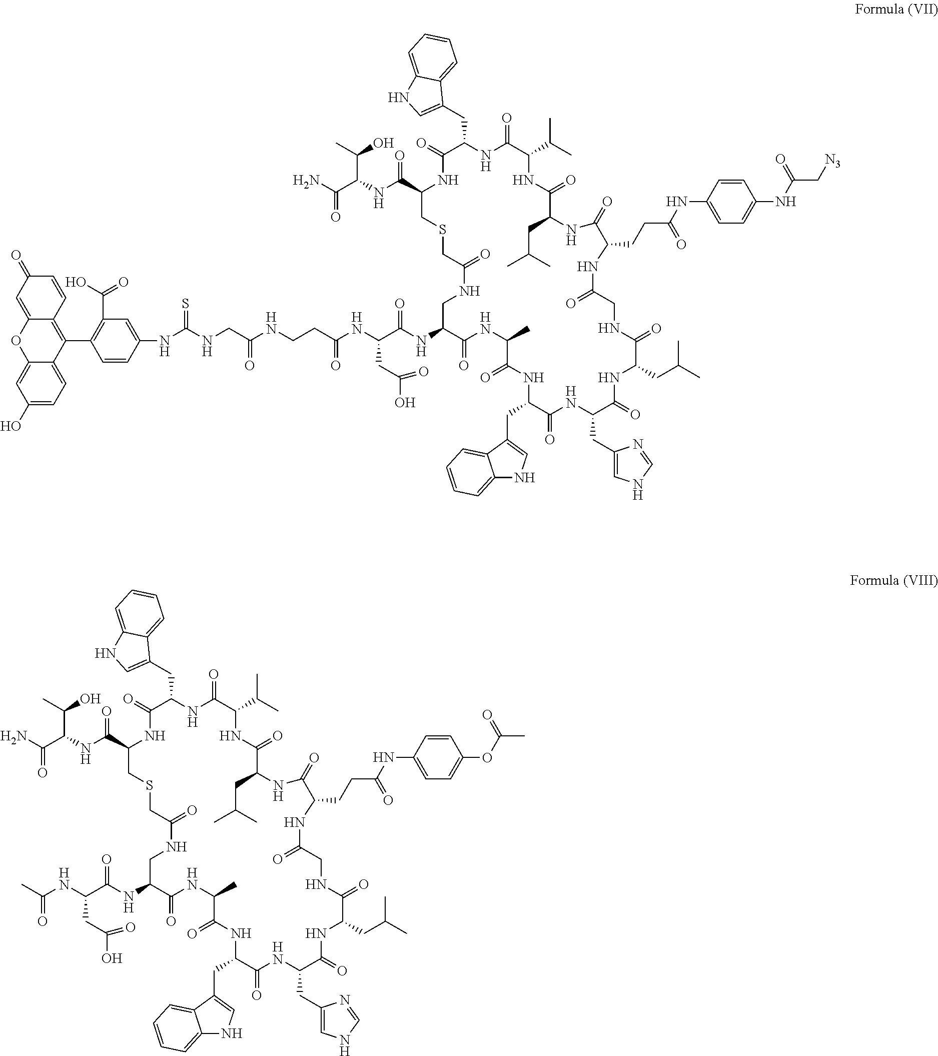

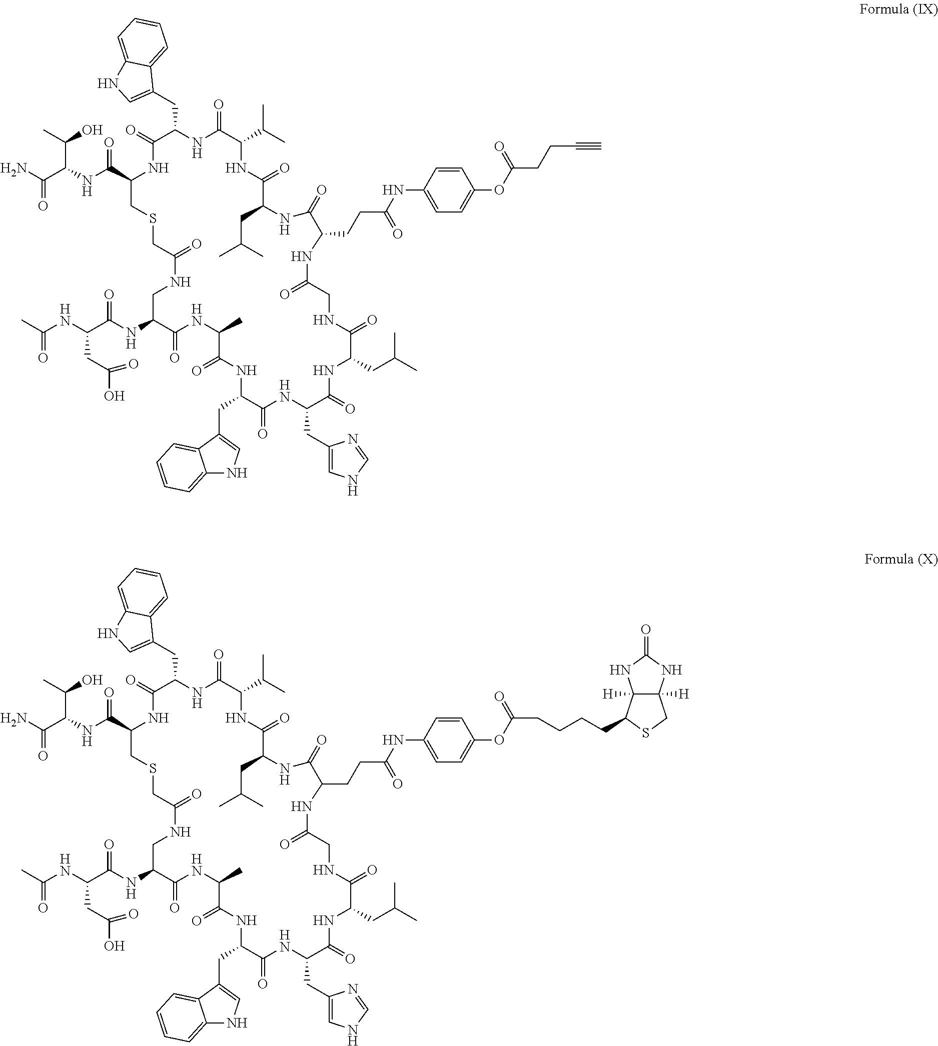

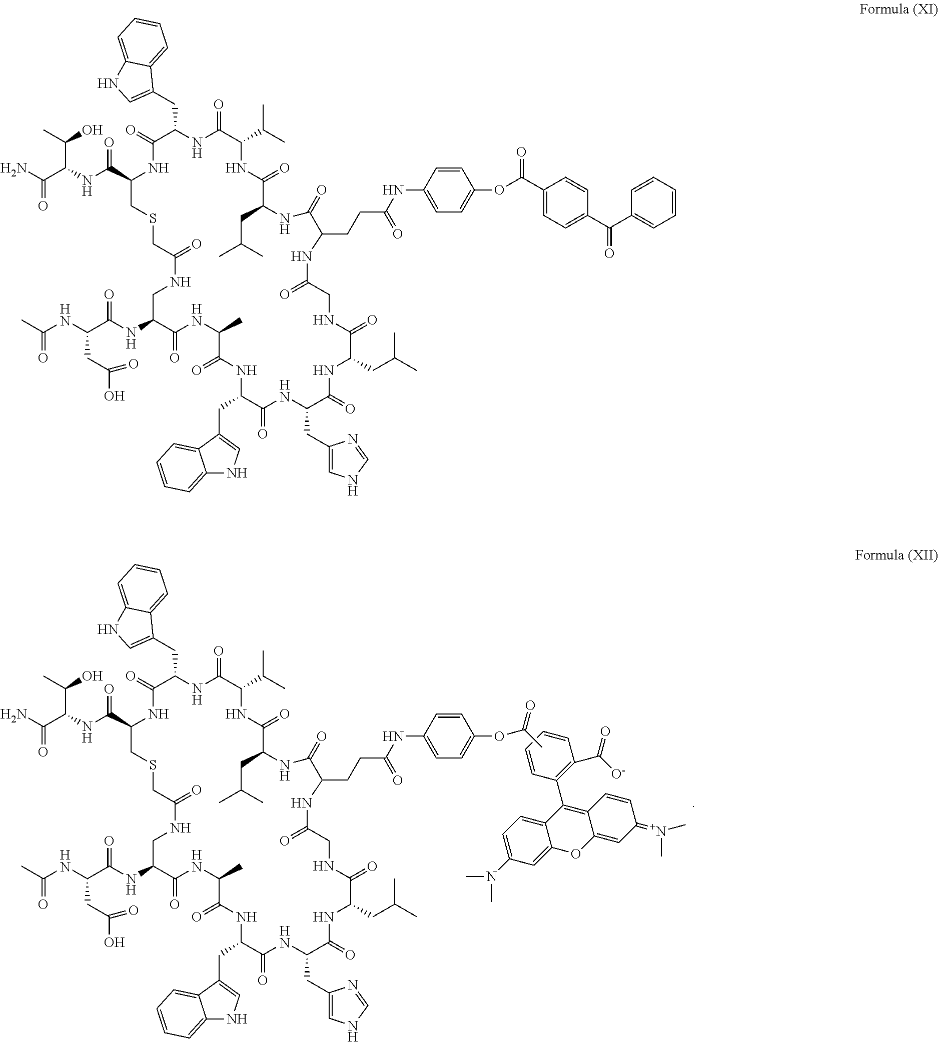

In certain embodiments, the peptides can be synthesized using fluorenylmethoxycarbonyl protecting group-solid phase peptide synthesis (Fmoc-SPPS), which is a well-known method of synthesizing modified peptides. An exemplary method of synthesizing the modified peptide, is discussed below; however, other methods of peptide synthesis can also be used.

In certain embodiments, an azidoacetyl group can be transferred from the modified peptide to an immunoglobulin or fragment thereof, preferably IgG Fc, by a spontaneous acetylation reaction. In certain embodiments, the reaction can occur in phosphate-buffered solution (PBS), or an alternative buffer solution at about 30° C. to about 40° C. or about 37° C. for about 1 min to about 6 h or about 1 h. In certain embodiments, the immunoglobulin or fragment thereof including, for example, IgG Fc, can be selected from IgG subtype IgG1, IgG2, IgG3, IgG4, or any combination thereof. In certain embodiments, the IgG Fc can be derived from a mammal, including, for example, a mouse, rabbit, rat, or human. In preferred embodiments, the immunoglobulin or fragment thereof is rabbit or human derived.

### Methods of Producing Acetylated Antibodies

Antibodies may be produced by a variety of techniques known in the art. Typically, they are produced by immunization of a non-human animal, preferably a mouse, with an immunogen comprising a polypeptide, or a fragment or derivative thereof, typically an immunogenic fragment, for which it is desired to obtain antibodies (e.g. a human polypeptide). The step of immunizing a non-human mammal with an antigen may be carried out in any manner well known in the art for stimulating the production of antibodies in a mouse (see, for example, E. Harlow and D. Lane, Antibodies: A Laboratory Manual, Cold Spring Harbor Laboratory Press, Cold Spring Harbor, NY (1988), the entire disclosure of which is herein incorporated by reference). Other protocols may also be used as long as they result in the production of B cells expressing an antibody directed to the antigen used in immunization. Lymphocytes from a non-immunized non-human mammal may also be isolated, grown in vitro, and then exposed to the immunogen in cell culture. The lymphocytes are then harvested and the fusion step described below is carried out. For preferred monoclonal antibodies, the next step is the isolation of splenocytes from the immunized non-human mammal and the subsequent fusion of those splenocytes with an immortalized cell in order to form an antibody-producing hybridoma. The hybridoma colonies are then assayed for the production of antibodies that specifically bind to the polypeptide against which antibodies are desired. The assay is typically a colorimetric ELISA-type assay, although any assay may be employed that can be adapted to the wells that the hybridomas are grown in. Other assays include radioimmunoassays or fluorescence activated cell sorting. The wells positive for the desired antibody production are examined to determine if one or more distinct colonies are present. If more than one colony is present, the cells may be re-cloned and grown to ensure that only a single cell has given rise to the colony producing the desired antibody. After sufficient growth to produce the desired monoclonal antibody, the growth media containing monoclonal antibody (or the ascites fluid) is separated away from the cells and the monoclonal antibody present therein is purified. Purification is typically achieved by gel electrophoresis, dialysis, chromatography using protein A or protein G-Sepharose, or an anti-mouse Ig linked to a solid support such as agarose or Sepharose beads (all described, for example, in the Antibody Purification Handbook, Biosciences, publication No. 18-1037-46, Edition AC, the disclosure of which is hereby incorporated by reference).

Additionally, a wide range of antibodies are available in the scientific and patent literature that are suitable for the compositions and the methods of the subject invention, including DNA and/or amino acid sequences, or from commercial suppliers. Examples of commercially available antibodies include, for example, atezolizumab, trastuzumab, cetuximab, muromonab, or daratumumab.

Antibodies will typically be directed to a single pre-determined antigen. Examples of antibodies include antibodies that recognize an antigen expressed by a target cell that is to be eliminated, for example a proliferating cell or a cell contributing to a pathology. Examples include antibodies that recognize tumor antigens, microbial (e.g. bacterial) antigens or viral antigens. Other examples include antigens present on immune cells or non-immune cells that are contributing to inflammatory or autoimmune disease, including rejection of transplanted tissue (e.g. antigens present on T cells, e.g. Treg cells, CD4 or CD8 T cells).

As used herein, the term “bacterial antigen” includes, but is not limited to, intact, attenuated or killed bacteria, any structural or functional bacterial protein or carbohydrate, or any peptide portion of a bacterial protein of sufficient length (typically about 8 amino acids or longer) to be antigenic. As used herein, the term “viral antigen” includes, but is not limited to, intact, attenuated or killed whole virus, any structural or functional viral protein, or any peptide portion of a viral protein of sufficient length (typically about 8 amino acids or longer) to be antigenic.

As used herein, the terms “cancer antigen” and “tumor antigen” are used interchangeably and refer to antigens that are differentially expressed by cancer cells or are expressed by non-tumoral cells (e.g. immune cells) having a pro-tumoral effect (e.g. an immunosuppressive effect), and can thereby be exploited in order to target cancer cells. Cancer antigens are antigens which can potentially stimulate apparently tumor-specific immune responses. Some of these antigens are encoded, although not necessarily expressed, or expressed at lower levels or less frequently, by normal cells. These antigens can be characterized as those which are normally silent (i.e., not expressed) in normal cells, those that are expressed only at certain stages of differentiation and those that are temporally expressed, such as embryonic and fetal antigens. Other cancer antigens are encoded by mutant cellular genes, such as oncogenes (e.g., activated RAS oncogene), suppressor genes (e.g., mutant p53), fusion proteins resulting from internal deletions or chromosomal translocations. Still other cancer antigens can be encoded by viral genes such as those carried on RNA and DNA tumor viruses. Still other cancer antigens can be expressed on immune cells capable of contributing to or mediating a pro-tumoral effect, e.g. cell that contributes to immune evasion, a monocyte or a macrophage, optionally a suppressor T cell, regulatory T cell, or myeloid-derived suppressor cell.

The cancer antigens are usually normal cell surface antigens which are either over-expressed or expressed at abnormal times, or are expressed by a targeted population of cells. Ideally the target antigen is expressed only on proliferative cells (e.g., tumor cells) or pro-tumoral cells (e.g. immune cells having an immunosuppressive effect), however this is rarely observed in practice. As a result, target antigens are in many cases selected on the basis of differential expression between proliferative/disease tissue and healthy tissue. Example of cancer antigens include: Receptor Tyrosine Kinase-like Orphan Receptor 1 (ROR1), Crypto, CD4, CD20, CD30, CD19, CD38, CD47, Glycoprotein NMB, CanAg, Her2 (ErbB2/Neu), a Siglec family member, for example CD22 (Siglec2) or CD33 (Siglec3), CD79, CD138, CD171, PSCA, L1-CAM, PSMA (prostate specific membrane antigen), BCMA, CD52, CD56, CD80, CD70, E-selectin, EphB2, Melanotransferrin, Mud 6 and TMEFF2. Examples of cancer antigens also include Immunoglobulin superfamily (IgSF) such as cytokine receptors, Killer-Ig Like Receptor, CD28 family proteins, for example, Killer-Ig Like Receptor 3DL2 (KIR3DL2), B7-H3, B7-H4, B7-H6, PD-L1, IL-6 receptor. Examples also include MAGE, MART-1/Melan-A, gp100, major histocompatibility complex class I-related chain A and B polypeptides (MICA and MICB), or optionally an antigen other than MICA and/or MICB, adenosine deaminase-binding protein (ADAbp), cyclophilin b, colorectal associated antigen (CRC)-C017-1A/GA733, protein tyrosine kinase 7 (PTK7), receptor protein tyrosine kinase 3 (TYRO-3), nectins (e.g. nectin-4), proteins of the UL16-binding protein (ULBP) family, proteins of the retinoic acid early transcript-1 (RAET1) family, carcinoembryonic antigen (CEA) and its immunogenic epitopes CAP-1 and CAP-2, etv6, aml1, prostate specific antigen (PSA), T-cell receptor/CD3-zeta chain, MAGE-family of tumor antigens, GAGE-family of tumor antigens, anti-Müllerian hormone Type II receptor, delta-like ligand 4 (DLL4), DR5, ROR1 (also known as Receptor Tyrosine Kinase-Like Orphan Receptor 1 or NTRKR1 (EC 2.7.10.1), BAGE, RAGE, LAGE-1, NAG, GnT-V, MUM-1, CDK4, MUC family, VEGF, VEGF receptors, Angiopoietin-2, PDGF, TGF-alpha, EGF, EGF receptor, members of the human EGF-like receptor family, e.g., HER-2/neu, HER-3, HER-4 or a heterodimeric receptor comprised of at least one HER subunit, gastrin releasing peptide receptor antigen, Muc-1, CA125, integrin receptors, avB3 integrins, α55ß1 integrins, αIIbß3-integrins, PDGF beta receptor, SVE-cadherin, IL-8 receptor, hCG, IL-6 receptor, CSF1R (tumor-associated monocytes and macrophages), α-fetoprotein, E-cadherin, α-catenin, ß-catenin and γ-catenin, p120ctn, PRAME, NY-ESO-1, cdc27, adenomatous polyposis coli protein (APC), fodrin, Connexin 37, Ig-idiotype, p15, gp75, GM2 and GD2 gangliosides, viral products such as human papillomavirus proteins, imp-1, PlA, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2, although this is not intended to be exhaustive.

In preferred embodiments, the antigen of interest is PD-L1, HER2, EGFR, CD8, CD3, or any combination thereof.

In certain embodiments, the constant regions and/or Fc regions of the proteins of the disclosure are of human origin or are humanized (i.e., derived from a non-human species with a sequence that has been modified to increase the similarity to antibodies naturally produced by humans), optionally comprising amino acid sequences partly or fully derived from a human IgG1 isotype, optionally constant regions and/or Fc regions. In one embodiment, a heavy chain is a chimeric heavy chain comprising amino acid sequences derived from two or more human isotypes (e.g. a heavy chain of IgG1 isotype comprising amino acid sequences derived from a human IgG2, IgG3, or IgG4 isotype).

In certain embodiments, methods of acetylating one or more distinct antibodies or antibody derivatives, including, for example, antibody fragment are provided. In certain embodiments, the antibody is incubated with a modified peptide of the subject invention. In certain embodiments, the modified peptide and antibody can be incubated at about 30° C. to about 40° C. or about 37° C. for about 1 min to about 6 h or about 1 h in a buffer solution, such as, for example, PBS. In certain embodiments, only the heavy chain of the antibody is acetylated. In certain embodiments, a single lysine residue of the antibody is acetylated. In preferred embodiments, the lysine reside is Lys248 of IgG Fc. In preferred embodiments, the modified peptide, such as, for example, a peptide derived from Fc-III, has a single amino acid substitution at, for example, the His5, Lys6, or Glu8 of the Fc-III peptide. The modified amino acid residue can be substituted with a glutamine derivative that contains a phenyl azidoacetate motif at the side chain. In preferred embodiments, the modified peptide is F1 (according to formula (I)), F2 (according to formula (II)), F3 (according to formula (III)), F6 (according to formula (IV)), f-F0 (according to formula (V)), f-f4 (according to formula (VI)), f-F5 (according to formula (VII)), F7 (according to formula (VIII)), F8 (according to formula (IX)), F9 (according to formula (X)), F10 (according to formula (XI)), or F11 (according to formula (XII)). In more preferred embodiments, the modified peptide is F1, according to formula (I):

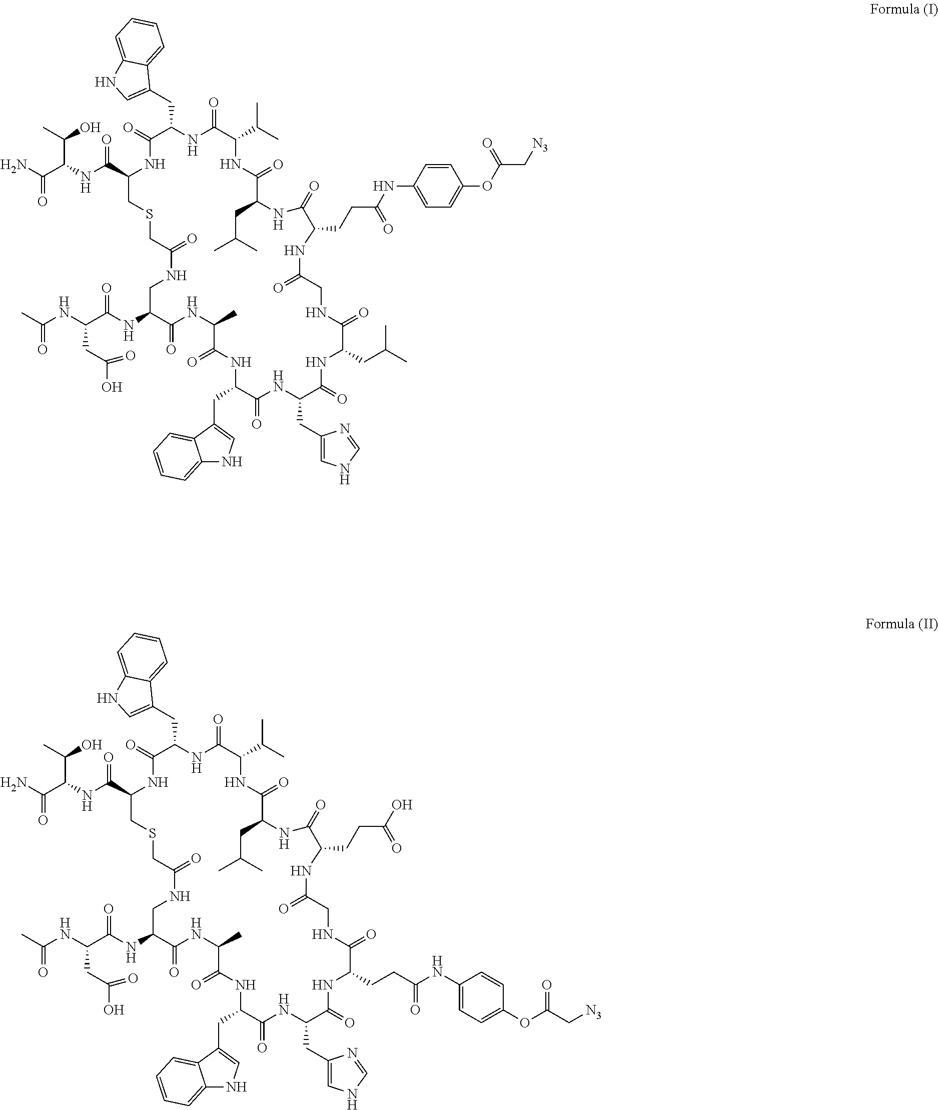

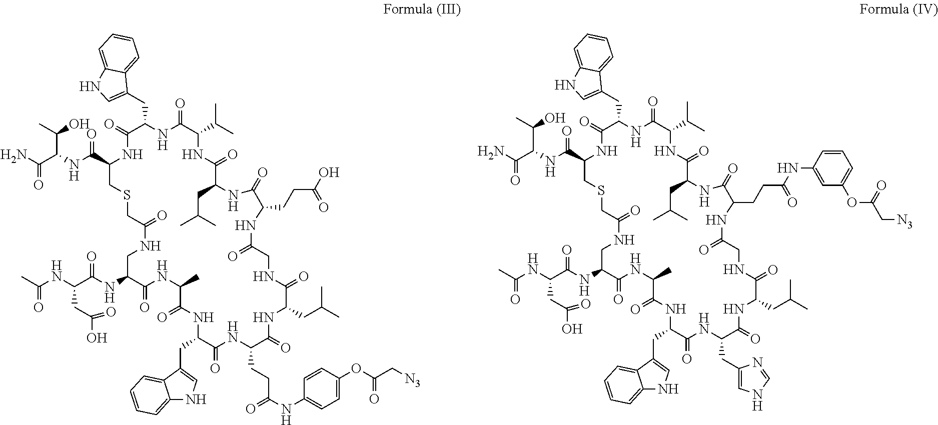

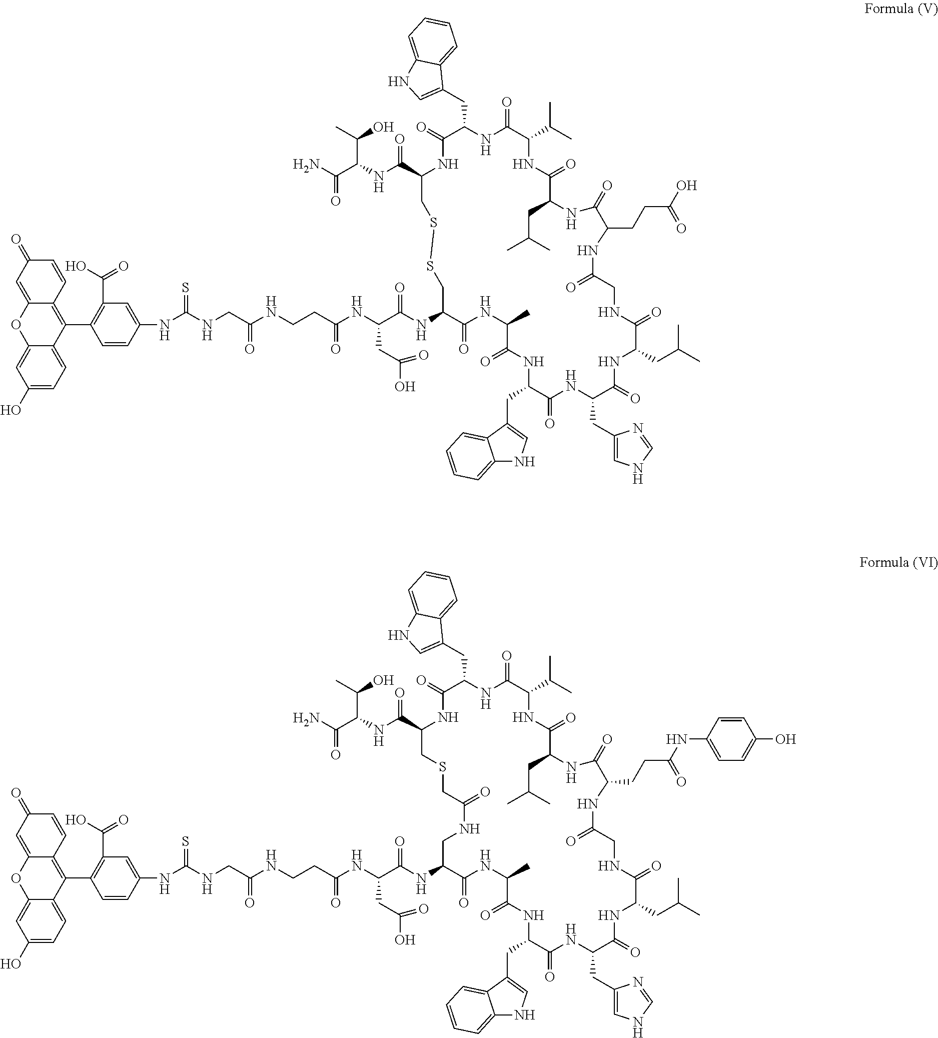

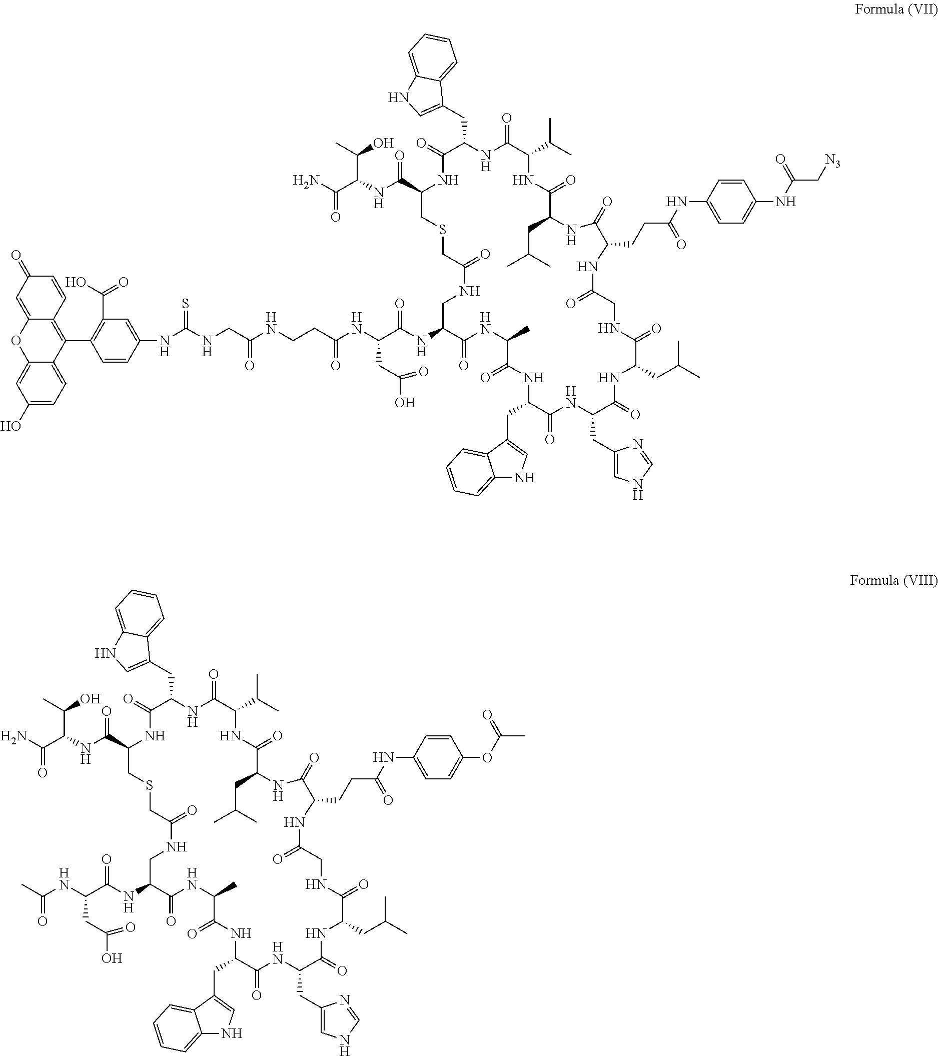

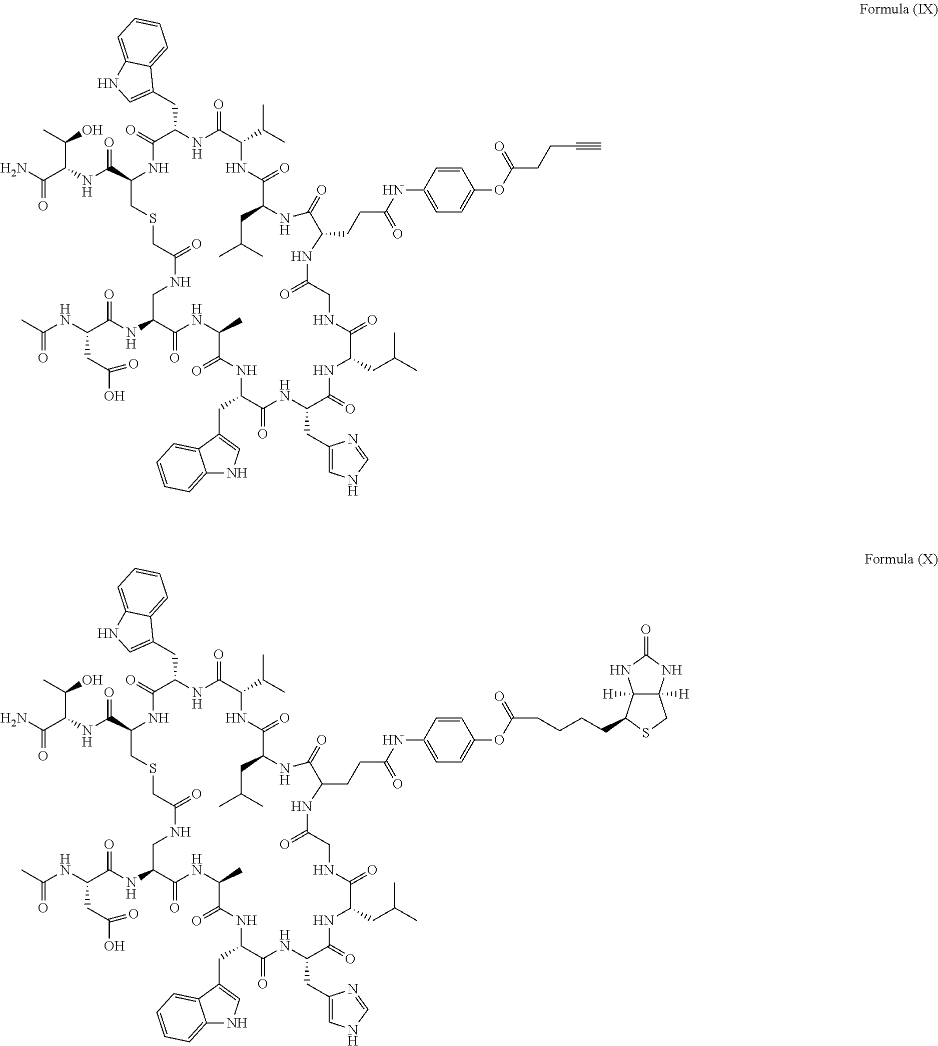

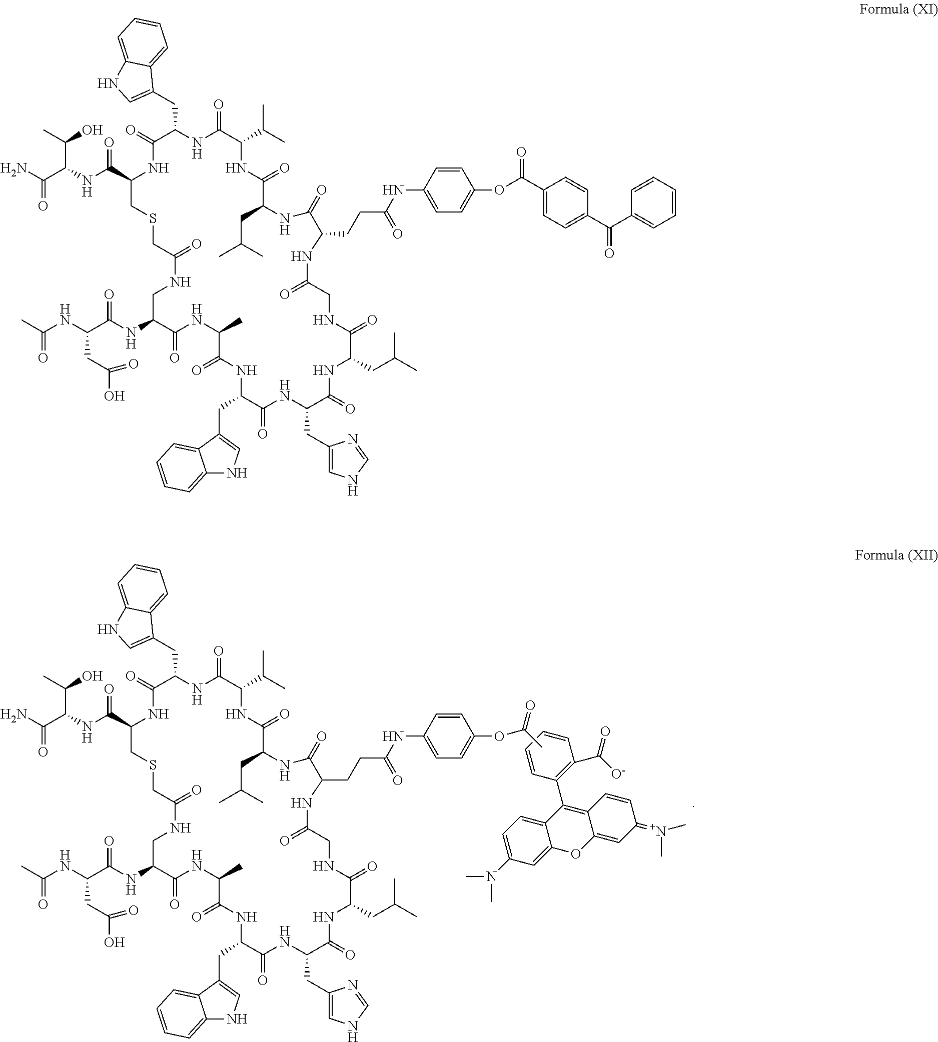

### Methods of Synthesizing Immunoliposomes

In certain embodiments, an acetylated antibody, such as, for example, an azidoacetylated antibody, can be incubated with a functionalized lipid conjugate reagent, such as, for example, DSPE-PEG2000-DBCO for 1 min to about 12 h, about 30 m to about 6 h, or about 2 h. In certain embodiments, at least about 50%, about 60%, about 70%, or about 80% of acetylated antibody can be modified by one lipid molecule.

In certain embodiments, an immunoliposome can be synthesized by incubating the acetylated antibody-DSPE conjugate with a liposome for 1 min to about 12 h, about 55 m to about 6 h, or about 30 min to complete the fusion. In preferred embodiments, the liposomes can be composed of 1-palmitoyl-2-oleoyl-glycero-3-phosphocholine (POPC, Avanti), cholesterol and L-α-Phosphatidylethanolamine-N-(7-nitro-2-1,3-benzoxadiazol-4-yl) (NBD PE, Avanti) in, for example, a molar ratio of 67:30:3.

### Methods of Synthesizing Bispecific Antibody Complexes

In certain embodiments, methods of synthesizing bispecific antibody complexes (bsAbCs) are provided by covalently linking two Fc domains from, for example, different IgGs, such as, for example, a Her2-binding trastuzumab (tras) and a PD-L1-binding atezolizumab (atezo) or Her2-binding trastuzumab and a CD3-binding muromonab (OKT3). First, both antibodies or antibody fragments can be acetylated, preferably, azidoacetylated, as described above to yield, for example, tras-N3 and atezo-N3, respectively.

In certain embodiments, each acetylated antibody or fragments thereof can be functionalized with a distinct linker at a concentration of about 1 μM to about 1000 PM, about 10 μM to about 500 μM, or about 200 μM. Exemplary linkers include, for example, bifunctional linkers DBCO-PEG4-methyl tetrazine (DBCO-PEG4-MTz) and DBCO-PEG4-norborene (DBCO-PEG4-Nb), yielding, for example, tras-MTz and atezo-Nb or tras-MTz and OKT3-Nb.

In certain embodiments, the linker can be synthesized by incubating 5-Norbonene-2-methanamine (1 mM) or methyl-tetrazine-amine with the DBCO-PEG4-NHS ester (about 1 mM to about 10 mM or about 1.2 mM) in phosphate buffer (pH 8.2) at room temperature for about 3 h. In certain embodiments, the reaction mixture can be purified by HPLC and lyophilized.

In certain embodiments, any excessive linker can be removed from the reaction by, for example, a concentrator column. The two antibody-linker conjugates, such as, for example, tras-MTz and atezo-Nb or tras-MTz and OKT3-Nb can be mixed at a 1:1 ratio at about 1 mg/mL to about 5 mg/mL or about 4 mg/mL and incubated at about 30° C. to about 40° C. or about 37° C. for about 1 min to about 6 h or about 3 h for the inverse electron demand Diels-Alder reaction (IEDDA). In certain embodiments, the yield of the bsAbC can be about 50% after about 48 h at room temperature; however a reaction time can be as short as 1 min or can be increased beyond 48 h, such as, for example, 60 h, 72 h, 96 h, 120 h, or longer.

Methods of Using the bsAbCs or Immunoliposomes

The bsAbCs or immunoliposomes can be used for the manufacture of a pharmaceutical preparation and/or for the treatment or diagnosis of a mammal being in need thereof. In one embodiment, provided is the use of any of the methods or any compounds defined above for the manufacture of a pharmaceutical composition and/or for the treatment of a tumor or cancer in a mammal.

The bsAbCs or immunoliposomes may be added to compositions at concentrations of about 0.0001 to about 5% by weight (wt %), preferably about 0.01 to about 0.5 wt %, and most preferably about 0.01% to about 0.05 wt %. In another embodiment, the bsAbCs or immunoliposomes can be in combination with an acceptable carrier and/or excipient, in that the bsAbCs or immunoliposomes may be presented at concentrations of about 0.0001 to about 5% (v/v), preferably, about 0.01 to about 0.5% (v/v), more preferably, about 0.01 to about 0.05% (v/v).

In certain embodiments, anti-cancer therapeutics (i.e., chemotherapeutic agents) can be added to the subject compositions or used in conjunction with the subject compositions, including, for example, doxorubicin. In certain embodiments, the subject compositions can be used before or after surgical removal of the cancerous cells and/or radiation of the cancerous cells.

In one embodiment, the subject compositions are formulated as an orally-consumable product, such as, for example a food item, capsule, pill, or drinkable liquid. An orally deliverable pharmaceutical is any physiologically active substance delivered via initial absorption in the gastrointestinal tract or into the mucus membranes of the mouth. The topic compositions can also be formulated as a solution that can be administered via, for example, injection, which includes intravenously, intraperitoneally, intramuscularly, intrathecally, intracerebroventricularly or subcutaneously. In other embodiments, the subject compositions are formulated to be administered via the skin through a patch or directly onto the skin for local or systemic effects. The compositions can be administered sublingually, buccally, rectally, or vaginally. Furthermore, the compositions can be sprayed into the nose for absorption through the nasal membrane, nebulized, inhaled via the mouth or nose, or administered in the eye or ear.

Orally consumable products according to the invention are any preparations or compositions suitable for consumption, for nutrition, for oral hygiene, or for pleasure, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes). While an orally-deliverable pharmaceutical can be formulated into an orally consumable product, and an orally consumable product can comprise an orally deliverable pharmaceutical, the two terms are not meant to be used interchangeably herein.

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state; the orally consumable products according to the invention therefore also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally-consumable product.

In some embodiments, the orally-consumable product according to the invention can comprise one or more formulations intended for nutrition or pleasure. These particularly include baking products (e.g., bread, dry biscuits, cake, and other pastries), sweets (e.g., chocolates, chocolate bar products, other bar products, fruit gum, coated tablets, hard caramels, toffees and caramels, and chewing gum), alcoholic or non-alcoholic beverages (e.g., cocoa, coffee, green tea, black tea, black or green tea beverages enriched with extracts of green or black tea, Rooibos tea, other herbal teas, fruit-containing lemonades, isotonic beverages, soft drinks, nectars, fruit and vegetable juices, and fruit or vegetable juice preparations), instant beverages (e.g., instant cocoa beverages, instant tea beverages, and instant coffee beverages), meat products (e.g., ham, fresh or raw sausage preparations, and seasoned or marinated fresh meat or salted meat products), eggs or egg products (e.g., dried whole egg, egg white, and egg yolk), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, whey, butter, buttermilk, and partly or wholly hydrolyzed products containing milk proteins), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sauces, and fruit fillings), vegetable preparations (e.g., ketchup, sauces, dried vegetables, deep-freeze vegetables, pre-cooked vegetables, and boiled vegetables), snack articles (e.g., baked or fried potato chips (crisps) or potato dough products and extrudates on the basis of maize or peanuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening or whitening beverages or other food). The present compositions may also serve as semi-finished products for the production of other compositions intended for nutrition or pleasure.

The subject composition can further comprise one or more pharmaceutically acceptable carriers, and/or excipients, and can be formulated into preparations, for example, solid, semi-solid, liquid, or gaseous forms, such as tablets, capsules, powders, granules, ointments, solutions, suppositories, injections, inhalants, and aerosols.

The term “pharmaceutically acceptable” as used herein means compatible with the other ingredients of a pharmaceutical composition and not deleterious to the recipient thereof.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated.

In one embodiment, the compositions of the subject invention can be made into aerosol formulations so that, for example, it can be nebulized or inhaled. Suitable pharmaceutical formulations for administration in the form of aerosols or sprays are, for example, powders, particles, solutions, suspensions or emulsions. Formulations for oral or nasal aerosol or inhalation administration may also be formulated with carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In one embodiment, the compositions of the subject invention can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

In one embodiment, the compositions of the subject invention can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

In certain embodiments, the subject bsAbCs or immunoliposomes can be used in methods of treating cancer, bacterial infections, eukaryotic parasite infections, and/or viral infections. In certain embodiments, the subject bsAbCs or immunoliposomes can engage with T cells of the subject to kill target cancer cells. In certain embodiments, the bsAbC can recruit effector cells to the targeted cancer cells and result in targeted effect-cell mediated cytotoxicity.

Cancers suitable for treatment according to the disclosed methods include, but are not limited to: Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-related cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone cancer, Bone tumor, Breast cancer, Brenner tumor, Bronchial tumor, Bronchioloalveolar carcinoma, Brown tumor, Burkitt's lymphoma, Cancer of unknown primary site, Carcinoid tumor, Carcinoma, Carcinoma in situ, Carcinoma of the penis, Carcinoma of unknown primary site, Carcinosarcoma, Castleman disease, Central nervous system embryonal tumor, Cerebellar astrocytoma, Cerebral astrocytoma, Cervical cancer, Cholangiocarcinoma, Chondroma, Chondrosarcoma, Chordoma, Choriocarcinoma, Choroid plexus papilloma, Chronic lymphocytic leukemia, Chronic monocytic leukemia, Chronic myelogenous leukemia, Chronic myeloproliferative disorder, Chronic neutrophilic leukemia, Clear-cell tumor, Colon cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial uterine cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing family of tumors, Ewing sarcoma, Extracranial germ cell tumor, Extragonadal germ cell tumor, Extrahepatic bile duct cancer, Extramammary Paget's disease, Fallopian tube cancer, Fetus in fetu, Fibroma, Fibrosarcoma, Follicular lymphoma, Follicular thyroid cancer, Gallbladder cancer, Ganglioglioma, Ganglioneuroma, Gastric cancer, Gastric lymphoma, Gastrointestinal cancer, Gastrointestinal carcinoid tumor, Gastrointestinal stromal tumor, Germ cell tumor, Germinoma, Gestational choriocarcinoma, Gestational trophoblastic tumor, Giant cell tumor of bone, Glioblastoma multiforme, Glioma, Gliomatosis cerebri, Glomus tumor, Glucagonoma, Gonadoblastoma, Granulosa cell tumor, Hairy cell leukemia, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin's lymphoma, Hypopharyngeal cancer, Hypothalamic glioma, Inflammatory breast cancer, Intraocular melanoma, Islet cell carcinoma, Islet cell tumor, Juvenile myelomonocytic leukemia, Kaposi's sarcoma, Kidney cancer, Klatskin tumor, Krukenberg tumor, Laryngeal cancer, Lentigo maligna melanoma, Leukemia, Lip and oral cavity cancer, Liposarcoma, Lung cancer, Luteoma, Lymphangioma, Lymphangiosarcoma, Lymphoepithelioma, Lymphoid leukemia, Lymphoma, Macroglobulinemia, Malignant fibrous histiocytoma, Malignant fibrous histiocytoma of bone, Malignant glioma, Malignant mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel cell carcinoma, Mesothelioma, Metastatic squamous neck cancer with occult primary, Metastatic urothelial carcinoma, Mixed Millerian tumor, Monocytic leukemia, Mouth cancer, Mucinous tumor, Multiple endocrine neoplasia syndrome, Multiple myeloma, Mycosis fungoides, Myelodysplasia disease, Myelodysplasia syndromes, Myeloid leukemia, Myeloid sarcoma, Myeloproliferative disease, Myxoma, Nasal cavity cancer, Nasopharyngeal cancer, Nasopharyngeal carcinoma, Neoplasm, Neurinoma, Neuroblastoma, Neurofibroma, Neuroma, Nodular melanoma, Non-Hodgkin's lymphoma, Nonmelanoma skin cancer, Non-small cell lung cancer, Ocular oncology, Oligoastrocytoma, Oligodendroglioma, Oncocytoma, Optic nerve sheath meningioma, Oral cancer, Oropharyngeal cancer, Osteosarcoma, Ovarian cancer, Ovarian epithelial cancer, Ovarian germ cell tumor, Ovarian low malignant potential tumor, Paget's disease of the breast, Pancoast tumor, Pancreatic cancer, Papillary thyroid cancer, Papillomatosis, Paraganglioma, Paranasal sinus cancer, Parathyroid cancer, Penile cancer, Perivascular epithelioid cell tumor, Pharyngeal cancer, Pheochromocytoma, Pineal parenchymal tumor of intermediate differentiation, Pineoblastoma, Pituicytoma, Pituitary adenoma, Pituitary tumor, Plasma cell neoplasm, Pleuropulmonary blastoma, Polyembryoma, precursor T-lymphoblastic lymphoma, Primary central nervous system lymphoma, Primary effusion lymphoma, Primary hepatocellular cancer, Primary liver cancer, Primary peritoneal cancer, Primitive neuroectodermal tumor, Prostate cancer, Pseudomyxoma peritonei, Rectal cancer, Renal cell carcinoma, Respiratory tract carcinoma involving the NUT gene on chromosome 15, Retinoblastoma, Rhabdomyoma, Rhabdomyosarcoma, Richter's transformation, Sacrococcygeal teratoma, Salivary gland cancer, Sarcoma, Schwannomatosis, Sebaceous gland carcinoma, Secondary neoplasm, Seminoma, Serous tumor, Sertoli-Leydig cell tumor, Sex cord-stromal tumor, Sszary syndrome, Signet ring cell carcinoma, Skin cancer, Small blue round cell tumor, Small cell carcinoma, Small cell lung cancer, Small cell lymphoma, Small intestine cancer, Soft tissue sarcoma, Somatostatinoma, Soot wart, Spinal cord tumor, Spinal tumor, Splenic marginal zone lymphoma, Squamous cell carcinoma, Stomach cancer, Superficial spreading melanoma, Supratentorial primitive neuroectodermal tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat cancer, Thymic carcinoma, Thymoma, Thyroid cancer, Transitional cell cancer of renal pelvis and ureter, Transitional cell carcinoma, Urachal cancer, Urethral cancer, Urogenital neoplasm, Uterine sarcoma, Uveal melanoma, Vaginal cancer, Verner-Morrison syndrome, Verrucous carcinoma, Visual pathway glioma, Vulvar cancer, Waldenstrom macroglobulinemia, Warthin's tumor, Wilms' tumor, or any combinations thereof. In preferred embodiments, ovarian cancer can be treated, particularly HER2-positive cancerous ovarian cells.

### Materials and Methods

Materials and Instruments. Unless otherwise noted, all reagents were used without further purification. Fmoc-protected amino acids and coupling reagents were obtained from GL Biochem Ltd. (Shanghai, China). Rink amide resin were obtained from Biotage. 5(6)-TAMRA and Fluorescein 5-isothiocyanate (FITC) were purchased from Beijing Okeanos Technology Co., Ltd. (Beijing, China). Phenylsilane, Pd(PPh3)4, trifluoroacetic acid, triisopropylsilane, 4-aminophenol, 3-aminophenol, p-phenylenediamine and dibenzocyclooctyne-maleimide were purchased from J&K Scientific Ltd. (Beijing, China). 2-azidoacetic acid and DBCO-TAMRA (Catalogue number 760773) were purchased from Sigma-Aldrich Co. (USA). Native human IgG Fc (Catalogue number ab90285) and native human IgG (Catalogue number ab91102) were purchased from Abcam. Mouse antibodies, IgG1 (Catalogue number C01457M), IgG2a (Catalogue number C01693M) and IgG2b (Catalogue number C01692M), were purchased from Meridian Life Science. IgG, rabbit (Catalogue number NB100-2220) was purchased from Novus Biologicals. Atezolizumab (Catalogue number A2004) was purchased from SelleckChem. PD-L1 (His, Human) was purchased from GenScript. In-Gel Tryptic Digestion Kit and Ni-NTA agarose resin were purchased from Thermo Fisher Scientific Inc. (USA). Peptides characterization and purification were performed in RP-HPLC (Shimadzu, DGU-20A5, Japan). Peptides analysis were performed in an AutoFlex Speed LRF MALDI-TOF mass spectrometer (Bruker Daltonics, Germany). All gel images were captured by an ENDURO™ GDS Gel Documentation System (USA) or a Bio-Rad ChemiDoc Image System (USA). LC-MS/MS analysis was performed in nanoLC (nanoAdvance), MS (9.4T solariX FTICR) and column (Acclaim PepMap 100 C18).

Peptide synthesis. All Peptides were synthesized based on manual Fmoc-SPPS chemistry. Briefly, Rink Amide-ChemMatrix® resins (Biotage, Sweden) with a loading capacity of 0.5 mmol/g was first swelled by DCM/DMF (50% v/v). For each coupling procedure, 5 fold excess of protected amino acid, HBTU, HOBt, and DIEA (with a ratio of 1:1:1:2) in DMF was added to the resin for 35 minutes with shaking at RT. The deprotection reaction of Fmoc group was performed in 20% piperidine in DMF (v/v) after the resins were washed with DMF for 5 times. For capping the N-terminus amine, the resin was suspended in a DMF solution containing acetic anhydride (10 equivalent based on resin substitution) and DIEA (10 equivalents based on resin substitution), and shaken at RT for 30 minutes. Dde was readily removed by 2% hydrazine in DMF within 30 minutes. The protecting group (Allyl) of glutamic acid was removed with 0.05 equivalents of Pd(PPh3)4 and 20 equivalents of PhSiH3 in DCM for 1 h. The procedure for the coupling of 2-azidoacetic acid was performed with 5 equivalents azidoacetic acid/COMU/DMAP (1/1/2) in DMF overnight, and repeated twice. After completion of the synthesis, the resin was washed thoroughly with DCM and DMF, then with methanol and dried under vacuum. Normally, peptides were cleaved from the resin and side-chain deprotected by treatment with TFA/H₂O/TIPS (95/2.5/2.5) for 2 h at RT. Then the resin was filtered and rinsed twice with TFA. The crude peptide was obtained by precipitation with adding cold diethyl.

Peptide purification and characterization. The crude peptide was dissolved in 50% ACN: 50% H₂O containing 0.1% TFA. After being filtered through a 0.2 m filter, the peptide solution was injected to RP-HPLC (Shimadzu, DGU-20A5, Japan) equipped with a C18 column (Vydac 218TP C18 LC Column Sum, 250×4.6 mm ID). 0.1% TFA in H₂O (v/v) and 0.1% TFA in ACN (v/v) were used as the mobile phase A and B respectively. For all the analytical HPLC trials, the total flow rate was set to be 1 mL/min and the B concentration raised from 5% to 95% over 13 min following a linear gradient. For the purification of peptides in a larger scale by semi-prep HPLC columns (Vydac 218TP C18 LC Semi-Prep Column 10 um, 250×10 mm ID), the total flow rate was set to be 3 mL/min (gradient: 0-5 minutes 5% B, 5-30 minutes 5-65% B, 30-33 minutes 65-95% B, 33-36 minutes 95% B). The peptide peaks were collected, lyophilized and confirmed by MALDI-TOF mass spectrometry analysis (Bruker Daltonics, Germany).

IgG or IgG Fc labelling reactions. Unless otherwise noted, the final concentrations of proteins were 4 μM and peptides were 24 μM. The conjugation reactions were performed in PBS buffer (pH 7.4, 137 mM NaCl, 2.7 mM KCl, 10 mM Na₂HPO₄, 1.8 mM KH₂PO₄) at 37° C. for 1 h and stopped by 1% SDS. For copper-free click chemistry, the protein-peptide reaction mixture was first incubated with 100 uM DBCO-TAMRA at room temperature for 2 h and then denatured in the presence of loading dye. After resolved by SDS-PAGE, the gel was first observed in the fluorescence channel, and then stained with Coomassie Brilliant Blue dye.

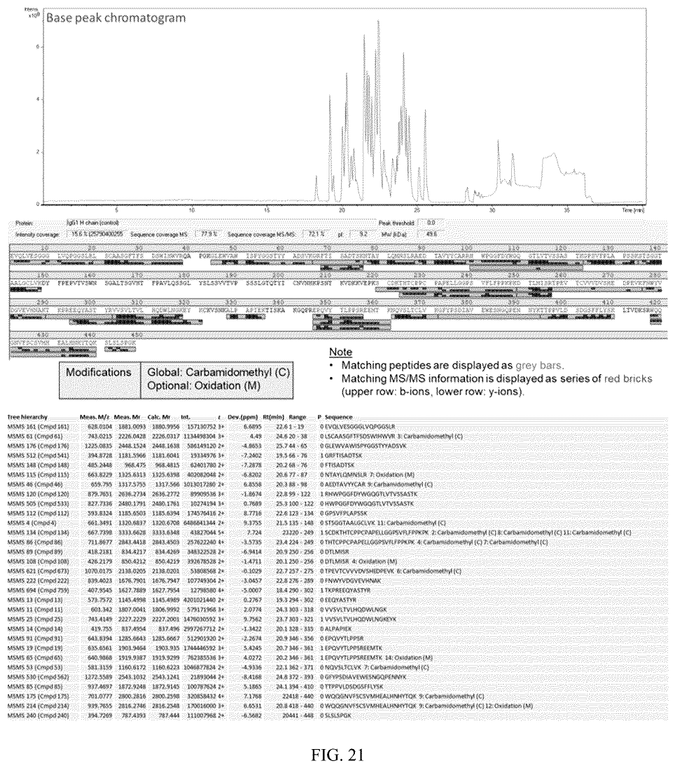

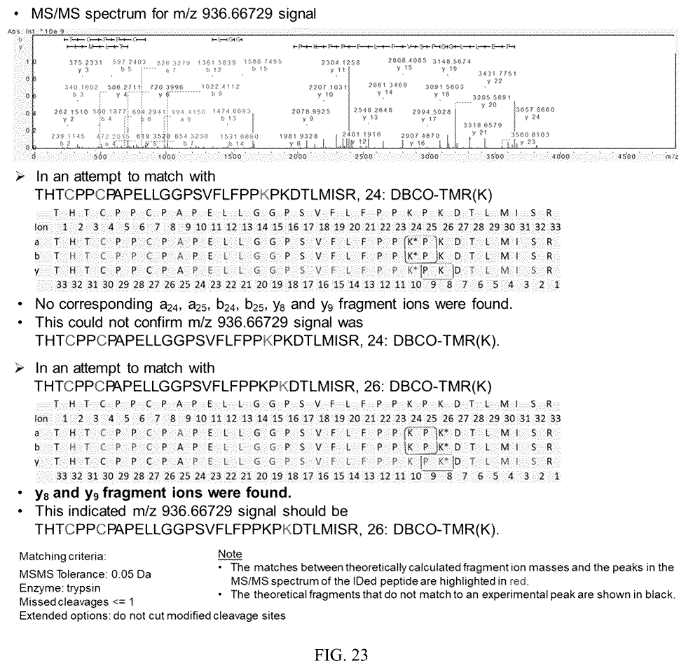

In-gel tryptic digestion for MS analysis. The loading of Fc region and antibody in SDS-PAGE gel were 2 μg and 6 μg, respectively. The band of Fe or heavy chain was cut from SDS-PAGE gel into 1×1 to 2×2 mm pieces and placed into a 600 μL receiver tube. The In-Gel Tryptic Digestion Kits were purchased from Thermo Fisher Scientific Inc. Digestion of the proteins followed the protocol provided in the kits. After the sample was digested at 37° C. for 4 h, appropriate 1% trifluoroacetic acid was added to stop this digestion reaction. The sample was ready for MS analysis. For MALDI-TOF MS analysis, the peaks were assigned, and the observed peptide fragments of the heavy chain of Atezolizumab were labelled in blue (FIG. 21). For LC-MS/MS analysis, the sequence coverage is 77.9% for MS and 72.1% for MS/MS (FIG. 23).

Characterization of IgG or IgG Fc modification by MALDI-TOF MS. To characterize Fc region, 5 μg protein sample was used. Firstly, Fc was treated in the presence of TCEP (20 mM) at 60° C. for 10 min. Then, the sample was acidified with TFA at a pH of <4. After prewetting, C4 ZipTip was equilibrated with water (0.1% TFA). Binding of Fc was realized by aspirating and dispensing sample 7 to 10 cycles. The tip was next washed 3 times and eluted by 5 μL 70% acetonitrile/water solution with 0.1% TFA. Now the sample was ready for MALDI-TOF MS analysis. For characterization of modified Fc region, the reaction was first quenched by acidification (For acid labile antibodies, the peptide f-F0 could be used to quench the reaction.). After coupling with DBCO-TAMRA, the sample was reduced. Then, the sample was desalted and concentrated by using the same procedure. To characterize the modification of atezolizumab, the modified sample (12 μg) was reduced. Next the sample was purified by HPLC with C4 column, and peaks were collected and lyophilized. The sample was dissolved in 50% acetonitrile/water solution with 0.1% TFA and subjected to MALDI-TOF MS analysis.

Microscale thermophoresis (MST) binding experiments. The interactions between Atezolizumab and its binding peptides were measured in Monolith NT.115 Capillaries. The measurements were performed in phosphate buffered saline supplemented with 0.05% Tween-20 (PBS-T). A serial dilution of Atezolizumab was prepared and the fluorescent labeled peptides were kept at a constant concentration which is in the same range or lower than the expected Kd. The measurements were performed on a NanoTemper Technologies Monolith NT.115 instrument. The settings of MST power was medium and LED power was Auto-detect.

Pull-down assay for studying the binding between PD-L1 and modified atezolizumab. Ni-NTA agarose resins were resuspended and 20 μL resins were pipetted into a 1.5 mL tube. After washing with PBS-T buffer (phosphate buffered saline supplemented with 0.1% Tween-20), His-tagged human PD-L1 (200 nM) was immobilized on Ni-NTA resins in 100 μL buffer solution (20 mM imidazole, pH 7.4 PBS-T) at room temperature for 1 h with shaking. The binding solution was removed by centrifugation and the resins were washed three times with pH 7.4 PBS-T buffer. Then Atezo-TAMRA (100 nM) was incubated with the resins in 100 μL buffer solution (20 mM imidazole, pH 7.4 PBS-T) for 1 h with shaking. Next, the resins were washed three times with PBS-T and eluted by boiling the beads for 10 min in 2× sample loading buffer. The samples were analyzed by SDS-PAGE. As a negative control, same amount of polyclonal IgG was incubated with the PD-L1 loaded resins. For confocal microscopy, His-tagged human PD-L1 was firstly labelled by FITC.

Liposome and immunoliposome preparing. Liposomes were composed of 1-palmitoyl-2-oleoyl-glycero-3-phosphocholine (POPC, Avanti), cholesterol and L-α-Phosphatidylethanolamine-N-(7-nitro-2-1,3-benzoxadiazol-4-yl) (NBD PE, Avanti) in a molar ratio of 67:30:3. All lipids were dissolved in chloroform and evaporated under reduced pressure to form lipid films on the flask wall. The lipid films were then re-suspended in PBS buffer. After extrusion of liposomes with ten cycles through a pore size (100 nm) polycarbonate filter, the liposome was incubated with lipid-Tras at 60° C. at a ratio of 50 μg antibody/mol lipid. The mixture was incubated for 30 min with slow agitation. DLS were tested for characterization of liposome and immunoliposome. Tras: transtuzumab.

Bifunctional linkers synthesis and characterization. Unless otherwise noted, the chemicals used for linkers synthesis were form Sigma Aldrich. 5-Norbonene-2-methanamine (1 mM) or methyl-tetrazine-amine was incubated with the DBCO-PEG4-NHS ester (1.2 mM) in phosphate buffer (pH 8.2) at RT for 3 h. The reaction mixture was purified by HPLC and lyophilized. The reaction mixture was diluted in 50% ACN: 50% H₂O containing 0.1% TFA. After being filtered through a 0.2 m filter, the peptide solution was injected to RP-HPLC (Shimadzu, DGU-20A5, Japan) equipped with a C18 column (Vydac 218TP C18 LC Column Sum, 250×4.6 mm ID). 0.1% TFA in H₂O (v/v) and 0.1% TFA in ACN (v/v) were used as the mobile phase A and B respectively. For all the analytical HPLC trials, the total flow rate was set to be 1 mL/min and the B concentration raised from 5% to 95% over 13 min following a linear gradient. For the purification of peptides in a larger scale by semi-prep HPLC columns (Vydac 218TP C18 LC Semi-Prep Column 10 um, 250×10 mm ID), the total flow rate was set to be 3 mL/min (gradient: 0-5 minutes 5% B, 5-30 minutes 5-65% B, 30-33 minutes 65-95% B, 33-36 minutes 95% B). The product peaks were collected, lyophilized and confirmed by QEFMS Analysis (The Thermo Scientific™ Q Exactive™ Focus).

Bispecific antibody generation reaction condition. Unless otherwise noted, the final concentrations of proteins were 20 μM and linkers were 200 μM. The conjugation reactions between azidoacetyl modified antibody and bifunctional linkers were performed in PBS buffer (pH 7.4, 137 mM NaCl, 2.7 mM KCl, 10 mM Na₂HPO₄, 1.8 mM KH₂PO₄) at 37° C. for 3 h. For inverse electron demand Diels-Alder reaction, the antibodies were mixed in PBS buffer at the final concentration 20 μM for 48 h at room temperature. After resolved by SDS-PAGE, the gel stained with Coomassie Brilliant Blue dye.

T Cell Isolation. CD3+ T cells were isolated from human PBMCs derived from healthy donors through negative selection using the EasySep™ Human T Cell Isolation Kit (StemCell Technologies) according to manufacturer's protocol. Flow cytometry was performed for isolated CD3+ T cells using direct monoclonal conjugate anti-human CD45 (APC anti-human CD45, IgG1 k, Clone HI30) and anti-human CD3 (FITC anti-human CD3, IgG1 k, Clone UCHT1). Cell purity was validated to be >98%. Isolated T cells were maintained in complete RPMI medium supplemented with 10% fetal bovine serum and penicillin/streptomycin (Gibco™) and incubated at 37° C. in a humidified CO₂ condition.

In-Vitro Cytotoxicity Assay. Target cell cytotoxicity was evaluated as previously described. Adherent tumor cells (SKOV3, MDAMB231) were seeded at 2.5×10⁴ cells/well in 96-well flat-bottom plates in complete RPMI medium and incubated overnight at 37° C. in a humidified 5% CO₂ atmosphere. Target cells were preincubated with either anti-CD3/HER2 bispecific antibody, anti-CD3 monoclonal antibody, or anti-HER2 monoclonal antibody for 60 minutes at 37° C., 5% CO2, prior to the addition of purified T cells in a 2:1 Effector cells/Target cells (E/T) ratio (1×10⁵ cells/well) and incubated for 48 hours. Cellular cytotoxicity was measured via the release of lactate dehydrogenase (LDH) from dead target cells by using CyQUANT™ LDH Cytotoxicity Assay (Invitrogen™) according to the manufacturer's instructions. Spontaneous LDH release was assessed using target and effector cells without antibodies. Maximal target cell lysis was achieved by incubation of target cells with lysis buffer. The percentage of cytotoxicity towards target cells was calculated based on the following formula:

% Cytotoxicity=Experiment Value−Effector Cells Spontaneous Control−Target Cells Spontaneous Control/Target Cell Maximum Control−Target Cells Spontaneous Control×100

EM studies. Protein was diluted to a final concentration of 0.01 mg/mL in PBS and applied to a glow-discharged carbon-coated 400-mesh copper TEM grid. The specimen was negatively stained with 2% (w/v) uranyl acetate. In total, 136 micrographs were recorded under low dose conditions on a FEI Talos F200C transmission electron microscope (ThermoFisher Scientific) operated at 200 kV. Data was recorded with a 4 k×4 k Ceta 16 M camera (ThermoFisher Scientific) with a pixel size of 3.22 Å per pixel at a calibrated magnification of 45,000×. A defocus range of −0.7 to 1.0 m was used. Single particles were selected automatically and processed with RELION 3.1.3 [45]. A total of 235,867 particles were extracted with box size of 256 pixels and imported into cryoSPARC 3.3.1 [46] for unbiased, reference-free 2D classifications. After removing junk particles, 114,841 and 11,616 particles were classified as monomeric and dimeric forms of bsAbC, respectively. The dataset was acquired in a single imaging session.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLES

### Example 1-Acetylation of IgG Fc Domain

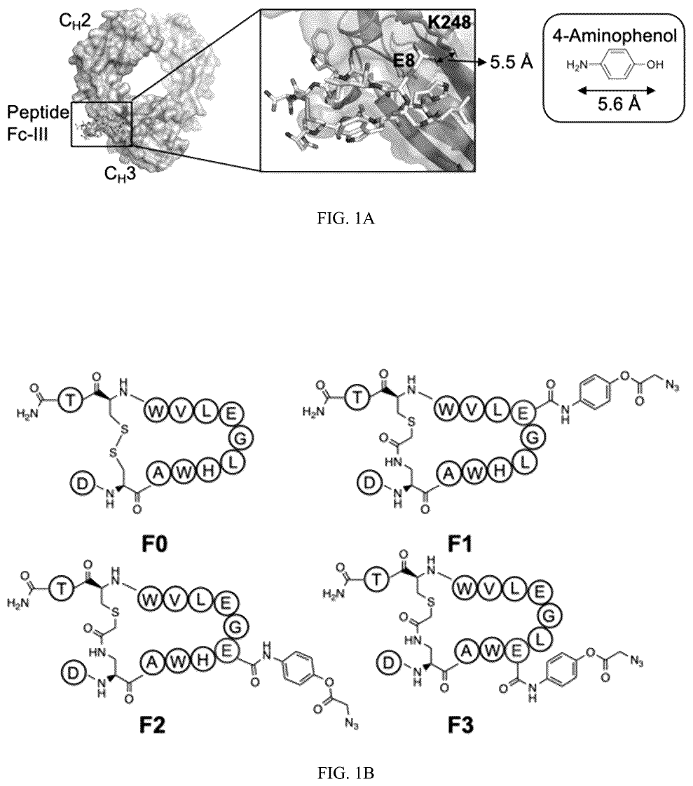

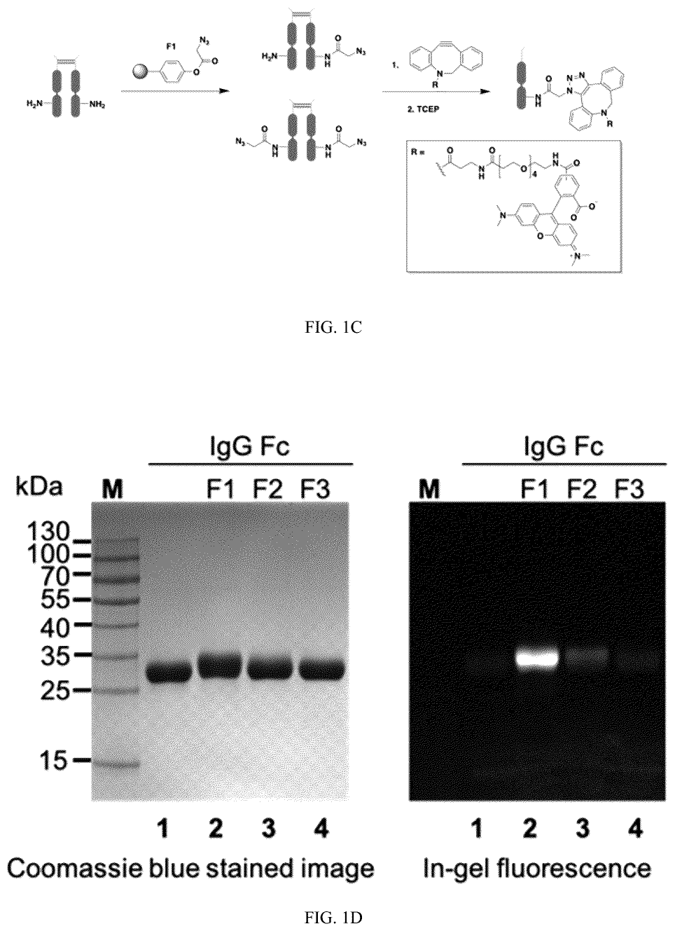

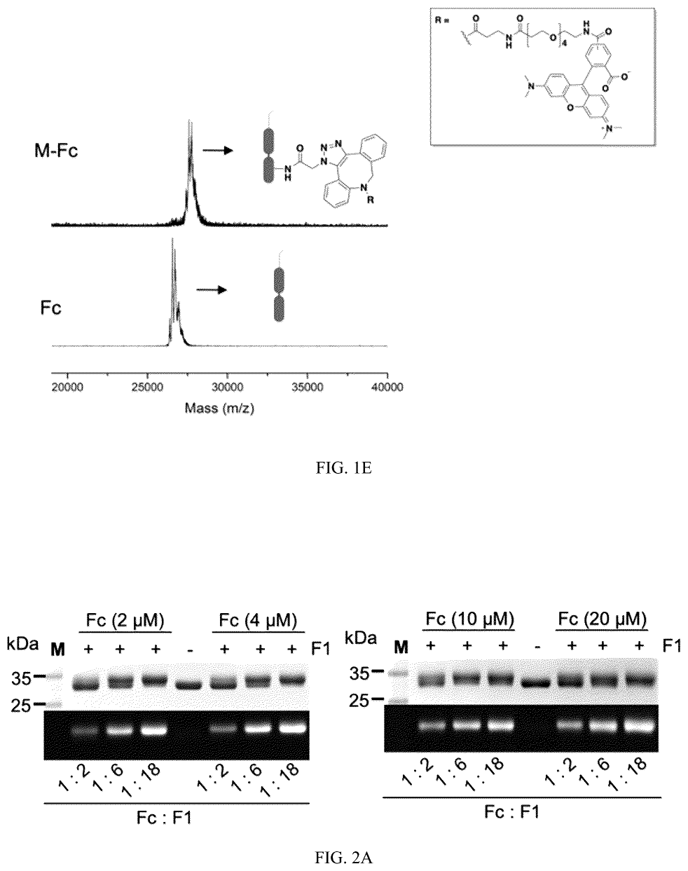

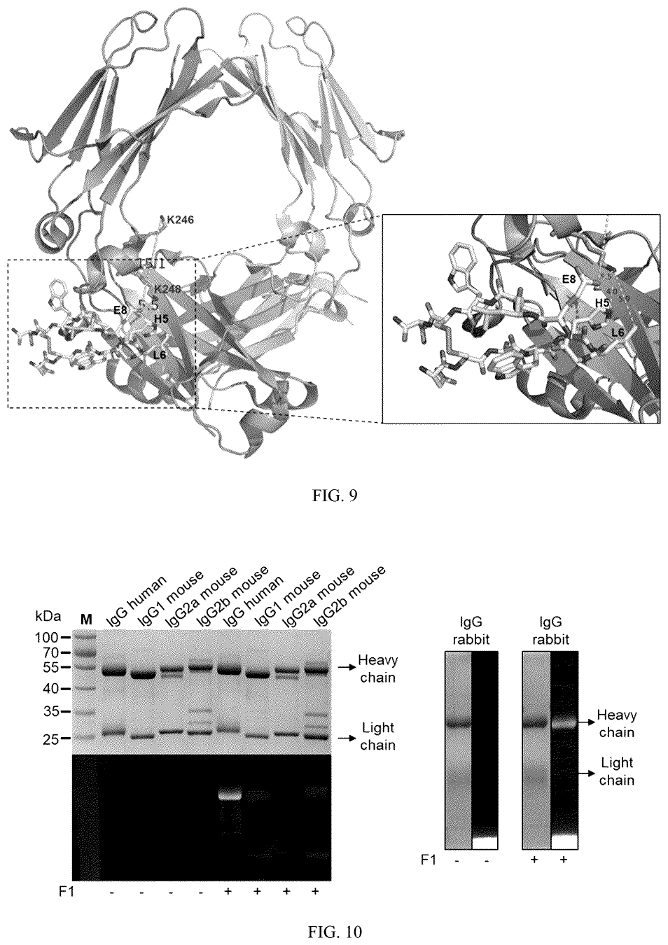

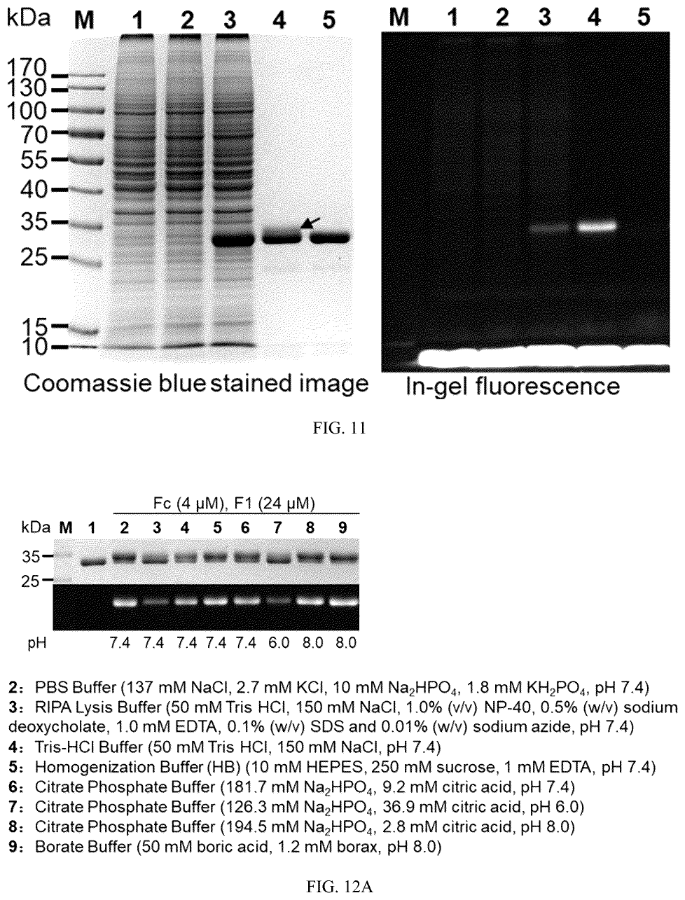

The Fc-III peptide identified from bacteriophage libraries specifically binds the hinge region of the IgG Fc with a high affinity (Kd˜20 nM) [19]. According to the co-crystal structure of the Fc-III peptide and IgG Fc (PDB ID: 1DN2) (FIG. 1A), His5, Lys6, and Glu8 of Fc-III peptide are close to Lys248 of IgG Fc with distances ranging from 4 Å to 6 Å (FIG. 9). We, therefore, chose to substitute His5, Lys6, or Glu8 with a glutamine derivative that contains a phenyl azidoacetate motif at the side chain [20-22], and synthesized peptides F1 to F3 based on the structure of Fc-III (we replaced the disulfide bond in Fc-III by a thioether bond to increase the stability, and this peptide is called F0) (FIG. 1B). When the peptide was mixed with a human IgG Fc fragment in PBS at 37° C. for 1 h, an acetylation reaction spontaneously occurred which transferred the azidoacetyl group from F1 to the IgG Fc, as indicated by the in-gel fluorescence image and MALDI-TOF MS results (FIGS. 1C-1D). Notably, the human IgG Fc used here is a mixture of glycosylated Fc proteins of the four subclasses, IgG1 to IgG4 [23]. We also found that F1 only reacted with human and rabbit IgG Fcs, but not the IgG Fc from mice (FIG. 10), which is because the Fc-III peptide we use was initially selected to bind with human and rabbit IgGs [24]. F1 could also selectively acetylate IgG Fc in a protein mixture of HeLa cell lysate without an appreciable level of background reactions (FIG. 11). This result shows that peptide binding drives the acetylation reaction of IgG Fc by a phenyl ester, and the precise positioning of the phenyl ester is the key to the reaction.

### Example 2—Kinetics and Selectivity of the Acetylation Reaction

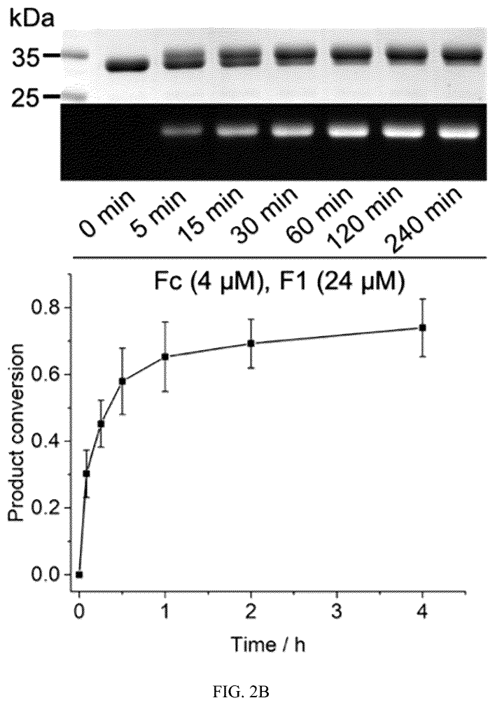

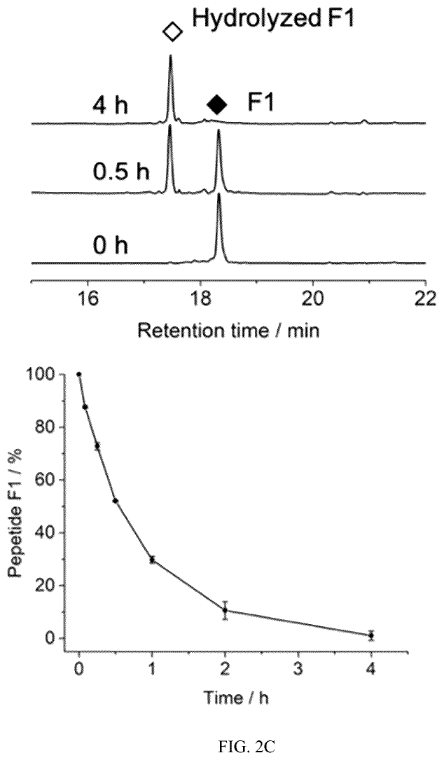

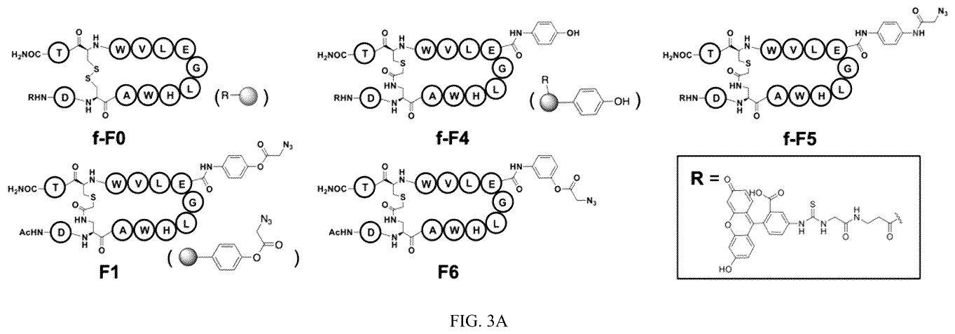

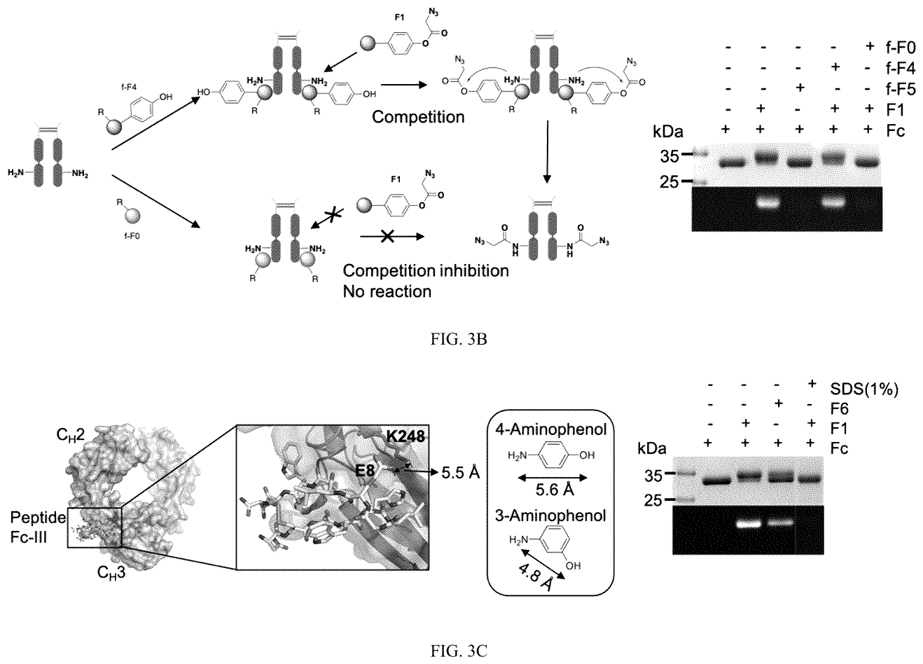

The acetylation reaction was further studied at different concentrations of the reactants. At IgG Fc concentration ranging from 2 μM to 20 μM (while keeping F1: IgG Fc ratio of 6:1), the reactions all proceeded to >50% within 1 h (FIG. 2A). Increasing the F1: IgG Fc ratio helped to push the reaction close to completion (FIG. 2A). The reaction also tolerated the presence of different buffers, salts, and even free amines, but was sensitive to the detergent SDS (FIG. 3C). The reaction rate significantly dropped at acidic pH (FIGS. 12A-12B). At 37° C. in PBS buffer of pH 7.4, the acetylation reaction reached 50% completion in less than 30 min and quickly plateaued (FIG. 2B). In the absence of IgGs, the peptide ester rapidly hydrolyzed to the non-reactive product, losing about 70% in 1 h at pH 7.4 (FIG. 2C). The rapid breakdown of the phenyl ester will be beneficial for the high selectivity of the acetylation reaction: Rapid hydrolysis of phenyl ester decreases the chance of non-selective acetylation reactions. Adding f-F0 to the reaction of F1 and IgG also abolished the acetylation reaction, suggesting that F0 competes with F1 for the same binding site on Fc (FIGS. 3A-3B). The moderate affinity can ensure a precise recognition of the binding site on the target even in a cell lysate, and at the same time accommodates sufficient structural flexibility that allows a nucleophilic reaction to occur at the interface between IgG and F1. Moreover, changing the 4-aminophenol (peptide F1) to 3-aminophenol (peptide F6) markedly decreased the acetylation yield (FIG. 3C), showing the importance of spatial organization of the electrophile.

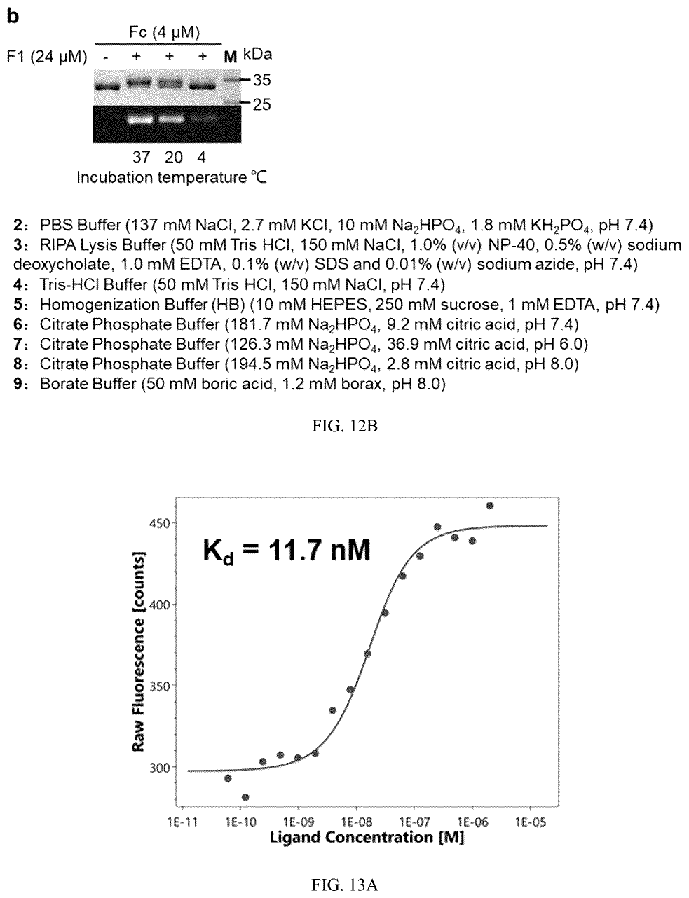

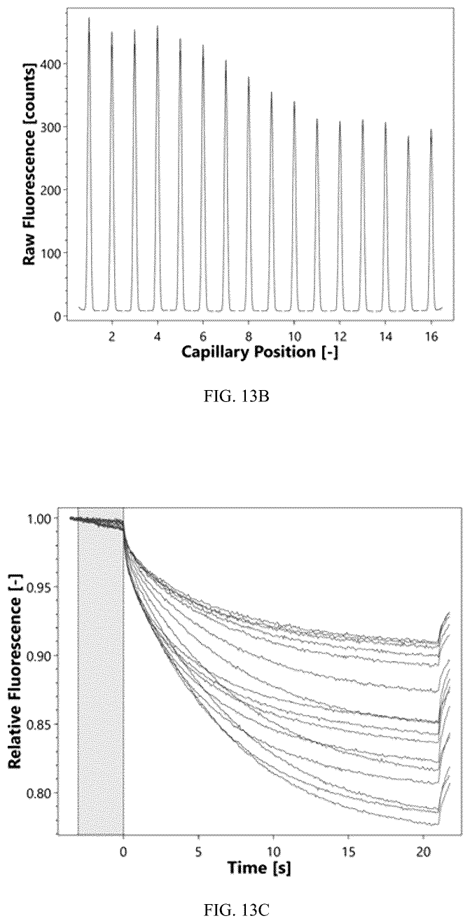

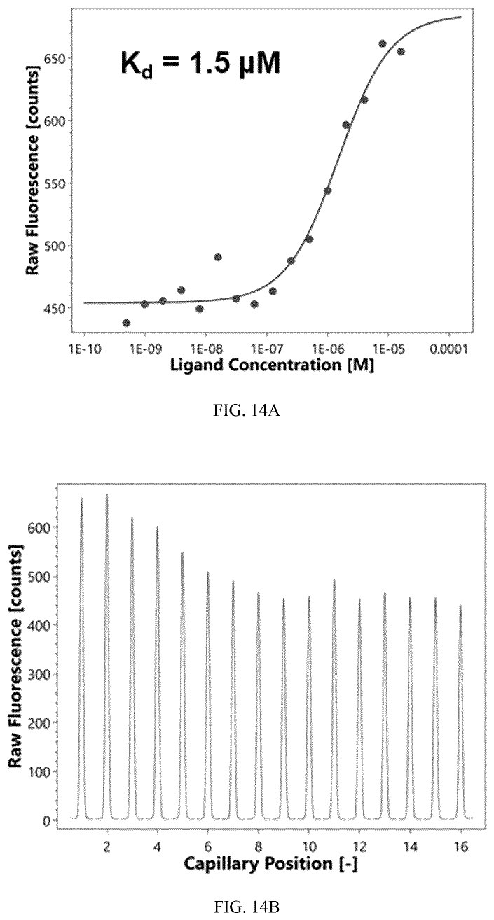

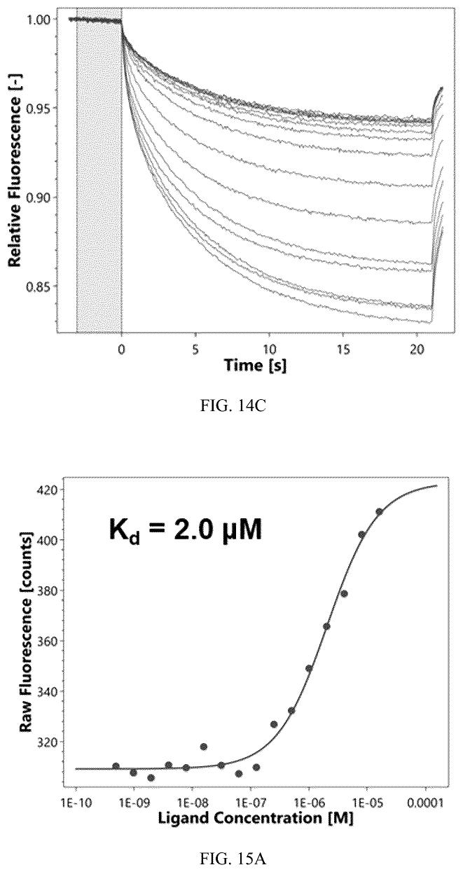

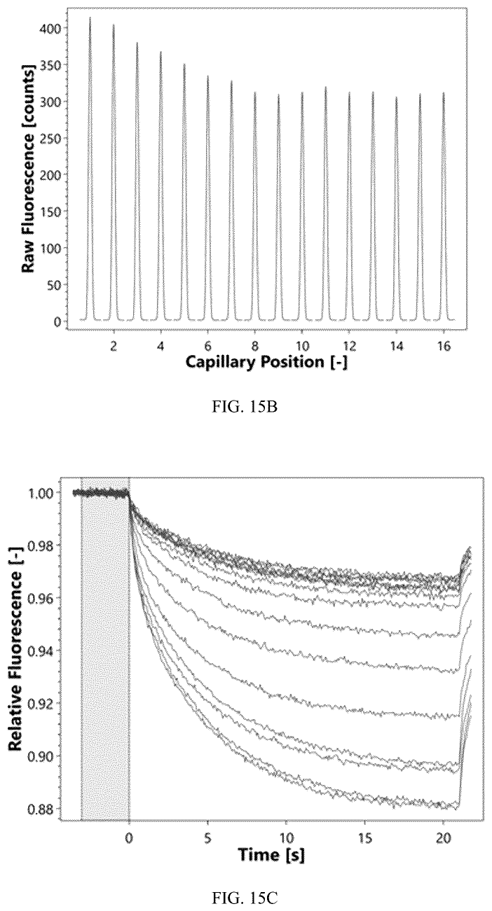

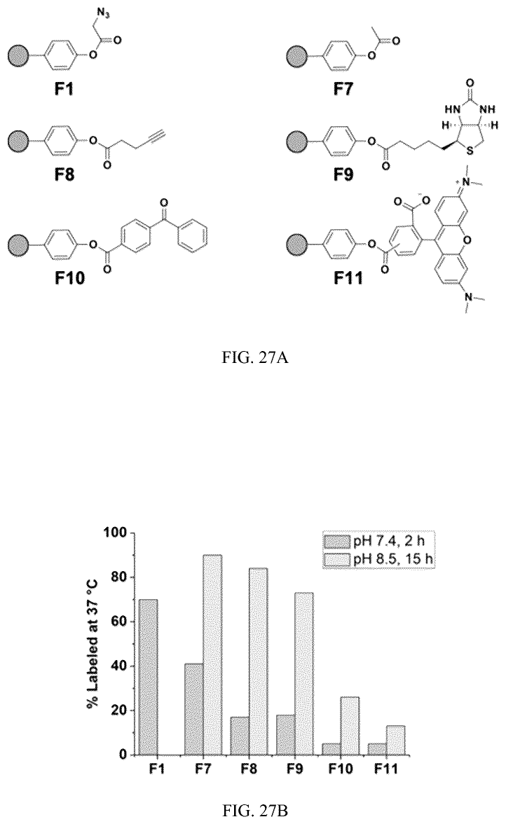

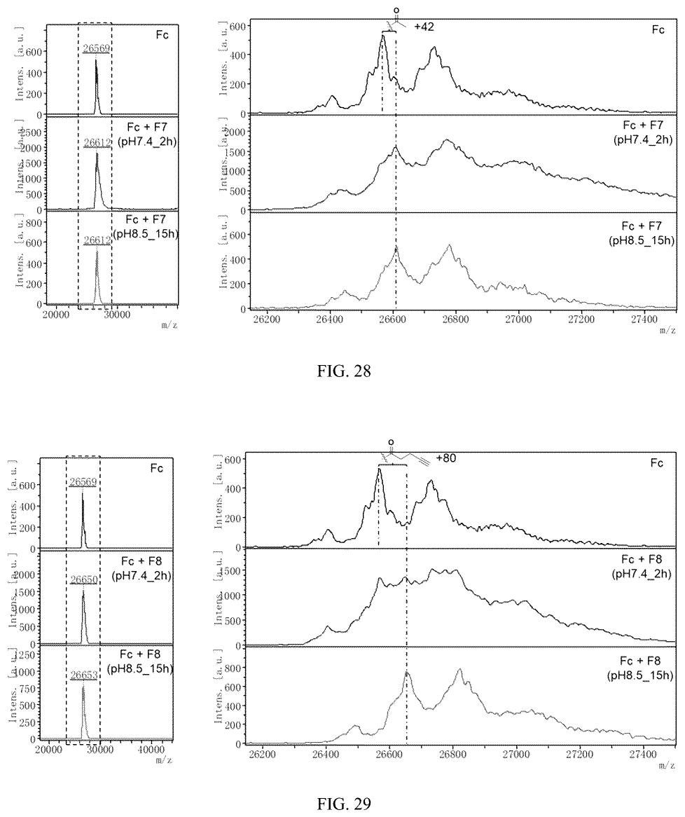

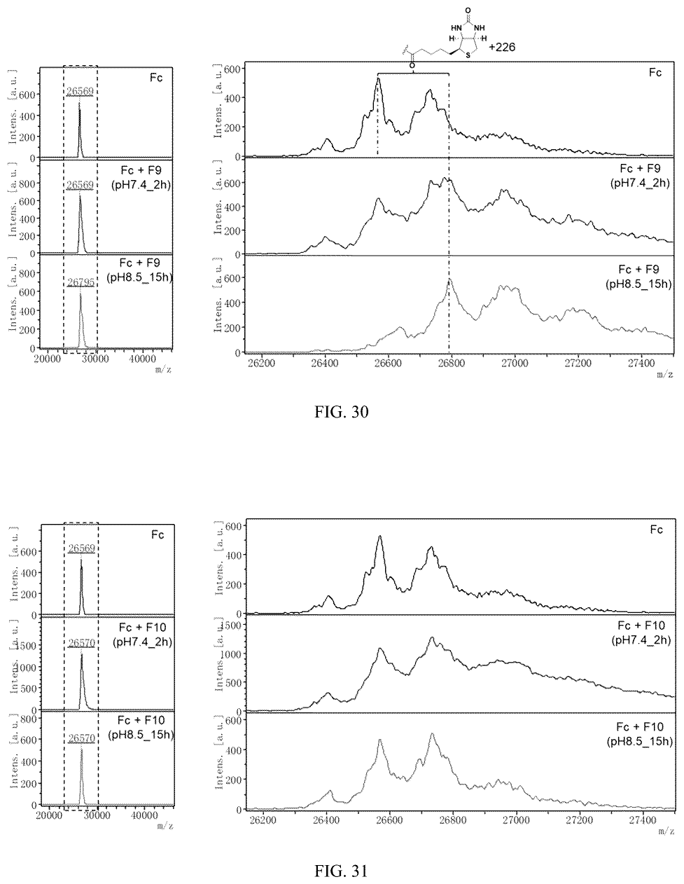

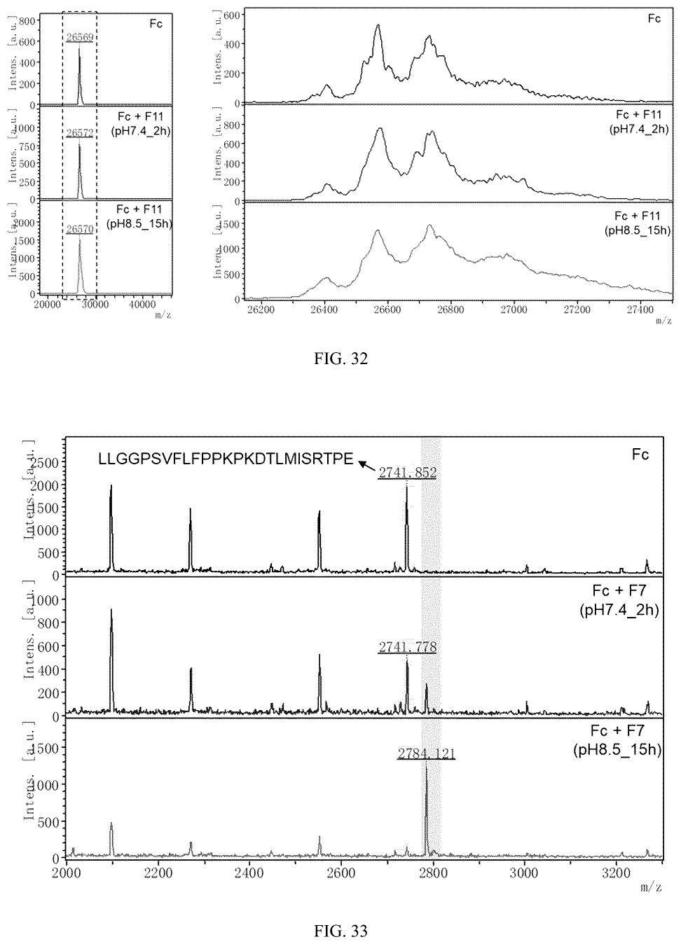

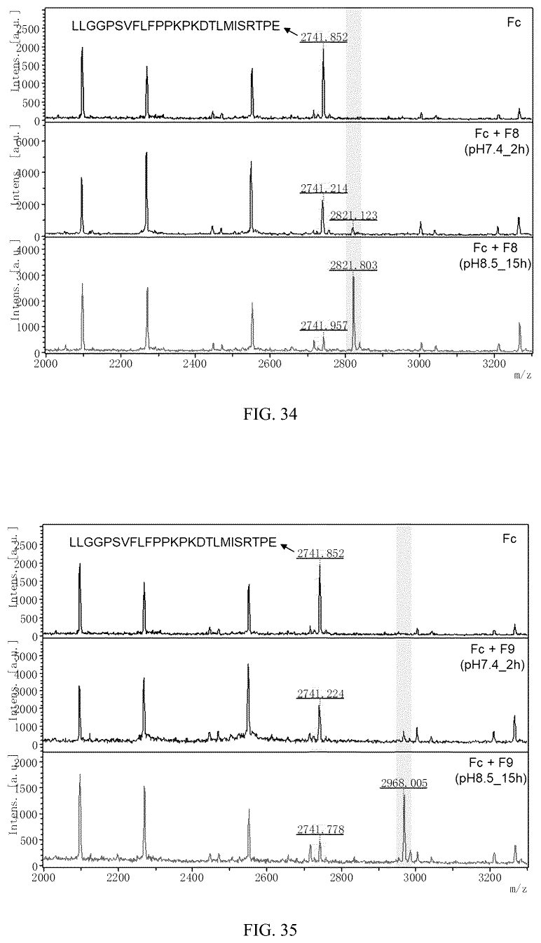

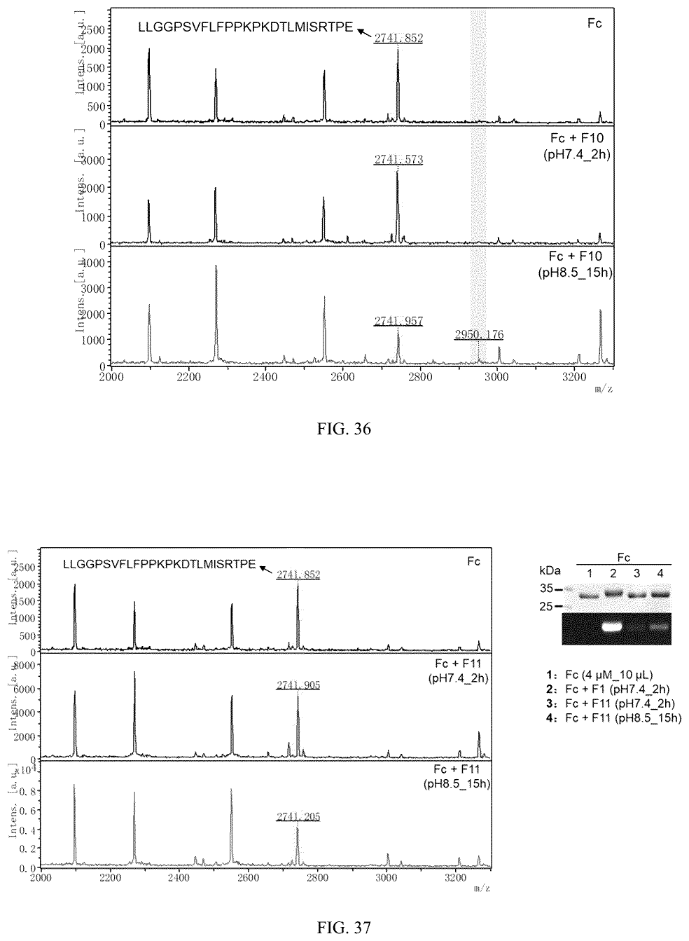

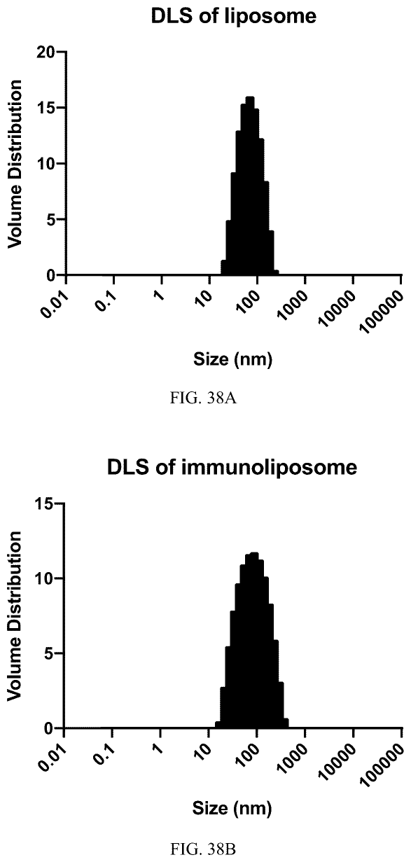

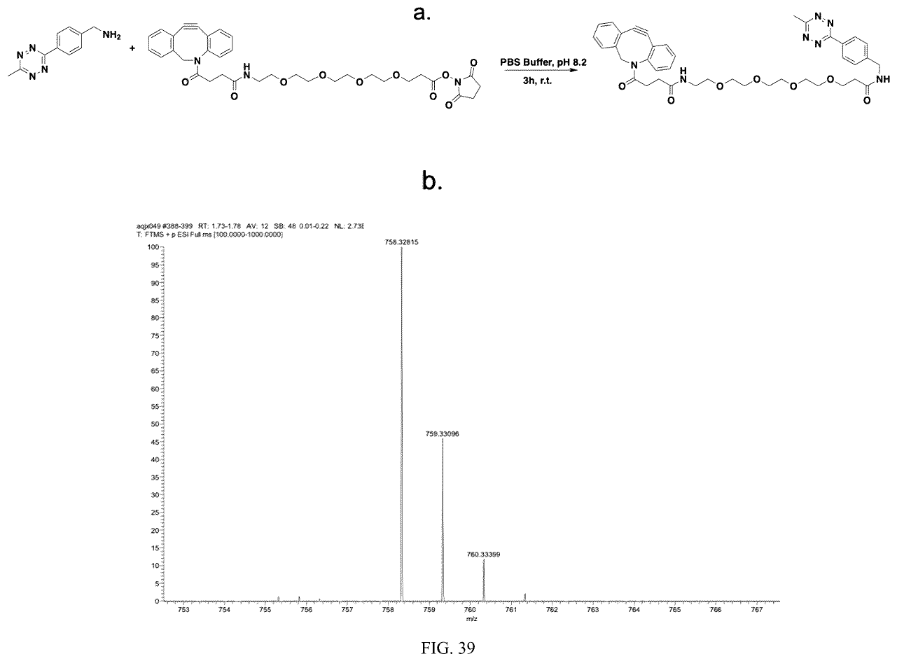

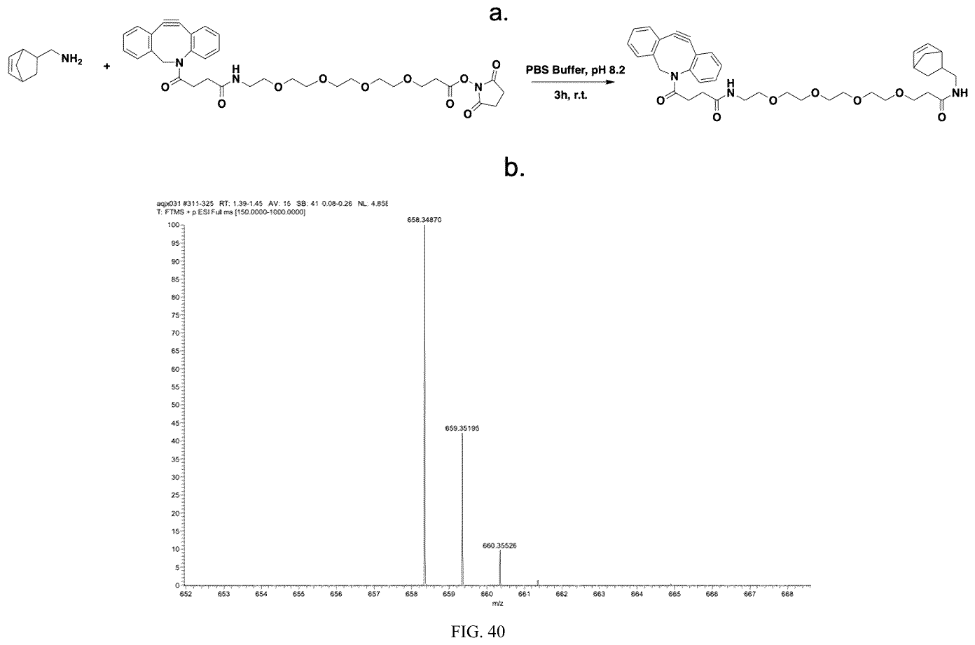

To further understand how the reaction proceeded through the bind-then-react mechanism, we measured the binding affinities of the peptide variants by microscale thermophoresis (MST). The parental Fc-III peptide (f-F0), an F1 analog in which the ester group was changed to an unreactive amide group (f-F5), and the hydrolytic product (f-F4) were synthesized, all fluorescently labeled at the N-termini (FIGS. 13A-13C). Compared with f-F0 (Kd of 11.7 nM), both f-F4 and f-F5 showed decreased binding affinities (Kd measured to be 1.5 μM and 2.0 μM respectively), and the competition experiments between reactive peptide (F1) and non-reactive peptide (f-F4 or f-F0) were shown on FIGS. 14A-14C, 15A-15C. Besides, we also synthesized several peptides with different acetyl groups to test the labeling efficiency (FIG. 27A-27B, 28-37).

### Example 3—Acetylation of Therapeutic mAbs

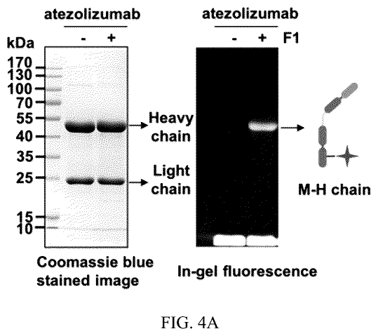

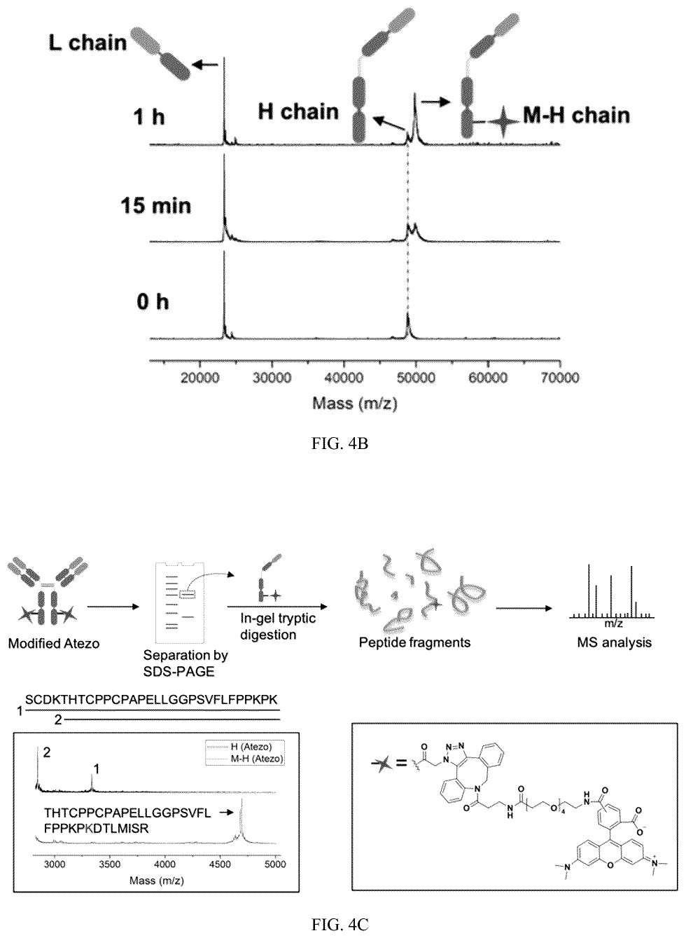

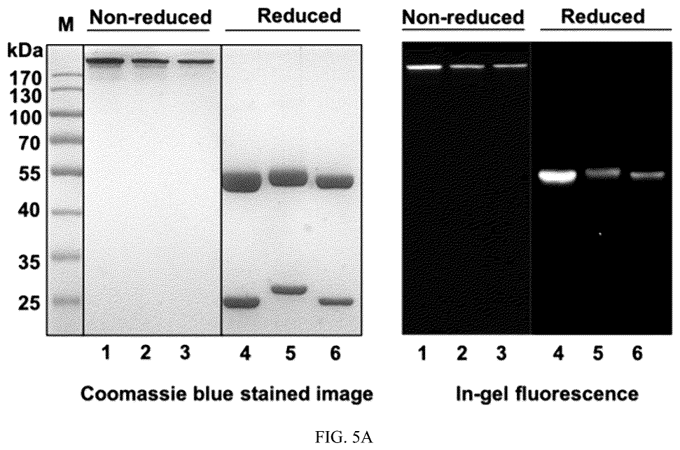

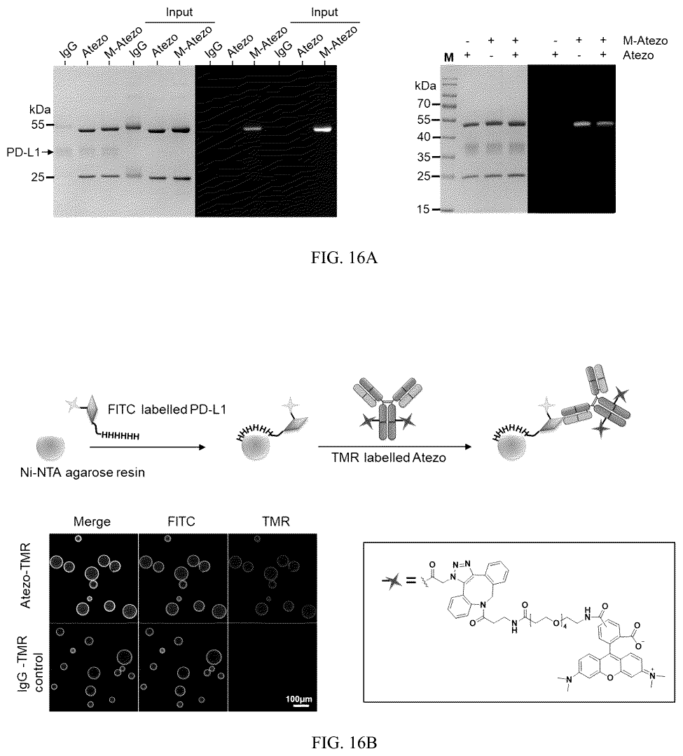

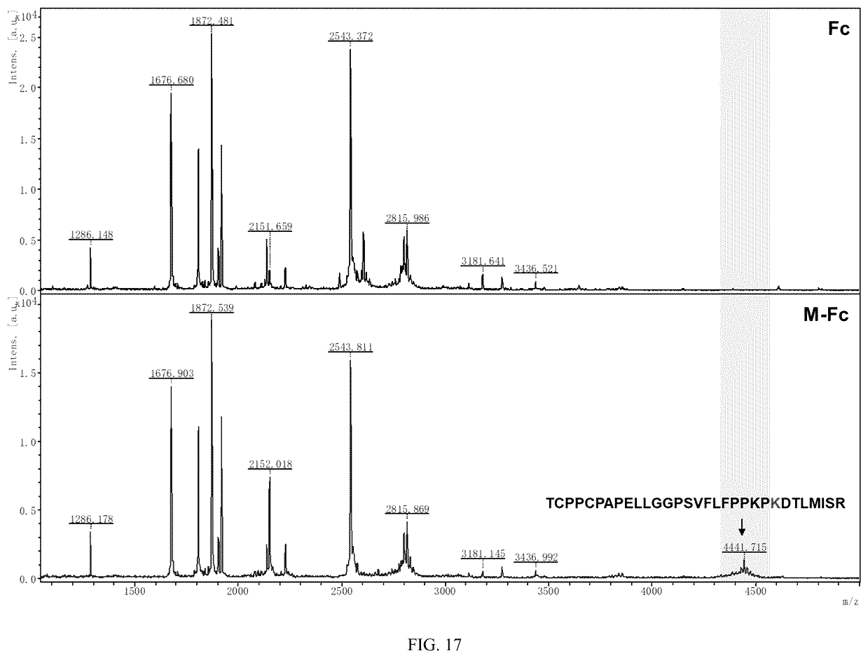

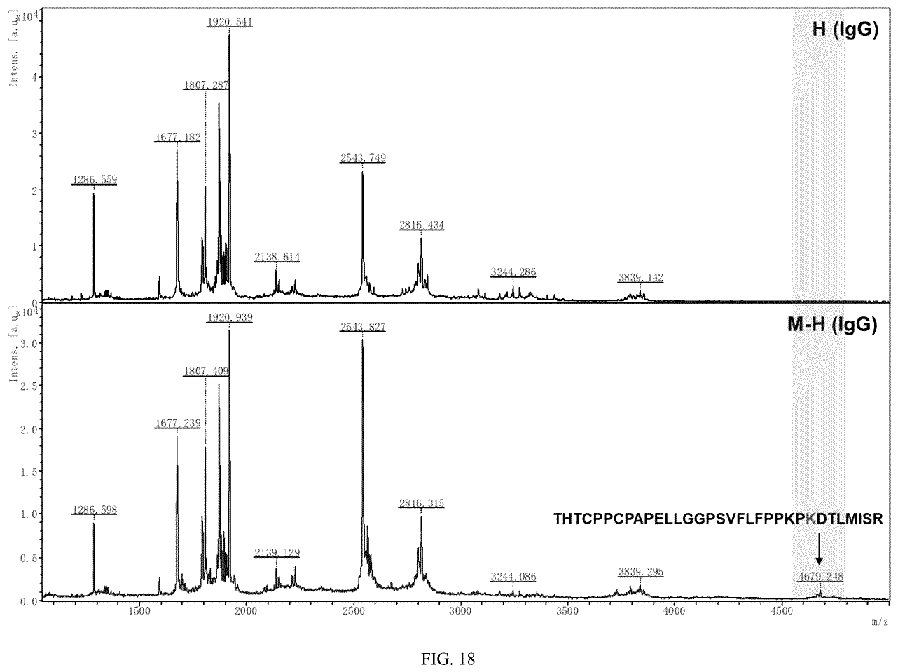

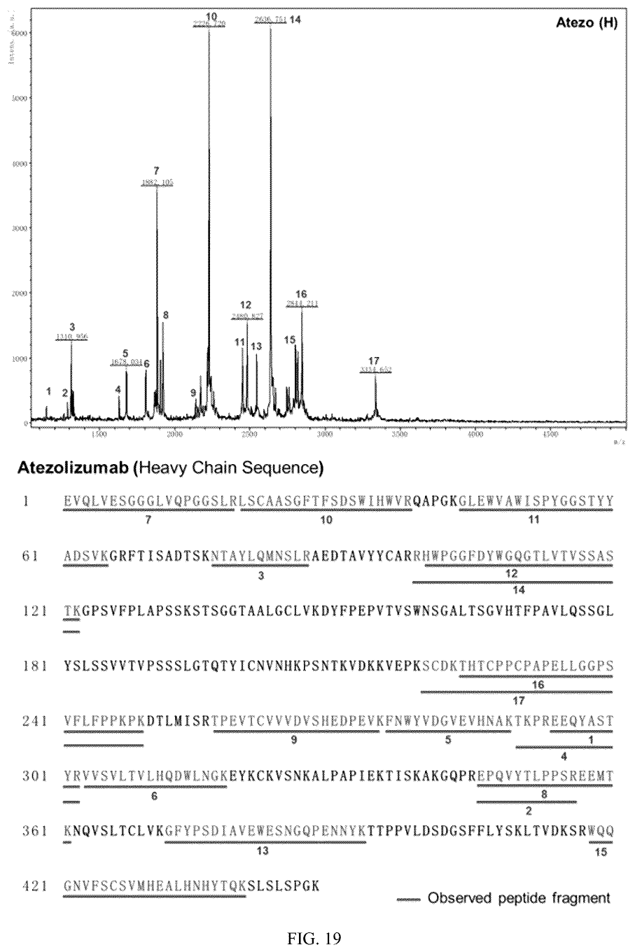

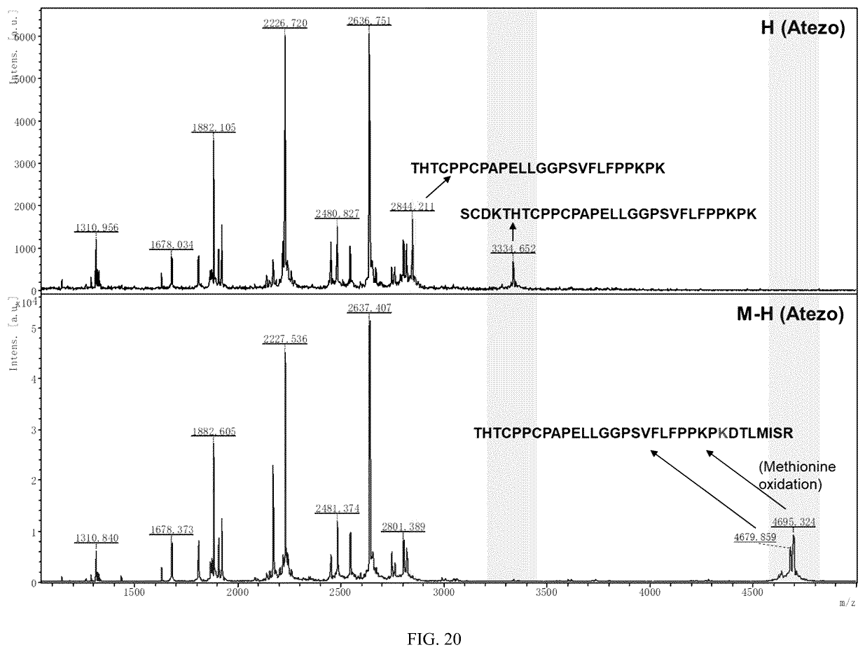

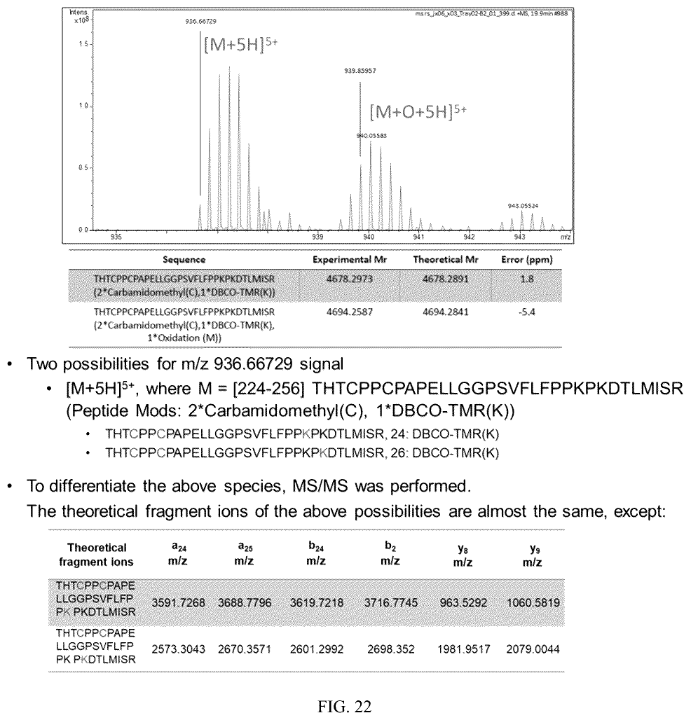

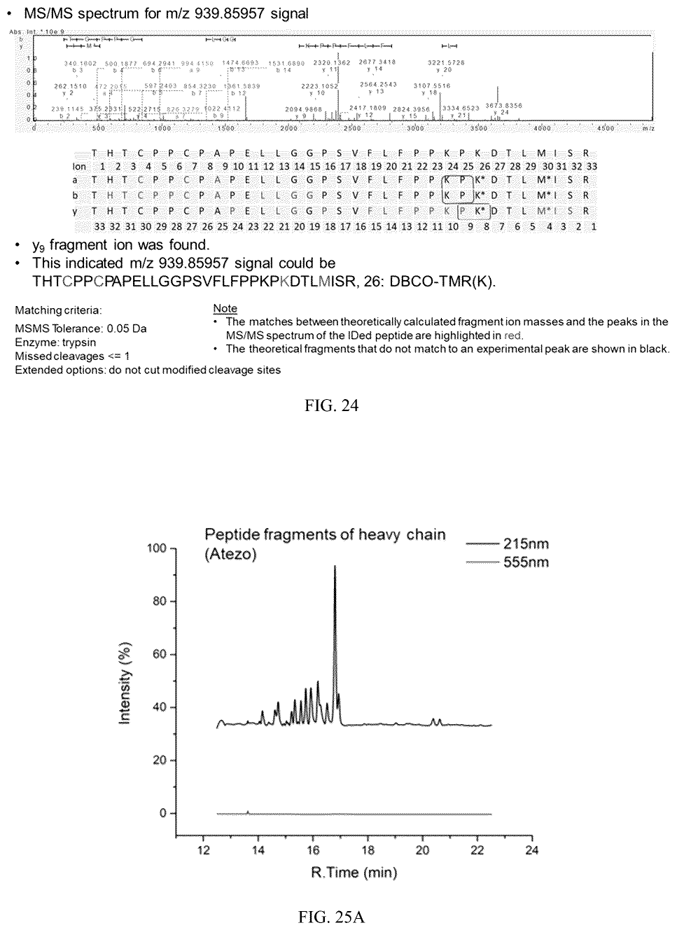

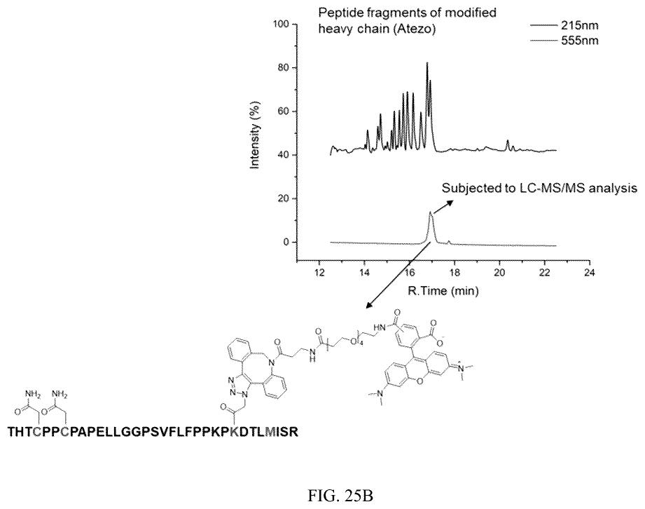

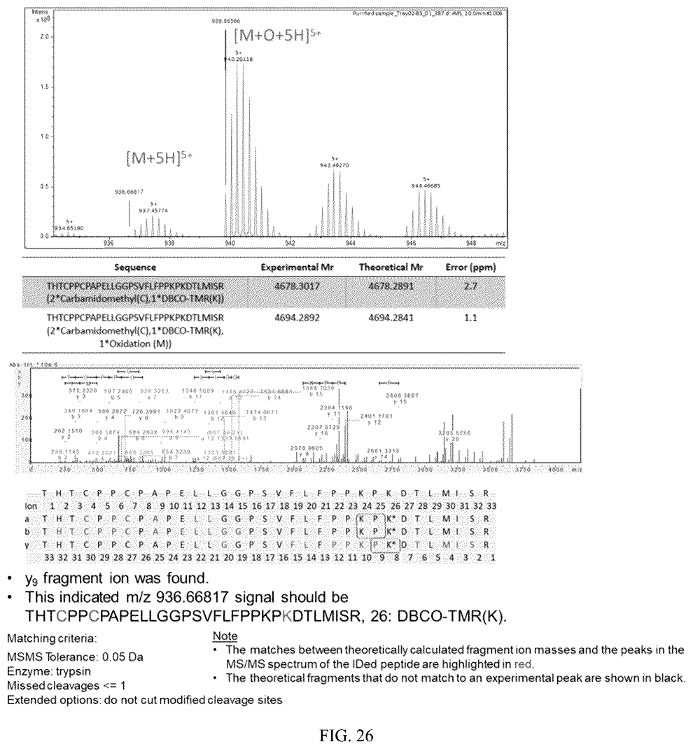

We next applied the acetylation reaction to atezolizumab, a humanized IgG1 mAb approved by the FDA in 2016 for the treatment of non-small cell lung cancer treatment by blocking the interaction of PD-L1 with both PD-1 and B7.1 [25, 26]. Incubating peptide F1 with atezolizumab at 37° C. for 1 h acetylated the heavy chain only (FIG. 4A). MALDI-TOF MS analysis revealed that estimably 50% of the heavy chain was modified in 15 minutes, and 80% in 1 h (assuming the modified heavy chain has a similar ionization property as the unmodified) (FIG. 4B). MALDI-TOF MS was analyzed to confirm the acetylation site is Lys248 in the Fc region by in-gel tryptic digestion (FIG. 4C). The wide-range MS spectra are shown in FIG. 18-26, 27A-27B. We next showed that the acetylated atezolizumab could still bind its ligand (FIGS. 16A-16B), consistent with the notion that modification of the Fc region of antibodies would not affect the Fab region [15-17]. Then we applied the acetylation to different types of therapeutic antibodies approved by the FDA (anti-HER2: trastuzumab, anti-EGFR: cetuximab, anti-CD8: daratumumab). The same results with the atezolizumab were shown by SDS-PAGE in-gel fluorescence, although to different degrees (FIG. 5A). Immunofluorescent labeling showed the acetylated atezolizumab maintained their binding with PD-L1 positive (PC12) cells, and acetylated trastuzumab bound with HER2-positive (SK-OV-3) cells respectively under confocal microscopy (FIG. 5B).

### Example 4—Antibody Lipidation to Construct Immunoliposomes

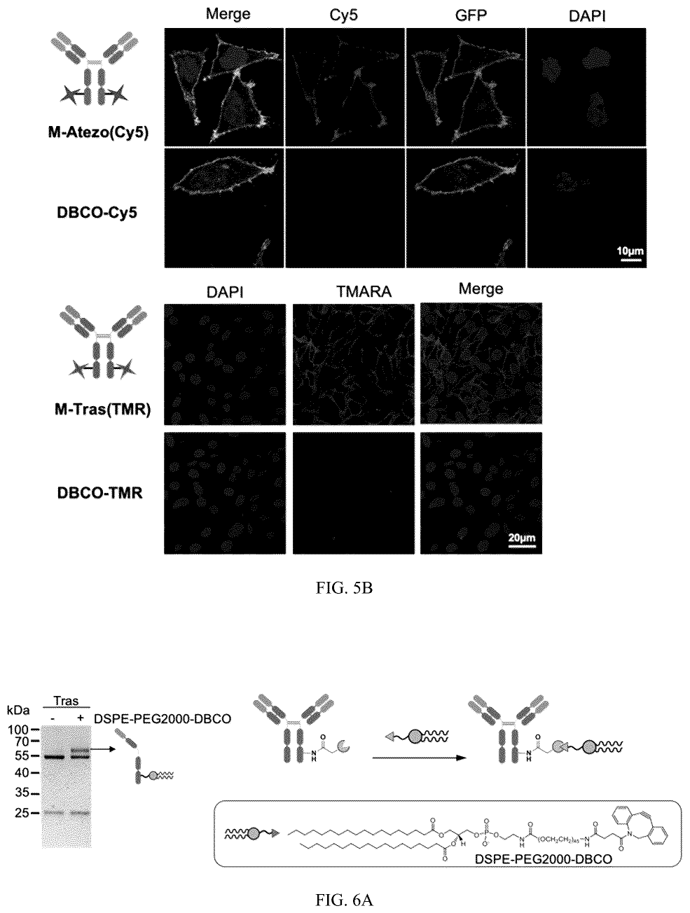

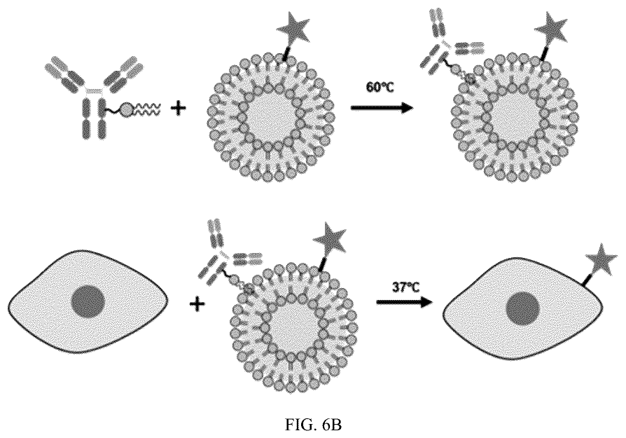

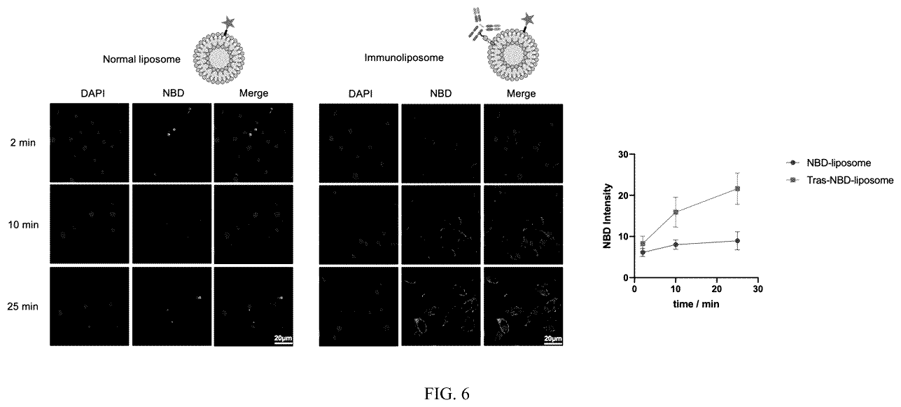

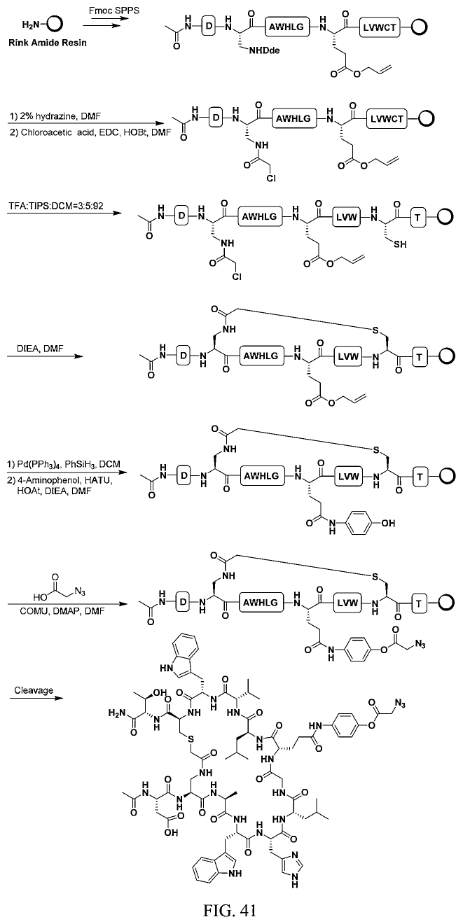

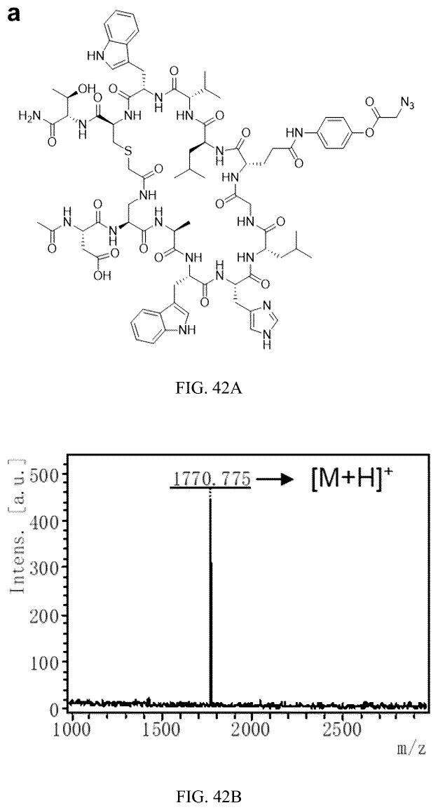

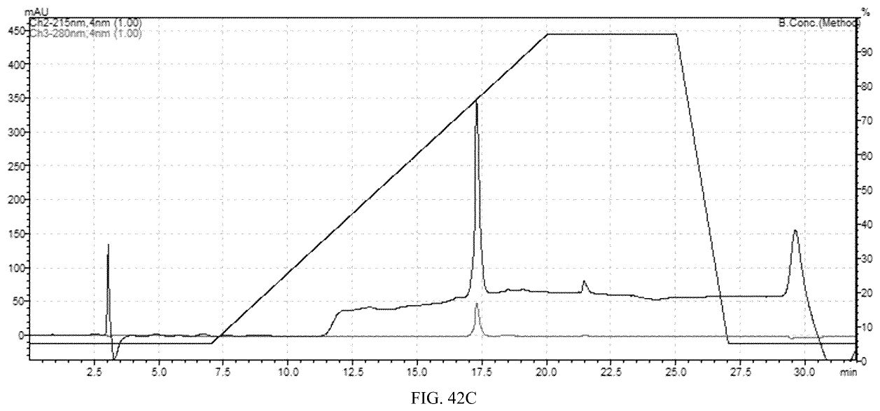

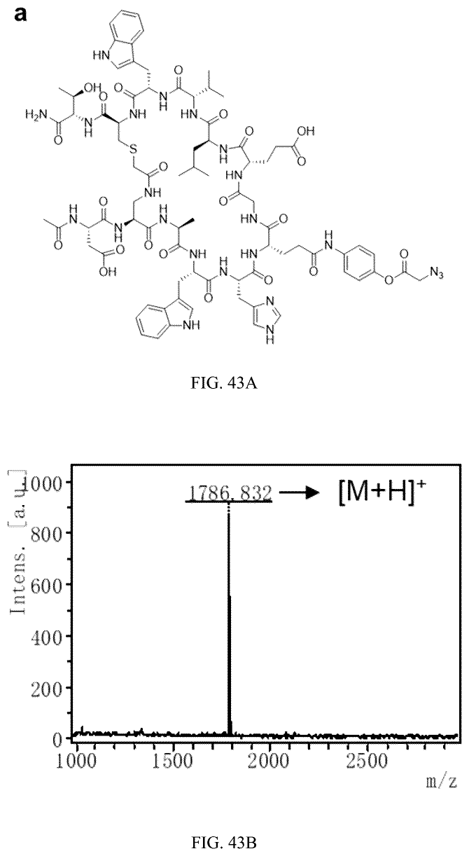

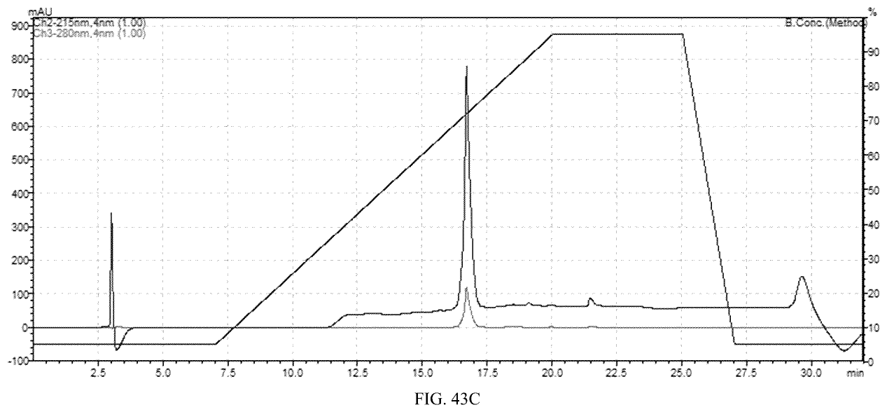

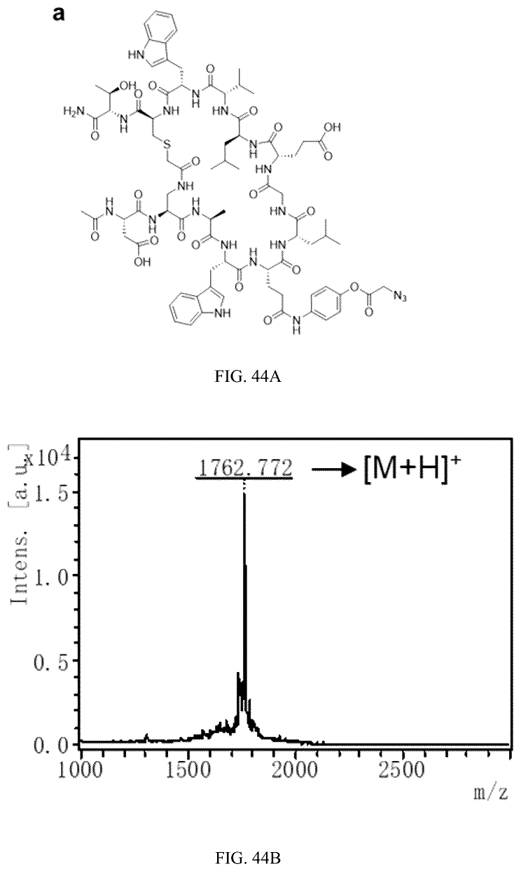

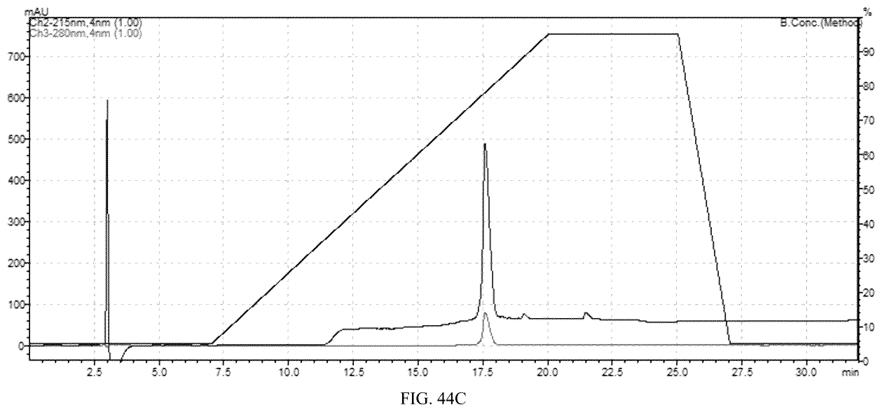

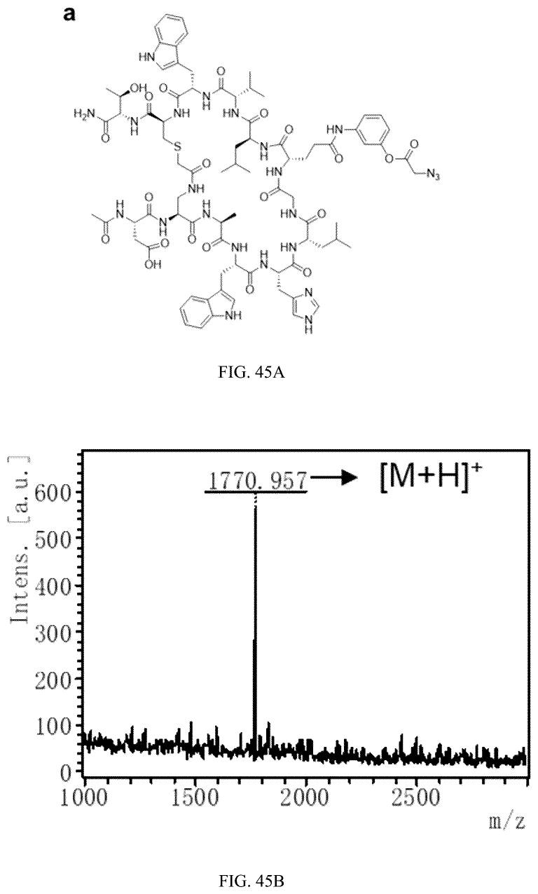

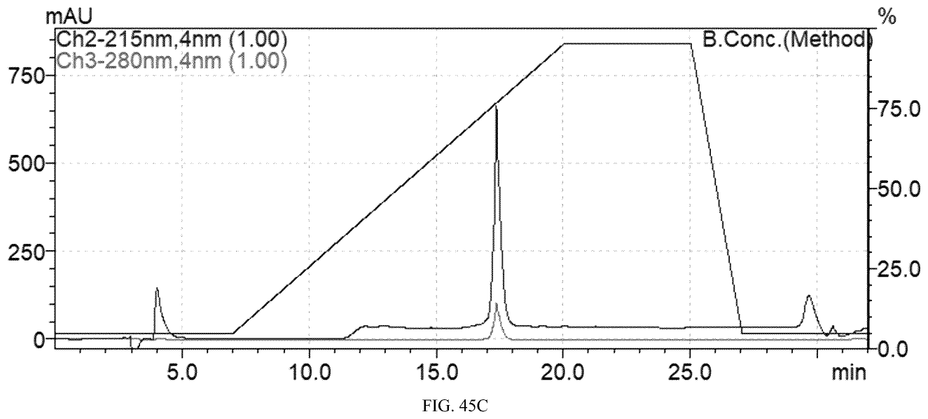

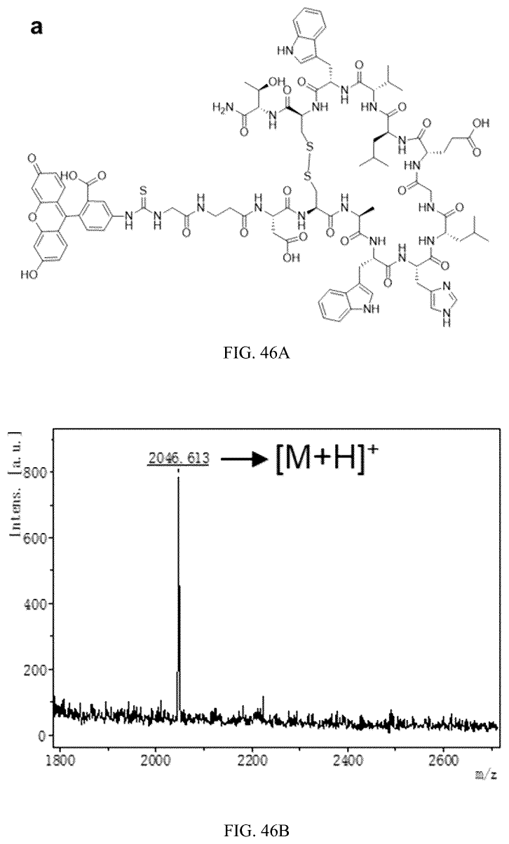

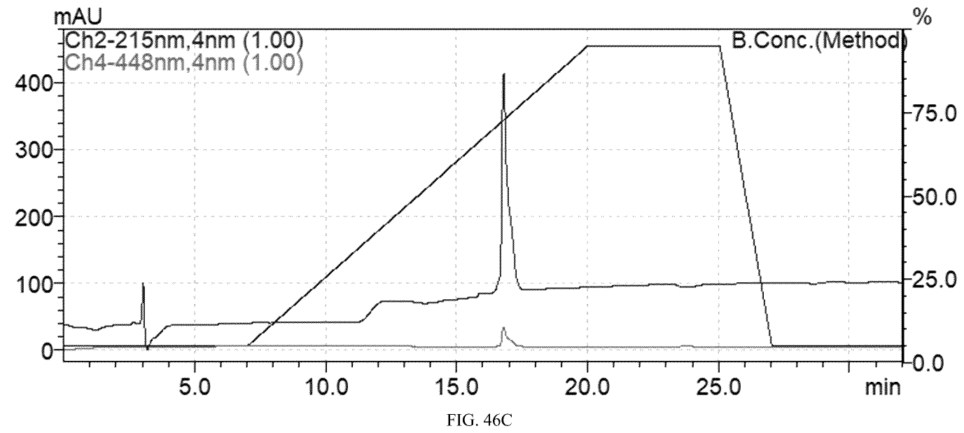

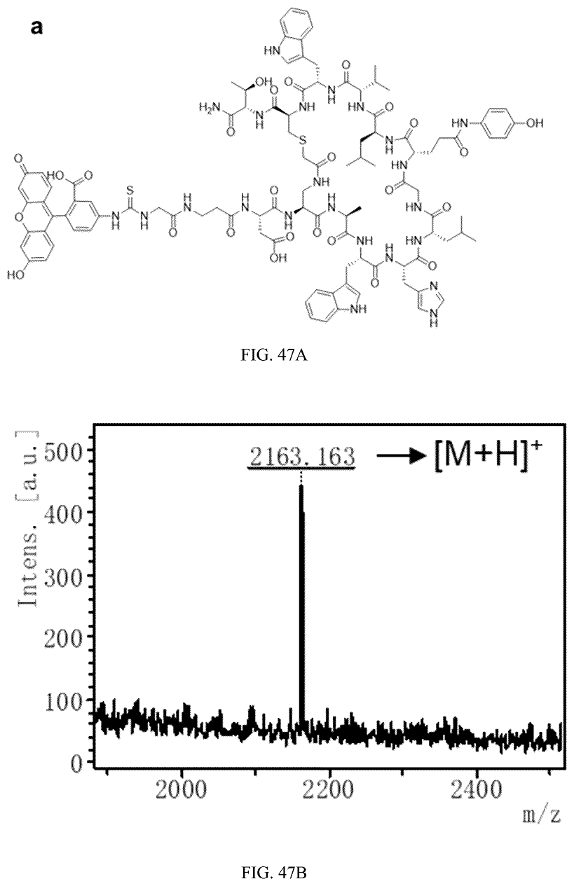

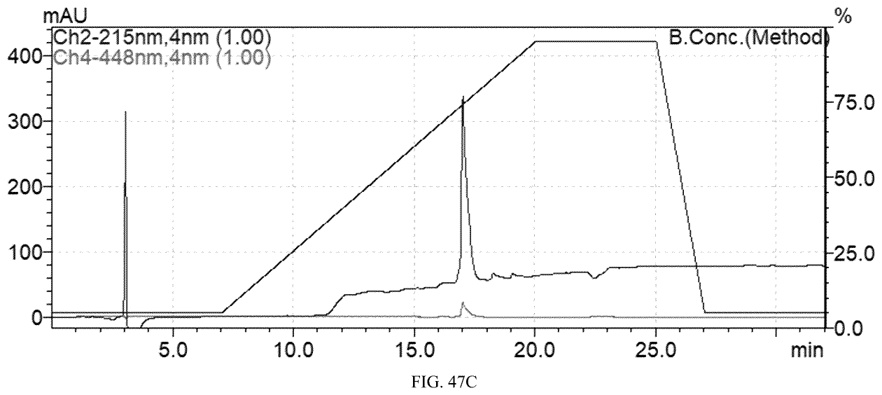

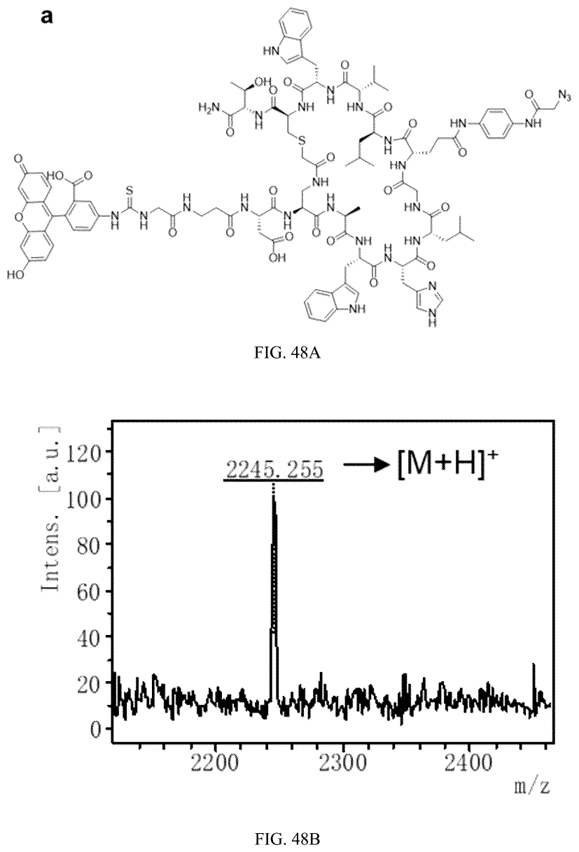

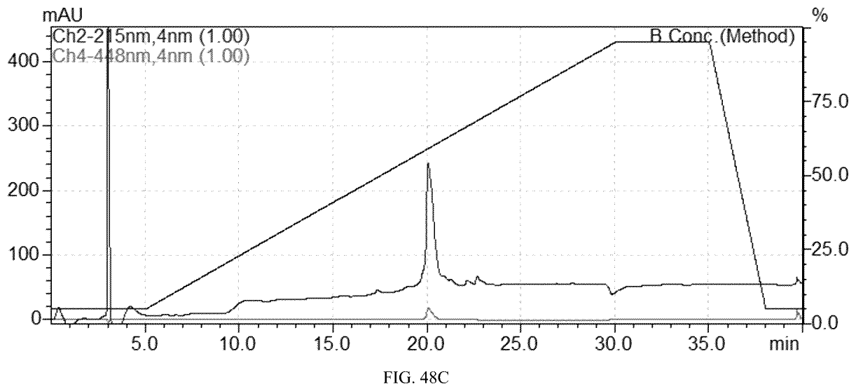

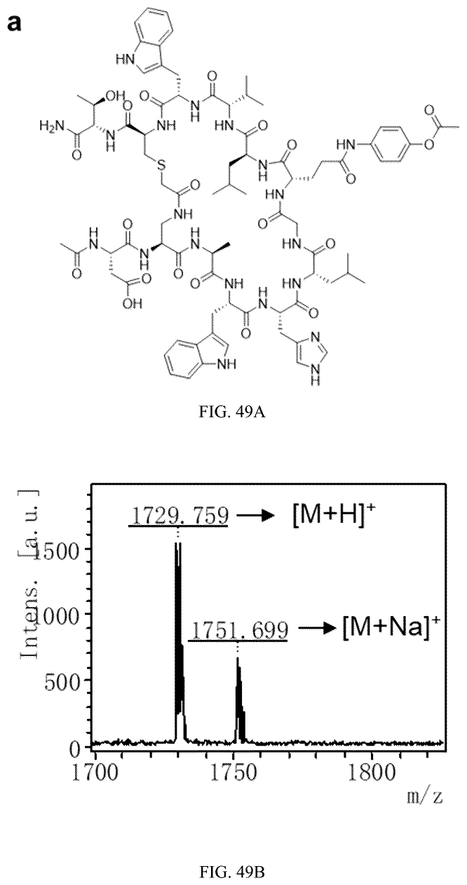

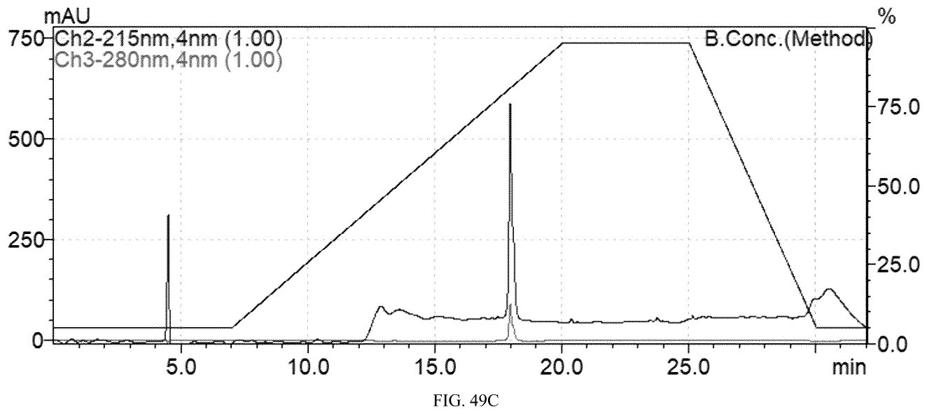

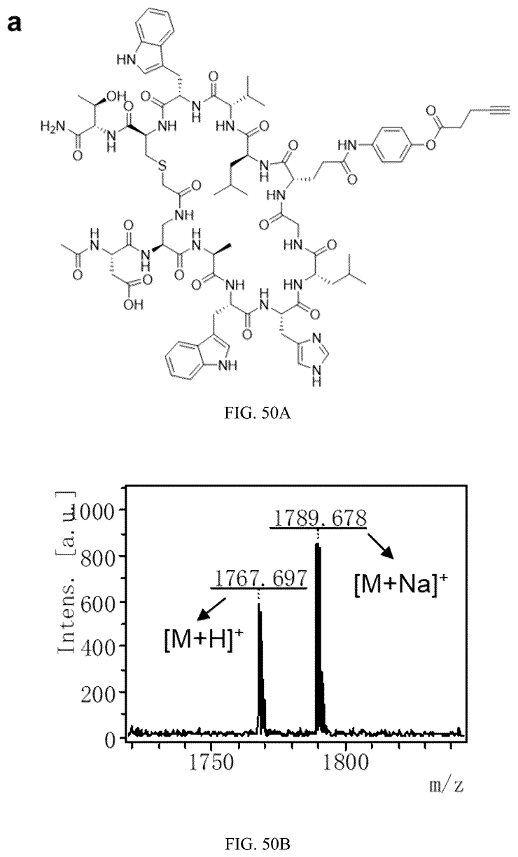

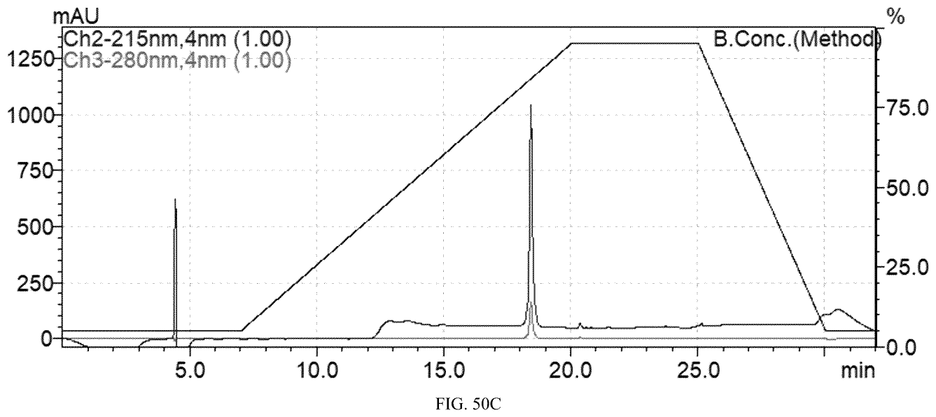

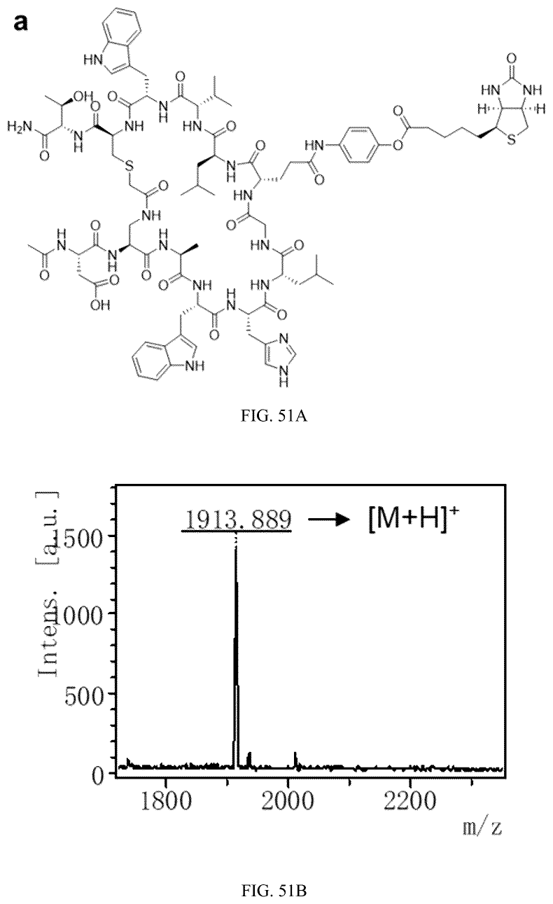

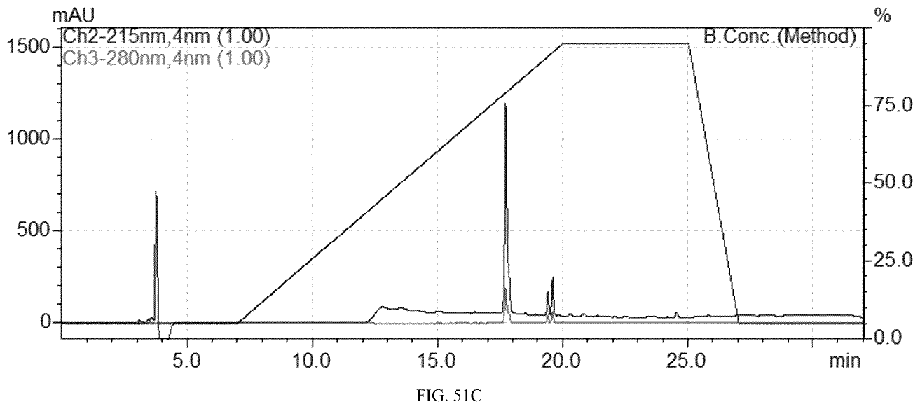

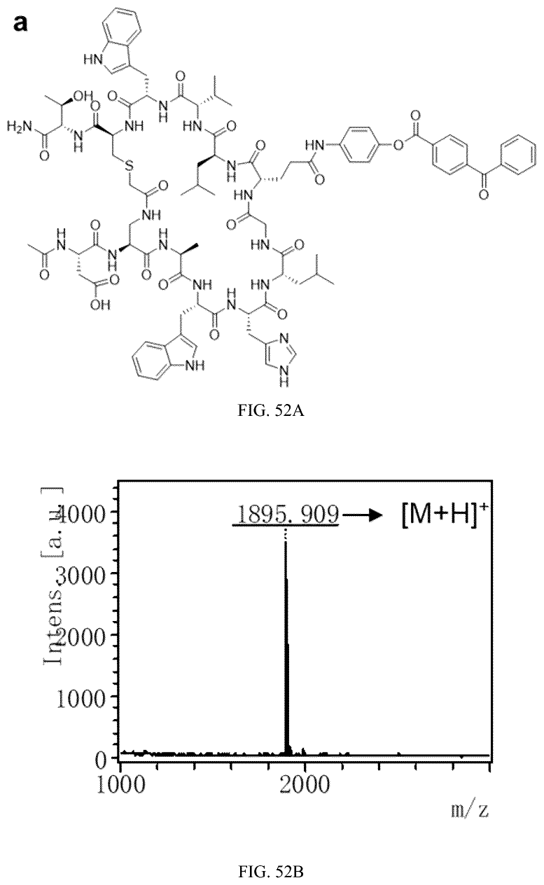

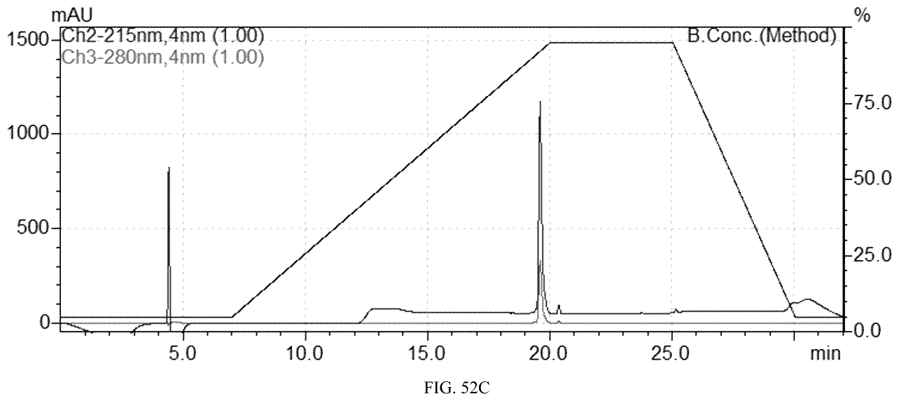

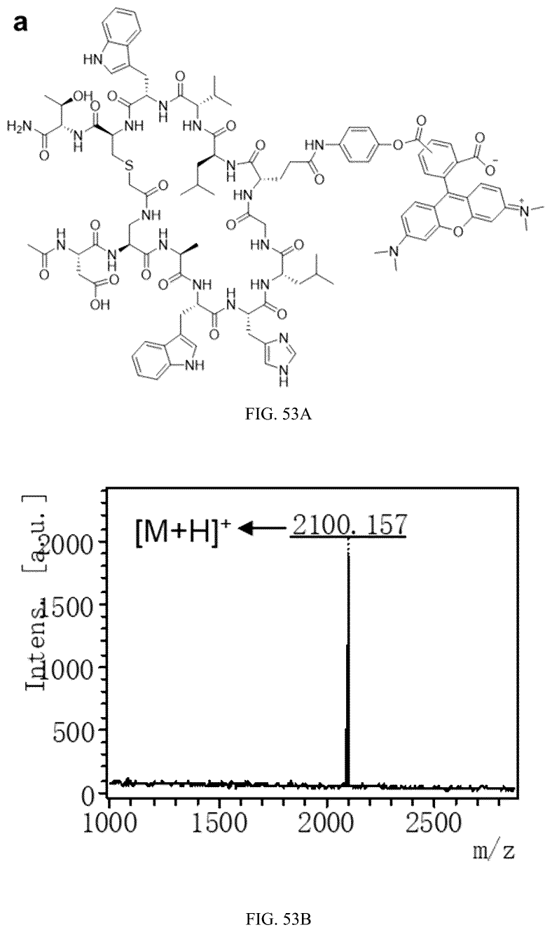

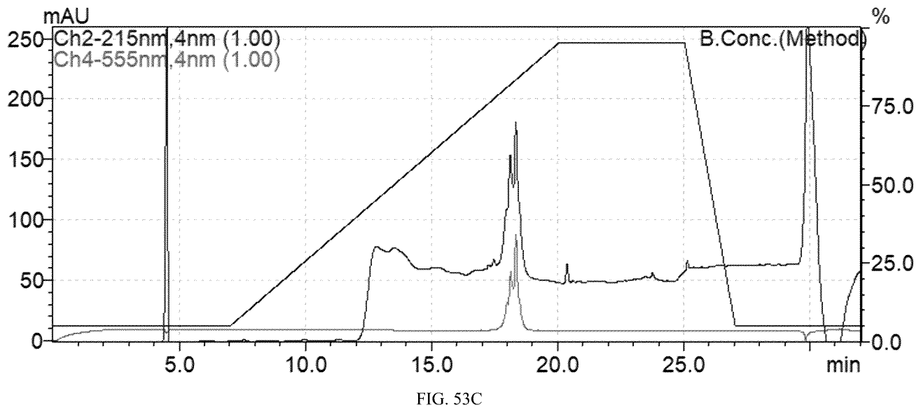

Antibody lipidation is vital to construct immunoliposomes for targeted delivery of anti-tumor drugs to the cancer cells [27]. The azidoacetylated IgG can conjugate with a lipid through SPAAC [28]. Firstly, azidoacetylated trastuzumab was incubated with DSPE-PEG2000-DBCO for 2 h. About 80% of trastuzumab can be modified by one lipid molecule according to SDS-PAGE (FIG. 6A). Fluorescently labeled liposome was then prepared by DSPE (1,2-Distearoyl-sn-glycero-3-phosphoethanolamine), cholesterol, and NBD-PE (molar ratio: 67:30:3). The immunoliposome was next prepared by incubating the trastuzumab-DSPE conjugate with the liposome for 30 min to complete the fusion (FIG. 6B) [29] (details of liposome and immunoliposome formation were shown in FIG. 41). The trastuzumab-liposome preparation was incubated with HER2-positive SK-OV-3 cells with trastuzumab-free liposomes as a control to compare their fusion efficiency. Fluorescent microscopy images showed that trastuzumab-liposomes can fuse with SK-OV-3 cells more efficiently than trastuzumab-free liposomes (FIG. 6C).

### Example 5—Synthesis of bsABCs

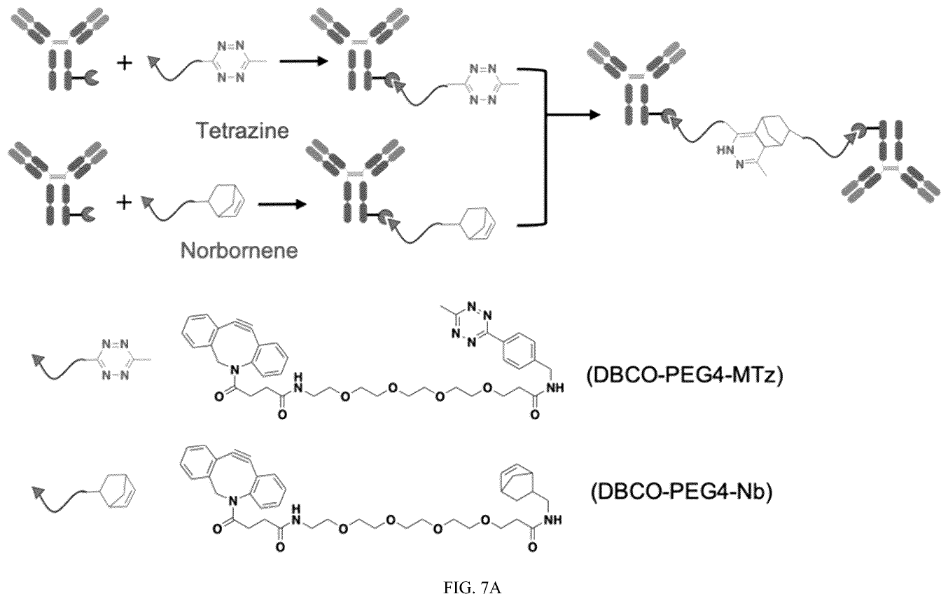

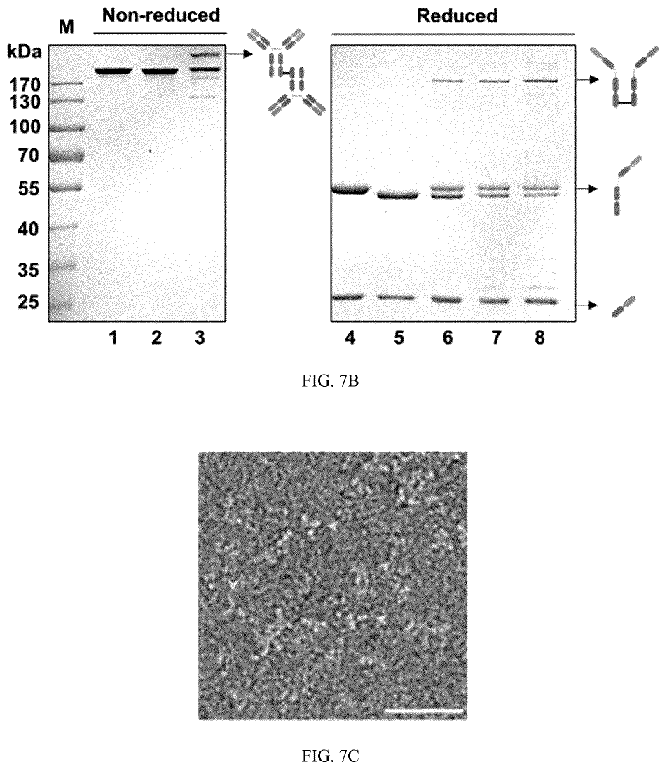

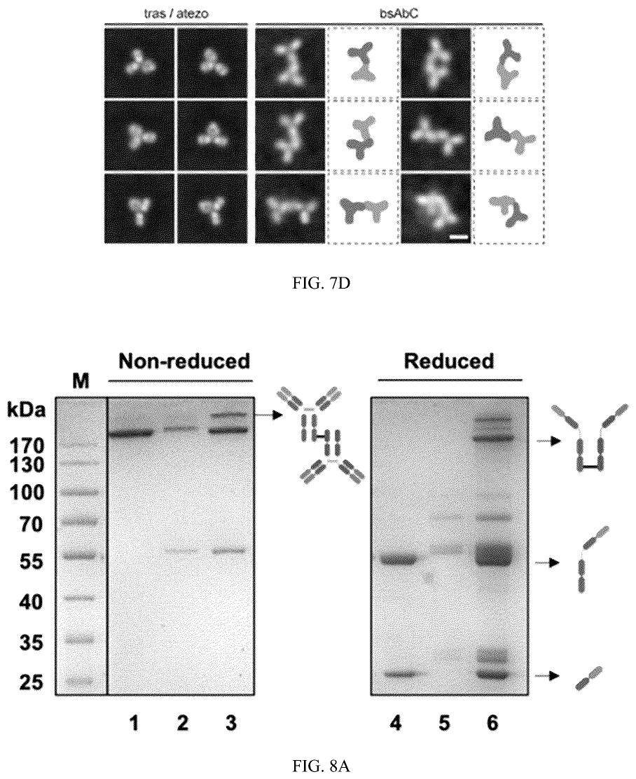

Next, we constructed bsAbCs by covalently linking two Fc domains of different IgGs, a Her2-binding trastuzumab (tras) and a PD-L1-binding atezolizumab (atezo) as a model (FIG. 7A). First, both antibodies were azidoacetylated to give respectively tras-N3 and atezo-N3. Two bifunctional linkers DBCO-PEG4-methyl tetrazine (DBCO-PEG4-MTz) and DBCO-PEG4-norborene (DBCO-PEG4-Nb) were used to functionalize tras-N3 and atezo-N3 to achieve tras-MTz and atezo-Nb. The excessive linker was removed by an Amicon 30K concentrator column (Millipore). The two antibody conjugates tras-MTz and atezo-Nb were mixed at a 1:1 ratio at 4 mg/mL and incubated at 37° C. for the inverse electron demand Diels-Alder reaction (IEDDA). Monitored by SDS-PAGE, the IEDDA reaction gave a band over 170 kDa under the non-reducing condition, corresponding to the molecular weight of tras-atezo bsAbC, at a yield of about 50% after 48 h (FIG. 7B).

### Example 6—EM OF bsABCs

The resulting bsAbC was directly visualized by negative stain electron microscopy (EM). Single particle analysis revealed the presence of both monomeric and dimeric IgGs, presumably corresponding to the tras/atezo conjugates and the bsAbCs, respectively, at a ratio of 9:1 (FIG. 7C-7D). The apparent low yield of the reaction can be explained by the heterogeneity and dynamics of the dimeric bsAbC introduced by the flexible linker, which likely led to suboptimal alignment and classification of the particles, as evident from the blurred class averages of bsAbC with fewer structural details when compared to the monomeric counterpart.

### Example 7—T Cell Activation Through bsAbCs

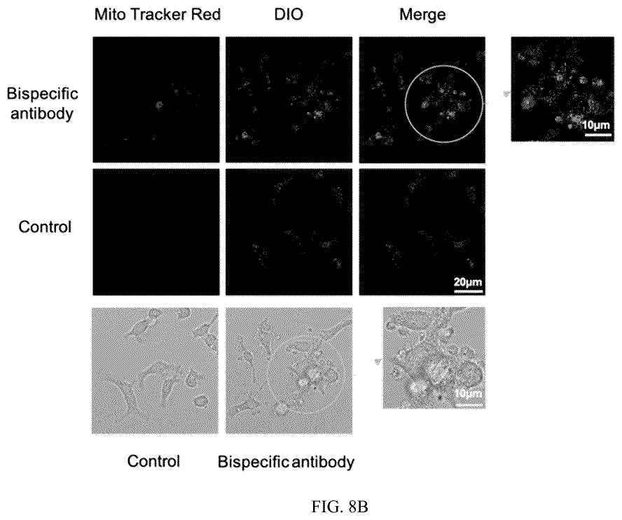

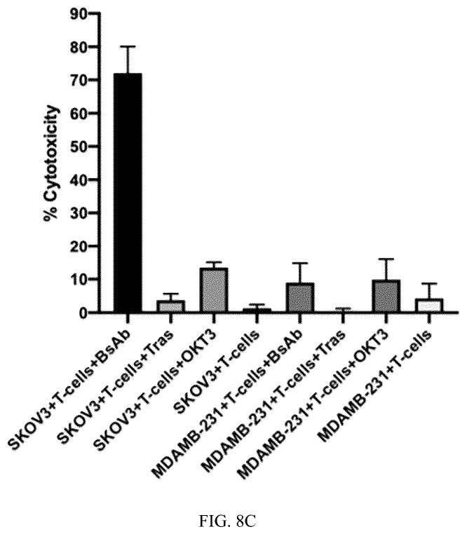

Next, we synthesized a tras-OKT3 bsAbC that binds to Her2+ cells and T lymphocytes at the same time, linking tras with the anti-CD3 antibody muromonab-CD3 (OKT3), which specifically binds human CD3 (cluster of differentiation 3) on CD8/CD3 positive cytotoxic T lymphocytes in peripheral blood mononuclear cells (PBMCs) (FIG. 8A). The tras-OKT3 bsAbC will recruit T lymphocytes to tumor cells, activate T lymphocytes, and cause subsequent lysis of the cancer cells. To examine the function of the tras-OKT3 bsAbC with the two antigens, we visualized the cross-linking of fluorescence labeled Her2+SK-OV-3 and anti-CD3+ Jurkat cells in the presence of the tras-OKT3 bsAbC. Specifically, SK-OV-3 cells and Jurkat cells were first stained with 3,3′-Dioctadecyloxacarbocyanine perchlorate (Dio) and MitoTracker Red, respectively [32]. The labeled Jurkat cells were incubated with 100 nM of the anti-HER2/anti-CD3 tras-OKT3 bsAbC in RPMI media supplemented with 10% FBS (fetal bovine serum) at 37° C. for 30 m, and the excess conjugate was washed away. A 1:1 mixture of OKT3 and tras was used as a negative control under the same labeling conditions. The cells were incubated at 37° C. for 8 h, allowing the suspension Jurkat cells to bind to the adherent SK-OV-3 cells on the plate. Unconjugated Jurkat cells were removed by gentle washing with PBS. In the presence of the tras-OKT3 bsAbC, significantly more Jurkat cells bound to the SK-OV-3 cells as compared to the co-culture incubated with the mixture of unconjugated antibodies (FIG. 8B), confirming the recruitment Jurkat cells to SK-OV-3 cells through bispecific antibody complexes.

Lastly, we demonstrated the engagement of T cells in killing target cancer cells in an in vitro effector-cell mediated cytotoxicity experiment. Human PBMCs were purified with Ficoll gradient from fresh blood of healthy donors. T cells were isolated using Human T Cell Isolation Kit (STEMCELL EasySep™). We next mixed the effector cells and the target SK-OV-3 cells at the ratio of 2 to 1 (1×10 5 to 5×10 4 cells) in RPMI media supplemented with 10% FBS in the presence of bispecific antibody mixture (25 nM). HER2-negative (MDA-MB-231) cells were used as a negative control [33]. The mixture of anti-HER2 (25 nM) and anti-CD3 (25 nM) antibodies was used as another negative control. The amount of LDH (lactate dehydrogenase) was measured as the indicator of the T cell activation. As shown in FIG. 8C, lysis of HER2+SK-OV-3 cells was observed only when the tras-OKT3 bsAbC was used. The HER2− MDA-MB-231 cells were not affected by the tras-OKT3 bsAbC. This result shows that the bsAbC can recruit the effector cells to the targeted cancer cells and result in targeted effect-cell mediated cytotoxicity, which holds promise as a therapeutic agent for cancer treatment.

Post-translational modifying enzymes achieve unparalleled site specificity that chemical conjugation cannot be on par with. On another note, attaching a reactive handle (e.g., an azide group) on IgGs at a single lysine residue enables many applications for targeted therapies. Here we converged both pursuits on a proximal acetylation reaction guided by an Fc-binding peptide and reported mono-acetylation of human IgG on Lys248 of the Fc domain, enabled by an exquisite positioning of an ester bond to the vicinity of this lysine residue. Moreover, we developed a simple and modular method for immunoliposomes and bsAbCs. Tras-OKT3 bsABC recruits cancer cells to the effector cells and induce targeted effect-cell mediated cytotoxicity. Compared to the bsAb which has gained great successes in clinical use but is very difficult to manufacture, the construction of antibody complexes based on the subject lysine acetylation reactions only requires commercially available native IgGs and can be done in a modular manner. The subject methods yield high fidelity, site-specificity acetylation.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Lu, R., Hwang, Y., Liu, I., Lee, C., Tsai, H., Li, H., & Wu, H. (2020). Development of therapeutic antibodies for the treatment of diseases. Journal Of Biomedical Science, 27(1). doi: 10.1186/s12929-019-0592-z
2. Holliger, P., & Hudson, P. (2005). Engineered antibody fragments and the rise of single domains. Nature Biotechnology, 23(9), 1126-1136. doi: 10.1038/nbt1142
3. Winter, G., & Milstein, C. (1991). Man-made antibodies. Nature, 349(6307), 293-299. doi: 10.1038/349293a0
4. Lyon, R., Setter, J., Bovee, T., Doronina, S., Hunter, J., & Anderson, M. et al. (2014). Self-hydrolyzing maleimides improve the stability and pharmacological properties of antibody-drug conjugates. Nature Biotechnology, 32(10), 1059-1062. doi: 10.1038/nbt.2968
5. Khalili, H., Godwin, A., Choi, J., Lever, R., Khaw, P., & Brocchini, S. (2013). Fab-PEG-Fab as a Potential Antibody Mimetic. Bioconjugate Chemistry, 24(11), 1870-1882. doi: 10.1021/bc400246z
6. Hull, E., Livanos, M., Miranda, E., Smith, M., Chester, K., & Baker, J. (2014). Homogeneous Bispecifics by Disulfide Bridging. Bioconjugate Chemistry, 25(8), 1395-1401. doi: 10.1021/bc5002467
7. Szijj, P., & Chudasama, V. (2021). The renaissance of chemically generated bispecific antibodies. Nature Reviews Chemistry, 5(2), 78-92. doi: 10.1038/s41570-020-00241-6
8. Badescu, G., Bryant, P., Bird, M., Henseleit, K., Swierkosz, J., & Parekh, V. et al. (2014). Bridging Disulfides for Stable and Defined Antibody Drug Conjugates. Bioconjugate Chemistry, 25(6), 1124-1136. doi: 10.1021/bc500148x
9. Bernardim, B., Matos, M., Ferhati, X., Compañón, I., Guerreiro, A., & Akkapeddi, P. et al. (2018). Efficient and irreversible antibody-cysteine bioconjugation using carbonylacrylic reagents. Nature Protocols, 14(1), 86-99. doi: 10.1038/s41596-018-0083-9
10. Kasper, M., Stengl, A., Ochtrop, P., Gerlach, M., Stoschek, T., & Schumacher, D. et al. (2019). Ethynylphosphonamidates for the Rapid and Cysteine-Selective Generation of Efficacious Antibody-Drug Conjugates. Angewandte Chemie, 131(34), 11757-11762. doi: 10.1002/ange.201904193
11. Zhou, Q., Stefano, J., Manning, C., Kyazike, J., Chen, B., & Gianolio, D. et al. (2014). Site-Specific Antibody-Drug Conjugation through Glycoengineering. Bioconjugate Chemistry, 25(3), 510-520. doi: 10.1021/bc400505q
12. Chou, C., & Lin, P. (2018). Glycan-Directed Grafting-from Polymerization of Immunoglobulin G: Site-Selectively Modified IgG-Polymer Conjugates with Preserved Biological Activity. Biomacromolecules, 19(7), 3086-3095. doi: 10.1021/acs.biomac.8b00669
13. Hui, J., Tamsen, S., Song, Y., & Tsourkas, A. (2015). LASIC: Light Activated Site-Specific Conjugation of Native IgGs. Bioconjugate Chemistry, 26(8), 1456-1460. doi: 10.1021/acs.bioconjchem.5b00275
14. Yu, C., Tang, J., Loredo, A., Chen, Y., Jung, S., & Jain, A. et al. (2018). Proximity-Induced Site-Specific Antibody Conjugation. Bioconjugate Chemistry, 29(11), 3522-3526. doi: 10.1021/acs.bioconjchem.8b00680
15. Kishimoto, S., Nakashimada, Y., Yokota, R., Hatanaka, T., Adachi, M., & Ito, Y. (2019). Site-Specific Chemical Conjugation of Antibodies by Using Affinity Peptide for the Development of Therapeutic Antibody Format. Bioconjugate Chemistry, 30(3), 698-702. doi: 10.1021/acs.bioconjchem.8b00865
16. Yamada, K., Shikida, N., Shimbo, K., Ito, Y., Khedri, Z., Matsuda, Y., & Mendelsohn, B. (2019). AJICAP: Affinity Peptide Mediated Regiodivergent Functionalization of Native Antibodies. Angewandte Chemie, 131(17), 5648-5653. doi: 10.1002/ange.201814215
17. Ohata, J., & Ball, Z. (2017). A Hexa-rhodium Metallopeptide Catalyst for Site-Specific Functionalization of Natural Antibodies. Journal Of The American Chemical Society, 139(36), 12617-12622. doi: 10.1021/jacs.7b06428
18. Shahbazian, M., & Grunstein, M. (2007). Functions of Site-Specific Histone Acetylation and Deacetylation. Annual Review Of Biochemistry, 76(1), 75-100. doi: 10.1146/annurev.biochem.76.052705.162114
19. Wang, J., Yu, Y., & Xia, J. (2013). Short Peptide Tag for Covalent Protein Labeling Based on Coiled Coils. Bioconjugate Chemistry, 25(1), 178-187. doi: 10.1021/bc400498p
20. Wang, R., Leung, P., Huang, F., Tang, Q., Kaneko, T., & Huang, M. et al. (2018). Reverse Binding Mode of Phosphotyrosine Peptides with SH2 Protein. Biochemistry, 57(35), 5257-5269. doi: 10.1021/acs.biochem.8b00677
21. Zhang, Y., Liang, Y., Huang, F., Zhang, Y., Li, X., & Xia, J. (2019). Site-Selective Lysine Reactions Guided by Protein-Peptide Interaction. Biochemistry, 58(7), 1010-1018. doi: 10.1021/acs.biochem.8b01223
22. Yu, Y., Liu, M., Ng, T., Huang, F., Nie, Y., & Wang, R. et al. (2015). PDZ-Reactive Peptide Activates Ephrin-B Reverse Signaling and Inhibits Neuronal Chemotaxis. ACS Chemical Biology, 11(1), 149-158. doi: 10.1021/acschembio.5b00889
23. Qiu, J., Nie, Y., Zhao, Y., Zhang, Y., Li, L., & Wang, R. et al. (2020). Safeguarding intestine cells against enteropathogenic Escherichia coli by intracellular protein reaction, a preventive antibacterial mechanism. Proceedings Of The National Academy Of Sciences, 117(10), 5260-5268. doi: 10.1073/pnas.1914567117
24. So, W., Zhang, Y., Kang, W., Wong, C., Sun, H., & Xia, J. (2017). Site-selective covalent reactions on proteinogenic amino acids. Current Opinion In Biotechnology, 48, 220-227. doi: 10.1016/j.copbio.2017.06.003
25. So, W., Wong, C., & Xia, J. (2018). Peptide photocaging: A brief account of the chemistry and biological applications. Chinese Chemical Letters, 29(7), 1058-1062. doi: 10.1016/j.cclet.2018.05.015
26. Huang, W., Wu, X., Gao, X., Yu, Y., Lei, H., & Zhu, Z. et al. (2019). Maleimide-thiol adducts stabilized through stretching. Nature Chemistry, 11(4), 310-319. doi: 10.1038/s41557-018-0209-2
27. Berndsen, C., & Denu, J. (2008). Catalysis and substrate selection by histone/protein lysine acetyltransferases. Current Opinion In Structural Biology, 18(6), 682-689. doi: 10.1016/j.sbi.2008.11.004
28. Shvedunova, M., & Akhtar, A. (2022). Modulation of cellular processes by histone and non-histone protein acetylation. Nature Reviews Molecular Cell Biology, 23(5), 329-349. doi: 10.1038/s41580-021-00441-y
29. McCombs, J., & Owen, S. (2015). Antibody Drug Conjugates: Design and Selection of Linker, Payload and Conjugation Chemistry. The AAPS Journal, 17(2), 339-351. doi: 10.1208/s12248-014-9710-8
30. DeLano, W., Ultsch, M., de, A., Vos, & Wells, J. (2000). Convergent Solutions to Binding at a Protein-Protein Interface. Science, 287(5456), 1279-1283. doi: 10.1126/science.287.5456.1279
31. Hughes, C., Yang, Y., Liu, W., Dorrestein, P., Clair, J., & Fenical, W. (2009). Marinopyrrole A Target Elucidation by Acyl Dye Transfer. Journal Of The American Chemical Society, 131(34), 12094-12096. doi: 10.1021/ja903149u
32. Takaoka, Y., Nishikawa, Y., Hashimoto, Y., Sasaki, K., & Hamachi, I. (2015). Ligand-directed dibromophenyl benzoate chemistry for rapid and selective acylation of intracellular natural proteins. Chemical Science, 6(5), 3217-3224. doi: 10.1039/c5sc00190k
33. Martos-Maldonado, M., Hjuler, C., Ssrensen, K., Thygesen, M., Rasmussen, J., & Villadsen, K. et al. (2018). Selective N-terminal acylation of peptides and proteins with a Gly-His tag sequence. Nature Communications, 9(1). doi: 10.1038/s41467-018-05695-3
34. Vidarsson, G., Dekkers, G., & Rispens, T. (2014). IgG Subclasses and Allotypes: From Structure to Effector Functions. Frontiers In Immunology, 5. doi: 10.3389/fimmu.2014.00520
35. Jung, Y., Kang, H., Lee, J., Jung, S., Yun, W., Chung, S., & Chung, B. (2008). Controlled antibody immobilization onto immunoanalytical platforms by synthetic peptide. Analytical Biochemistry, 374(1), 99-105. doi: 10.1016/j.ab.2007.10.022
36. Herbst, R., Soria, J., Kowanetz, M., Fine, G., Hamid, O., & Gordon, M. et al. (2014).

Predictive correlates of response to the anti-PD-L1 antibody MPDL3280A in cancer patients. Nature, 515(7528), 563-567. doi: 10.1038/nature14011
37. Santini, F., & Rudin, C. (2017). Atezolizumab for the treatment of non-small cell lung cancer. Expert Review Of Clinical Pharmacology, 10(9), 935-945. doi: 10.1080/17512433.2017.1356717
38. Allen, T., & Cullis, P. (2013). Liposomal drug delivery systems: From concept to clinical applications. Advanced Drug Delivery Reviews, 65(1), 36-48. doi: 10.1016/j.addr.2012.09.037
39. Di, J., Xie, F., & Xu, Y. (2020). When liposomes met antibodies: Drug delivery and beyond. Advanced Drug Delivery Reviews, 154-155, 151-162. doi: 10.1016/j.addr.2020.09.003
40. Nellis, D., Giardina, S., Janini, G., Shenoy, S., Marks, J., & Tsai, R. et al. (2008). Preclinical Manufacture of Anti-HER2 Liposome-Inserting, scFv-PEG-Lipid Conjugate. 2. Conjugate Micelle Identity, Purity, Stability, and Potency Analysis. Biotechnology Progress, 21(1), 221-232. doi: 10.1021/bp049839z
41. Oliveira, B., Guo, Z., & Bernardes, G. (2017). Inverse electron demand Diels-Alder reactions in chemical biology. Chemical Society Reviews, 46(16), 4895-4950. doi: 10.1039/c7cs00184c
42. Patterson, D., Nazarova, L., & Prescher, J. (2014). Finding the Right (Bioorthogonal) Chemistry. ACS Chemical Biology, 9(3), 592-605. doi: 10.1021/cb400828a
43. Kim, C., Axup, J., Dubrovska, A., Kazane, S., Hutchins, B., & Wold, E. et al. (2012). Synthesis of Bispecific Antibodies using Genetically Encoded Unnatural Amino Acids. Journal Of The American Chemical Society, 134(24), 9918-9921. doi: 10.1021/ja303904e
44. Warwas, K., Meyer, M., Gongalves, M., Moldenhauer, G., Bulbuc, N., & Knabe, S. et al. (2021). Co-Stimulatory Bispecific Antibodies Induce Enhanced T Cell Activation and Tumor Cell Killing in Breast Cancer Models. Frontiers In Immunology, 12. doi: 10.3389/fimmu.2021.719116
45. Zivanov, J. et al. New tools for automated high-resolution cryo-EM structure determination in relion-3. eLife 7, (2018).
46. Punjani, A., et al. cryoSPARC: algorithms for rapid unsupervised cryo-EM structure determination. Nat. Methods 14, (2017).

We claim:

1. A modified Fc-III peptide according to SEQ ID NO: 4, wherein residue His5, Lys6, or Glu8 is substituted with a glutamine derivative containing a phenyl azidoacetate motif at a side chain.

2. The modified Fc-III peptide of claim 1, wherein the Fc-III peptide is F1, according to formula (I); F2, according to formula (II); F3, according to formula (III); F6, according to formula (IV); f-F0, according to formula (V); f-F4, according to formula (VI); f-F5, according to formula (VII); F7, according to formula (VIII); F8, according to formula (IX); F9, according to formula (X); F10, according to formula (XI); or F11, according to formula (XII):

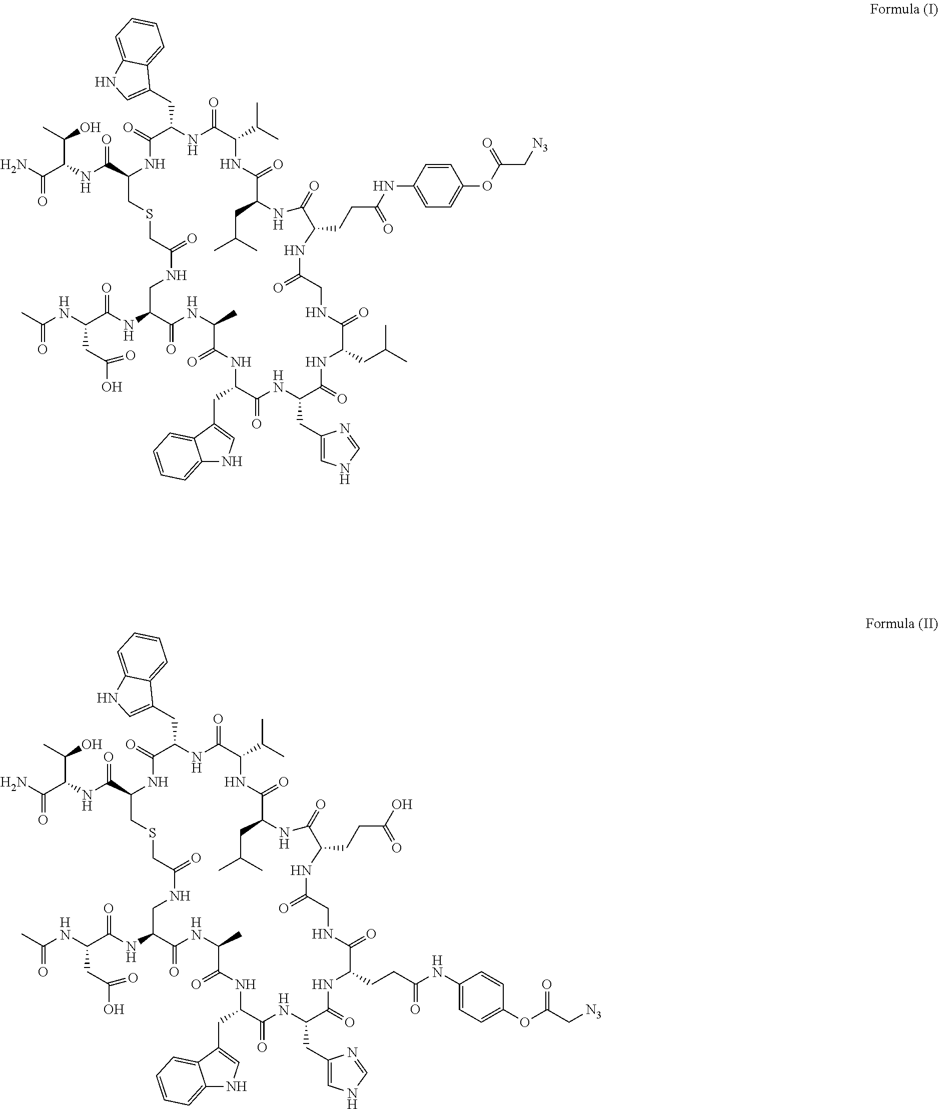

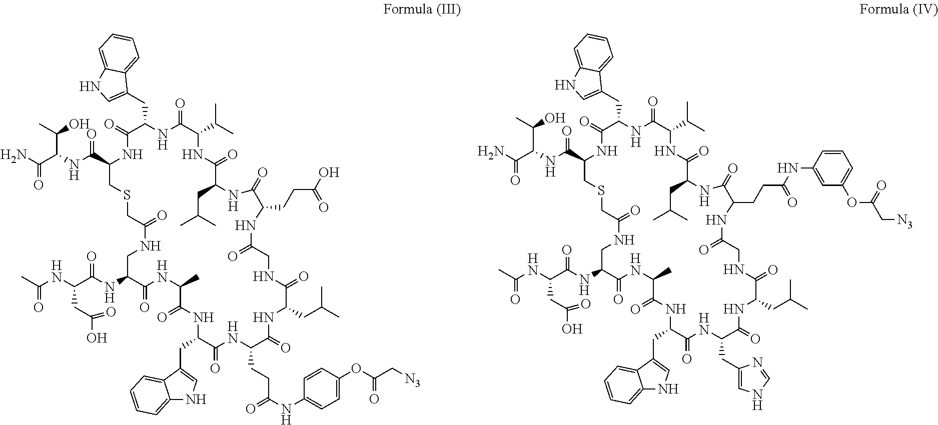

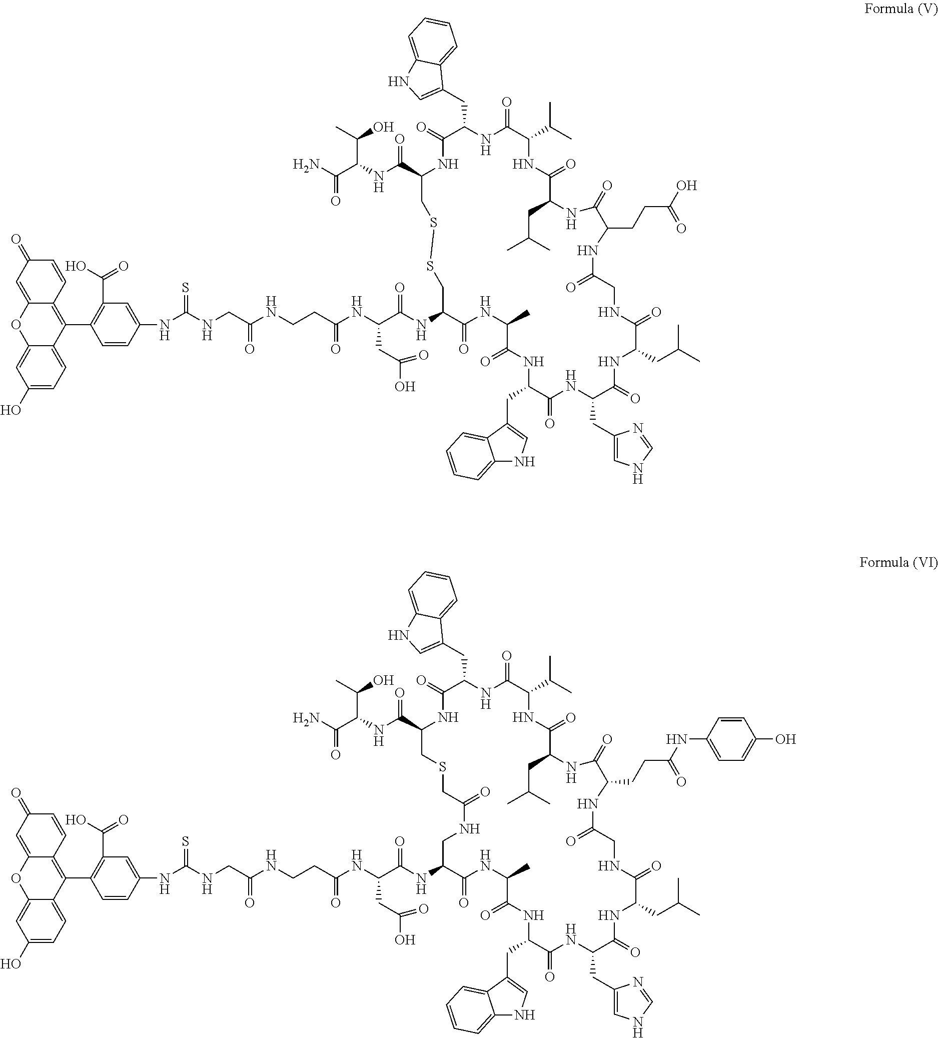

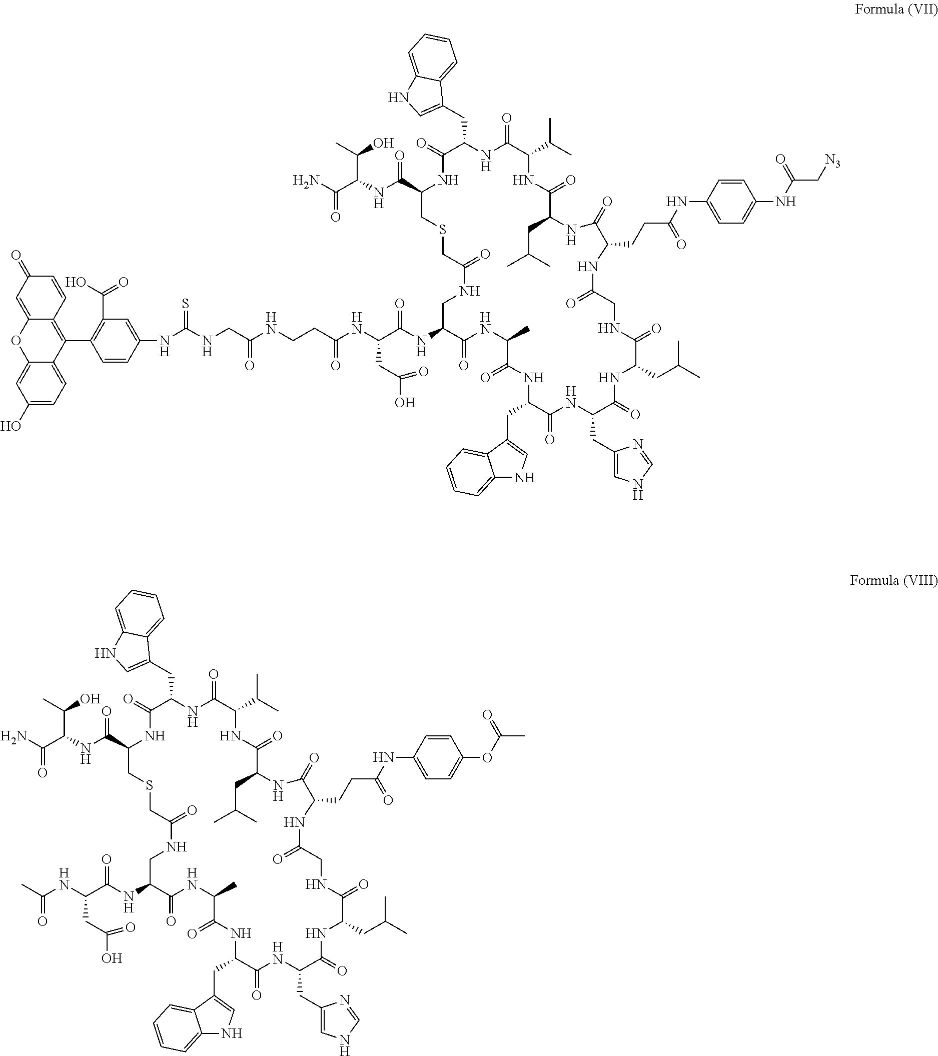

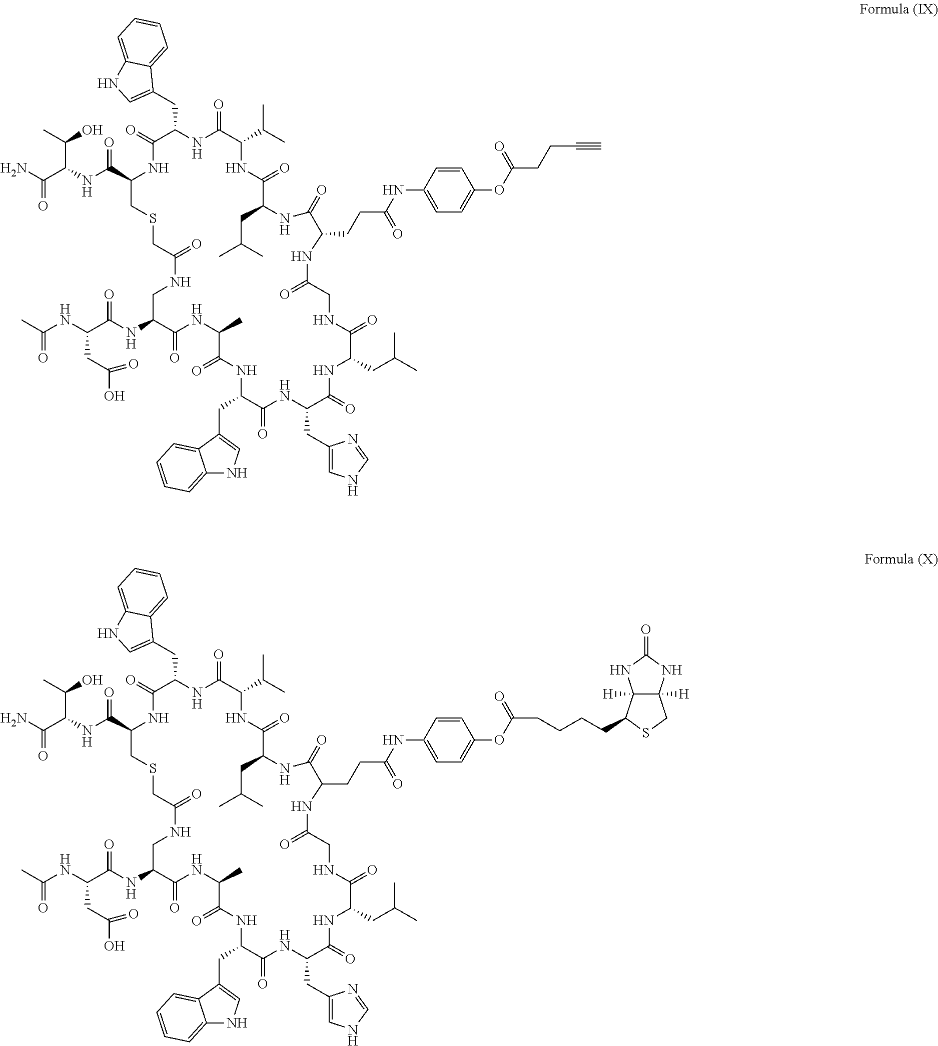

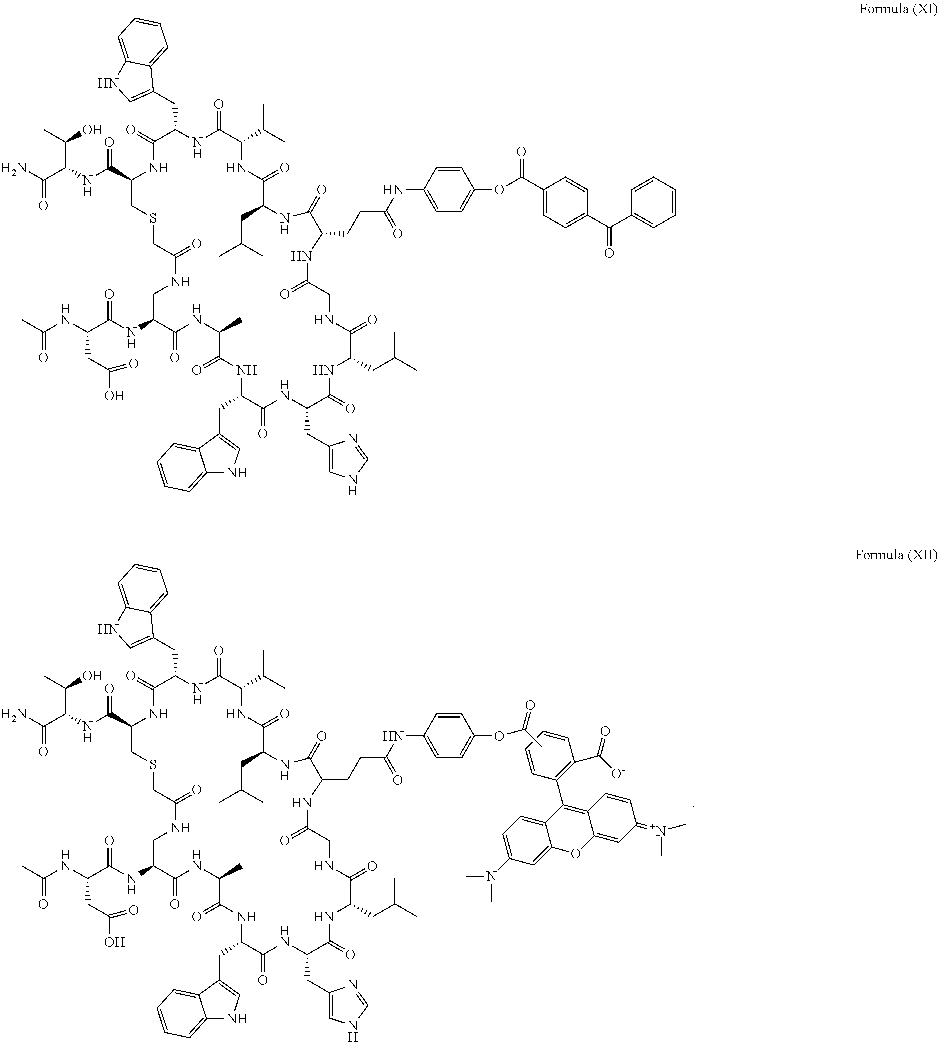

3. A method of synthesizing an antibody-lipid conjugate, comprising:
a) incubating a modified Fc-III peptide according to SEQ ID NO: 4, wherein residue His5, Lys6, or Glu8 is substituted with a glutamine derivative containing a phenyl azidoacetate motif at a side chain with an antibody, yielding an acetylated antibody;
b) incubating the acetylated antibody with a functionalized lipid to yield the antibody-lipid conjugate; and
c) optionally, incubating the antibody-lipid conjugate with a liposome to yield an antibody-liposome conjugate.

4. The method of claim 3, wherein the incubation of steps a), b), or c) occurs in a buffer solution at about 30° C. to about 40° C. for about 1 min to about 6 h.

5. The method of claim 4, wherein the buffer is PBS.

6. The method of claim 3, wherein the acetylated antibody is an azidoacetylated antibody.

7. The method of claim 3, wherein the functionalized lipid is DSPE-PEG2000-DBCO.

8. The method of claim 3, wherein the liposome comprises 1-palmitoyl-2-oleoyl-glycero-3-phosphocholine, cholesterol, and L-α-Phosphatidylethanolamine-N-(7-nitro-2-1,3-benzoxadiazol-4-yl) in a molar ratio of 67:30:3, respectively.

9. The method of claim 3, wherein the modified Fc-III peptide is F1, according to formula

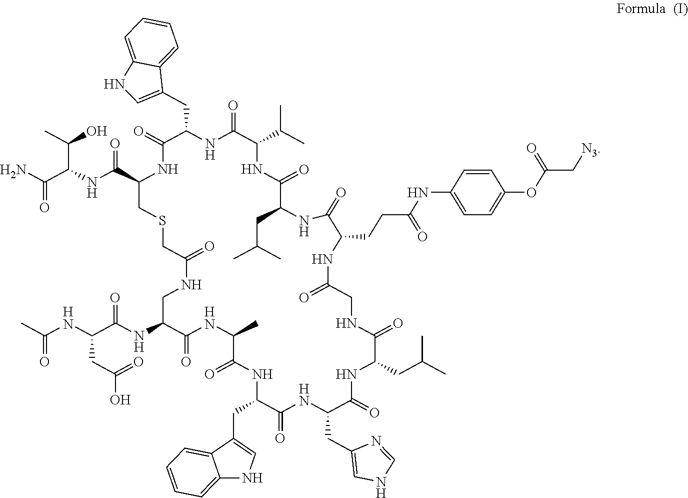 [SMILES: CC(=O)N[C@@H](CC(=O)O)C(=O)N[C@H]1CNC(=O)CSC[C@@H](C(=O)N[C@H](C(N)=O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)Nc2ccc(OC(=O)CN=[N+]=[N-])cc2)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O]

10. A method of synthesizing a bispecific antibody complex (bsAbC), comprising:
a) incubating a modified Fc-III peptide according to SEQ ID NO: 4, wherein residue His5, Lys6, or Glu8 is substituted with a glutamine derivative containing a phenyl azidoacetate motif at a side chain with a first antibody, yielding a first acetylated antibody;
b) incubating the modified Fc-III peptide according to SEQ ID NO: 4, wherein residue His5, Lys6, or Glu8 is substituted with a glutamine derivative containing a phenyl azidoacetate motif at a side chain with a second antibody, yielding a second acetylated antibody;
c) incubating the first acetylated antibody with a first bifunctional linker, yielding a first antibody conjugate;
d) incubating the second acetylated antibody with a second bifunctional linker, yielding a second antibody conjugate; and
e) mixing the first antibody conjugate and the second antibody conjugate, yielding a bsAbC.

11. The method of claim 10, wherein steps a), b), c), d), or any combination thereof occur in a buffer solution at about 30° C. to about 40° C. for about 1 min to about 6 h.

12. The method of claim 11, wherein the buffer solution is PBS.

13. The method of claim 10, wherein the acetylated antibody is an azidoacetylated antibody.

14. The method of claim 10, wherein the first bifunctional linker is distinct from the second bifunctional linker and the first bifunction linker or the second bifunctional linker is DBCO-PEG4-methyl tetrazine (DBCO-PEG4-MTz) or DBCO-PEG4-norborene (DBCO-PEG4-Nb).

15. The method of claim 10, wherein the first antibody conjugate and the second antibody conjugate are mixed at a ratio of at a 1:1 ratio and incubated at 30° C. to about 40° C. for about 1 min to about 120 h.

16. The method of claim 10, wherein the modified Fc-III peptide is F1, according to formula (I):

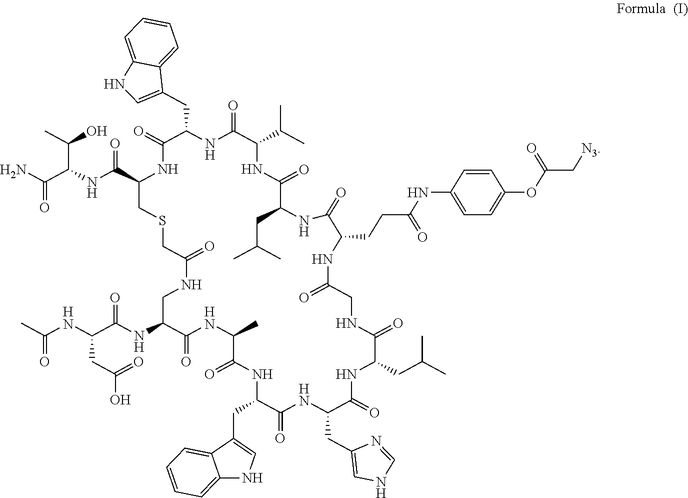 [SMILES: CC(=O)N[C@@H](CC(=O)O)C(=O)N[C@H]1CNC(=O)CSC[C@@H](C(=O)N[C@H](C(N)=O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)Nc2ccc(OC(=O)CN=[N+]=[N-])cc2)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O]

17. A method of treating cancer, comprising:
administering a composition comprising the antibody-lipid conjugate synthesized according to claim 3.

18. The method of claim 17, wherein the antibody targets an oncogene.

19. The method of claim 18, wherein the oncogene is HER2.

20. A method of treating cancer, comprising:
administering a composition comprising the bsAbC synthesized according to claim 10.

21. The method of claim 20, wherein the first antibody is an anti-CD3 or an anti-PDL1 antibody and the second antibody targets an oncogene.

22. The method of claim 21, wherein the oncogene is HER2.
